(19) United States
(12) Patent Application Publication
(10) Pub. No.: US 2007/0278461 A1
(43) Pub. Date: Dec. 6, 2007

(54) PHOTOCHROMIC MATERIALS COMPRISING HALOALKYL GROUPS

(75) Inventors: Olga G. Petrovskaia, Acton, MA (US); Anu Chopra, Pittsburgh, PA (US); Patrick M. Brown, Moon Township, PA (US)

Correspondence Address: Deborah M. Altman;PPG Industries, Inc., Law Department - Intellectual Property, One PPG Place - 39th Floor, Pittsburgh, PA, 15272-0001, US

(73) Assignee: TRANSITIONS OPTICAL, INC., Pinellas Park, FL (US)

(21) Appl. No.: 11/695,126

(22) Filed: Apr. 2, 2007

Related U.S. Application Data
(60) Provisional application No. 60/809,732, filed on May 31, 2006.

(51) Int. Cl. C08L 73/00; C07D 309/00; C07D 413/02

(52) U.S. Cl. 252/586; 544/149; 549/214; 549/356; 549/428

(57) ABSTRACT

Various non-limiting embodiments of the present invention relate to photochromic materials which include a haloalkyl group. More particularly, various non-limiting embodiments disclosed herein provide photochromic materials including an indeno-fused naphthopyran, such as, an indeno[2′,3′:3,4]naphtho[1,2-b] pyran, and a haloalkyl group bonded at the 13-position thereof, wherein the haloalkyl group is a perhalogenated group or a group represented by —O(CH₂)ₐ(CX₂)_{b}CT₃, wherein T is a halogen, each X is independently hydrogen or halogen, a is an integer ranging from 1 to 10, and b is an integer ranging from 1 to 10. Other non-limiting embodiments disclosed herein provide photochromic composition and photochromic articles, such as, but not limited to ophthalmic lens, which include the disclosed photochromic materials and methods of making the same.

### CROSS REFERENCE TO RELATED APPLICATION

This is a non-provisional application claiming priority under 35 U.S.C. §119(e)(1) of U.S. Provisional Patent Application Ser. No. 60/809,732 filed May 31, 2006.

### BACKGROUND

The present invention generally relates to photochromic materials, and more particularly relates to photochromic materials comprising an indeno[2′,3′:3,4]naphtho[1,2-b]pyran comprising a haloalkyl group bonded at the 13-position thereof. The present invention further relates to photochromic compositions and articles that comprise such photochromic materials.

Photochromic materials undergo a transformation from one form (or state) to another in response to certain wavelengths of electromagnetic radiation, with each form having a characteristic absorption spectrum for visible radiation. For example, thermally reversible photochromic materials are capable of transforming from a ground-state form to an activated-state form in response to actinic radiation, and reverting back to the ground-state form in response to thermal energy and in the absence of the actinic radiation. As used herein, the term “actinic radiation” refers to electromagnetic radiation that is capable of causing a photochromic material to transform from one form or state to another.

Photochromic materials adapted for use in ophthalmic applications appear to be essentially colorless or “optically clear” when not exposed to actinic radiation (i.e., in the ground-state form) and exhibit a visible color that is characteristic of the absorption spectrum of the activated-state form of the photochromic material upon exposure to actinic radiation. Photochromic compositions and articles that contain one or more photochromic materials, for example, photochromic lenses for eyewear applications, may display clear and colored states that generally correspond to the optically clear and colored states of the photochromic material(s) that they contain.

For certain applications, it is desirable that the photochromic material be able to make the transition from the optically clear, ground-state form to the colored, activated-state form as quickly as possible. Additionally, for certain applications it is desirable that the photochromic material be able to make the transition from the colored, activated-state form to the optically clear, ground-state form as quickly as possible. For example, in photochromic eyewear applications, ophthalmic lenses comprising photochromic materials may transform from a clear state to a colored state as the wearer moves from a region of low actinic radiation, such as, indoors, to a region of high actinic radiation, such as, in direct sunlight. As the lenses become colored, less electromagnetic radiation having wavelengths within the visible and/or ultraviolet regions of the electromagnetic spectrum is transmitted through the lens to the wearer's eyes. In other words, more electromagnetic radiation is absorbed by the lenses in the colored state than in the clear state. When the wearer subsequently moves from the region of high actinic radiation back to a region of low actinic radiation, the photochromic material in the eyewear may revert from the colored, activated-state form to the optically clear, ground-state form in the absence of actinic radiation and absorbance of thermal energy. If the transition from the optically clear state to the colored state takes several minutes or more upon exposure to actinic radiation, the benefit of the reduced transmittance of visible and/or ultraviolet radiation that may be derived from the lenses in the colored state may be diminished. Further, if the transition from the colored state to the optically clear state takes several minutes or more once removed from actinic radiation, the wearer's vision may be less than optimal during this time due to the combined effects of the lower ambient light and the reduced transmission of visible light through the colored lenses. Accordingly, for certain applications, it would be desirable to develop photochromic materials that may more quickly transition from the optically clear, ground-state form to the colored, activated-state form, as well as from the colored, activated-state form to the optically clear, ground-state form.

Further, although conventional photochromic materials adapted for use in ophthalmic applications appear essentially colorless or optically clear when not exposed to actinic radiation, most conventional photochromic materials absorb at least some amount of visible radiation in their ground-state form. For certain applications, however, it may be desirable to limit the amount of visible radiation absorbed by the photochromic material in the ground-state form to provide as high a transmittance of visible light as possible. For example, in ophthalmic lens applications, it is generally desirable that a lens substrate comprising the photochromic material have a transmittance for visible radiation when the photochromic material is in the optically clear, ground-state form that is substantially equivalent to the lens substrate without the photochromic material. That is, for ophthalmic applications it is generally desirable that the presence of the photochromic material in or on the lens does not negatively impact the visual acuity and visual comfort provided by the lens when the photochromic material is in the optically clear, ground-state form.

Accordingly, there is a need for photochromic materials that may exhibit a high transmittance of visible radiation in their optically clear, ground-state form; may transition quickly from their optically clear, ground-state form to their colored, activated-state form; and may transition quickly from their colored, activated-state form to their optically clear, ground-state form.

### BRIEF SUMMARY OF THE DISCLOSURE

Various non-limiting embodiments of the present invention relate to photochromic materials comprising a haloalkyl group. For example, various non-limiting embodiments disclosed herein provide a photochromic material comprising: an indeno[2′,3′:3,4]naphtho[1,2-b]pyran and a haloalkyl group bonded at the 13-position of the indeno[2′,3′:3,4]naphtho[1,2-b]pyran. According to certain non-limiting embodiments, the haloalkyl group is: a perhalogenated group that is at least one of a perhalo(C₁-C₁₀)alkyl, a perhalo(C₂-C₁₀)alkenyl, a perhalo(C₃-C₁₀)alkynyl, a perhalo(C₁-C₁₀)alkoxy or a perhalo(C₃-C₁₀)cycloalkyl; or a group represented by —O(CH₂)ₐ(CX₂)_{b}CT₃, wherein T is a halogen, each X is independently hydrogen or halogen, a is an integer ranging from 1 to 10, and b is an integer ranging from 1 to 10.

Further non-limiting embodiments disclosed herein provide a photochromic indeno[2′,3′:3,4]naphtho[1,2-b]pyran comprising a haloalkyl group bonded at the 13-position of the indeno[2′,3′:3,4]naphtho[1,2-b]pyran, wherein the haloalkyl group is: a perhalogenated group that is at least one of a perhalo(C₁-C₁₀)alkyl, a perhalo(C₂-C₁₀)alkenyl, a perhalo(C₃-C₁₀)alkynyl, a perhalo(C₁-C₁₀)alkoxy or a perhalo(C₃-C₁₀)cycloalkyl; or a group represented by —O(CH₂)ₐ(CX₂)_{b}CT₃, wherein T is a halogen, each X is independently hydrogen or halogen, a is an integer ranging from 1 to 10, and b is an integer ranging from 1 to 10.

Still further non-limiting embodiments provide for a photochromic material represented by structure (iv):

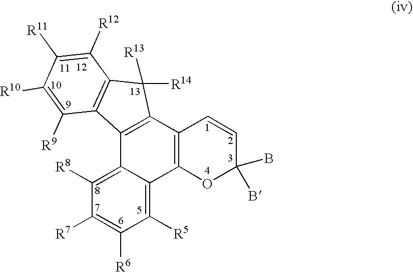

wherein:

R¹³ and R¹⁴ are each independently: a perhalogenated group that is at least one of a perhalo(C₁-C₁₀)alkyl, a perhalo(C₂-C₁₀)alkenyl, a perhalo(C₃-C₁₀)alkynyl, a perhalo(C₁-C₁₀)alkoxy or a perhalo(C₃-C₁₀)cycloalkyl; a group represented by —O(CH₂)ₐ(CX₂)_{b}CT₃, wherein T is a halogen, each X is independently hydrogen or halogen, a is an integer ranging from 1 to 10, and b is an integer ranging from 1 to 10; a silicon-containing group represented by one of

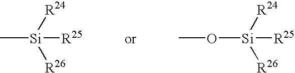

wherein R²⁴, R²⁵, and R²⁶ are each independently C₁-C₁₀ alkyl, C₁-C₁₀ alkoxy or phenyl; a metallocenyl group; a reactive substituent or a compatiblizing substituent; hydrogen, hydroxy, C₁-C₆ alkyl, chloro, fluoro, C₃-C₇ cycloalkyl, allyl or C₁-C₈ haloalkyl; morpholino, piperidino, pyrrolidino, an unsubstituted, mono- or di-substituted amino, wherein said amino substituents are each independently C₁-C₆ alkyl, phenyl, benzyl or naphthyl; an unsubstituted, mono-, di- or tri-substituted aryl group chosen from phenyl, naphthyl, benzyl, phenanthryl, pyrenyl, quinoyl, isoquinolyl, benzofuranyl, thienyl, benzothienyl, dibenzofuranyl, dibenzothienyl, carbazolyl or indolyl, wherein the aryl group substituents are each independently halogen, C₁-C₆ alkyl or C₁-C₆ alkoxy; —C(═O)R²⁷, wherein R²⁷ is hydrogen, hydroxy, C₁-C₆ alkyl, C₁-C₆ alkoxy, amino, mono- or di-(C₁-C₆)alkylamino, morpholino, piperidino, pyrrolidino, an unsubstituted, mono- or di-substituted phenyl or naphthyl, an unsubstituted, mono- or di-substituted phenoxy or an unsubstituted, mono- or di-substituted phenylamino, wherein said phenyl, naphthyl, phenoxy and phenylamino substituents are each independently C₁-C₆ alkyl or C₁-C₆ alkoxy; —OR²⁸, wherein R²⁸ is C₁-C₆ alkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl, C₁-C₆ alkoxy(C₂-C₄)alkyl, C₃-C₇ cycloalkyl, mono(C₁-C₄)alkyl substituted C₃-C₇ cycloalkyl, C₁-C₈ chloroalkyl, C₁-C₈ fluoroalkyl, allyl or C₁-C₆ acyl, —CH(R²⁹)R³⁰, wherein R²⁹ is hydrogen or C₁-C₃ alkyl, R³⁰ is —CN, —CF₃ or —COOR³¹, wherein R³¹ is hydrogen or C₁-C₃ alkyl, or —C(═O)R³², wherein R³² is hydrogen, C₁-C₆ alkyl, C₁-C₆ alkoxy, amino, mono- or di-(C₁-C₆)alkylamino, an unsubstituted, mono- or di-substituted phenyl or naphthyl, an unsubstituted, mono- or di-substituted phenoxy or an unsubstituted, mono- or di-substituted phenylamino, wherein said phenyl, naphthyl, phenoxy and phenylamino substituents are each independently C₁-C₆ alkyl or C₁-C₆ alkoxy; a 4-substituted phenyl, the substituent being a dicarboxylic acid residue or derivative thereof, a diamine residue or derivative thereof, an amino alcohol residue or derivative thereof, a polyol residue or derivative thereof, —(CH₂)—, —(CH₂)ₑ— or —┌O—(CH₂)ₑ┐_{f}—, wherein e is an integer ranging from 2 to 6 and f is an integer ranging from 1 to 50, and wherein the substituent is connected to an aryl group of another photochromic material; —CH(R³³)₂, wherein R³³ is —CN or —COOR³⁴, wherein R³⁴ is hydrogen, C₁-C₆ alkyl, C₃-C₇ cycloalkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl or an unsubstituted, mono- or di-substituted phenyl or naphthyl, wherein said phenyl and naphthyl substituents are each independently C₁-C₆ alkyl or C₁-C₆ alkoxy; or —CH(R³⁵)R³⁶, wherein R³⁵ is hydrogen, C₁-C₆ alkyl or an unsubstituted, mono- or di-substituted phenyl or naphthyl, wherein said phenyl and naphthyl substituents are each independently C₁-C₆ alkyl or C₁-C₆ alkoxy, and R³⁶ is —C(═O)OR³⁷, —C(═O)R³⁸ or —CH₂OR³⁹ wherein: R³⁷ is hydrogen, C₁-C₆ alkyl, C₃-C₇ cycloalkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl or an unsubstituted, mono- or di-substituted phenyl or naphthyl, wherein said phenyl and naphthyl substituents are each independently C₁-C₆ alkyl or C₁-C₆ alkoxy, R³⁸ is hydrogen, C₁-C₆ alkyl, amino, mono(C₁-C₆)alkylamino, di(C₁-C₆) alkylamino, phenylamino, diphenylamino, (mono- or di-(C₁-C₆)alkyl substituted phenyl)amino, (mono- or di-(C₁-C₆)alkoxy substituted phenyl)amino, di(mono- or di-(C₁-C₆)alkyl substituted phenyl)amino, di(mono- or di-(C₁-C₆)alkoxy substituted phenyl)amino, morpholino, piperidino or an unsubstituted, mono- or di-substituted phenyl or naphthyl, wherein said phenyl or naphthyl substituents are each independently C₁-C₆ alkyl or C₁-C₆ alkoxy, and R³⁹ is hydrogen, —C(═O)R³⁷, C₁-C₆ alkyl, C₁-C_{C}₃ alkoxy (C₁-C₆)alkyl, phenyl(C₁-C₆)alkyl, mono-alkoxy substituted phenyl(C₁-C₆)alkyl or an unsubstituted, mono- or di-substituted phenyl or naphthyl, wherein said phenyl or naphthyl substituents are each independently C₁-C₆ alkyl or C₁-C₆ alkoxy; provided that at least one of R¹³ and R¹⁴ is a perhalogenated group that is at least one of a perhalo(C₁-C₁₀)alkyl, a perhalo(C₂-C₁₀)alkenyl, a perhalo(C₃-C₁₀)alkynyl, a perhalo(C₁-C₁₀)alkoxy or a perhalo(C₃-C₁₀)cycloalkyl, or a group represented by —O(CH₂)ₐ(CX₂)_{b}CT₃;

B and B′ are each independently: a metallocenyl group; an aryl group that is mono-substituted with a reactive substituent or a compatiblizing substituent; 9-julolidinyl, an unsubstituted, mono-, di- or tri-substituted aryl group chosen from phenyl and naphthyl, an unsubstituted, mono- or di-substituted heteroaromatic group chosen from pyridyl, furanyl, benzofuran-2-yl, benzofuran-3-yl, thienyl, benzothien-2-yl, benzothien-3-yl, dibenzofuranyl, dibenzothienyl, carbazoyl, benzopyridyl, indolinyl or fluorenyl, wherein the aryl and heteroaromatic substituents are each independently: hydroxy, aryl, mono- or di-(C₁-C₁₂)alkoxyaryl, mono- or di-(C₁-C₁₂)alkylaryl, haloaryl, C₃-C₇ cycloalkylaryl, C₃-C₇ cycloalkyl, C₃-C₇ cycloalkyloxy, C₃-C₇ cycloalkyloxy(C₁-C₁₂)alkyl, C₃-C₇ cycloalkyloxy(C₁-C₁₂)alkoxy, aryl(C₁-C₁₂)alkyl, aryl(C₁-C₁₂)alkoxy, aryloxy, aryloxy(C₁-C₁₂)alkyl, aryloxy(C₁-C₁₂)alkoxy, mono- or di-(C₁-C₁₂)alkylaryl(C₁-C₁₂)alkyl, mono- or di-(C₁-C₁₂)alkoxyaryl(C₁-C₁₂)alkyl, mono- or di-(C₁-C₁₂)alkylaryl(C₁-C₁₂)alkoxy, mono- or di-(C₁-C₁₂)alkoxyaryl(C₁-C₁₂)alkoxy, amino, mono- or di-(C₁-C₁₂)alkylamino, diarylamino, piperazino, N—(C₁-C₁₂)alkylpiperazino, N-arylpiperazino, aziridino, indolino, piperidino, morpholino, thiomorpholino, tetrahydroquinolino, tetrahydroisoquinolino, pyrrolidino, C₁-C₁₂ alkyl, C₁-C₁₂ haloalkyl, C₁-C₁₂ alkoxy, mono(C₁-C₁₂)alkoxy(C₁-C₁₂)alkyl, acryloxy, methacryloxy, halogen or —C(═O)R¹⁵, wherein R¹⁵ is —OR¹⁶, —N(R¹⁷)R¹⁸, piperidino or morpholino, wherein R¹⁶ is allyl, C₁-C₆ alkyl, phenyl, mono(C₁-C₆)alkyl substituted phenyl, mono(C₁-C₆)alkoxy substituted phenyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl, C₁-C₆ alkoxy(C₂-C₄)alkyl or C₁-C₆ haloalkyl, and R¹⁷ and R¹⁸ are each independently C₁-C₆ alkyl, C₅-C₇ cycloalkyl or a substituted or an unsubstituted phenyl, wherein said phenyl substituents are each independently C₁-C₆ alkyl or C₁-C₆ alkoxy; an unsubstituted or mono-substituted group chosen from pyrazolyl, imidazolyl, pyrazolinyl, imidazolinyl, pyrrolidino, phenothiazinyl, phenoxazinyl, phenazinyl and acridinyl, wherein said substituents are each independently C₁-C₁₂ alkyl, C₁-C₁₂ alkoxy, phenyl or halogen; a 4-substituted phenyl, the substituent being a dicarboxylic acid residue or derivative thereof, a diamine residue or derivative thereof, an amino alcohol residue or derivative thereof, a polyol residue or derivative thereof, —(CH₂)—, —(CH₂)ₑ— or —[O—(CH₂)ₑ]_{f}—, wherein e is an integer ranging from 2 to 6 and f is an integer ranging from 1 to 50, and wherein the substituent is connected to an aryl group of another photochromic material; a group represented by:

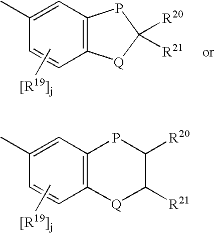

wherein P is —CH₂— or —O—; Q is —O— or substituted nitrogen, the substituted nitrogen substituents being hydrogen, C₁-C₁₂ alkyl or C₁-C₁₂ acyl, provided that when Q is substituted nitrogen, P is —CH₂—; each R¹⁹ is independently C₁-C₁₂ alkyl, C₁-C₁₂ alkoxy, hydroxy or halogen; R²⁰ and R²¹ are each independently hydrogen or C₁-C₁₂ alkyl; and j is an integer ranging from 0 to 2; or a group represented by:

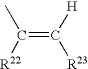

wherein R²² is hydrogen or C₁-C₁₂ alkyl; and R²³ is an unsubstituted, mono- or di-substituted naphthyl, phenyl, furanyl or thienyl, wherein said naphthyl, phenyl, furanyl and thienyl substituents are C₁-C₁₂ alkyl, C₁-C₁₂ alkoxy or halogen; or B and B′ taken together form a fluoren-9-ylidene or mono- or di-substituted fluoren-9-ylidene, wherein said fluoren-9-ylidene substituents are each independently C₁-C₁₂ alkyl, C₁-C₁₂ alkoxy or halogen;

R⁵, R⁸, R⁹ and R¹² are each independently: hydrogen, C₁-C₆ alkyl, chloro, fluoro, bromo, C₃-C₇ cycloalkyl or a unsubstituted, mono- or di-substituted phenyl, wherein said phenyl substituents are each independently C₁-C₆ alkyl or C₁-C₆ alkoxy; —OR⁴⁰ or —OC(═O)R⁴⁰, wherein R⁴⁰ is hydrogen, amine, alkylene glycol, polyalkylene glycol, C₁-C₆ alkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl, (C₁-C₆)alkoxy(C₂-C₄)alkyl, C₃-C₇ cycloalkyl, mono(C₁-C₄)alkyl substituted C₃-C₇ cycloalkyl or an unsubstituted, mono- or di-substituted phenyl, wherein said phenyl substituents are each independently C₁-C₆ alkyl or C₁-C₆ alkoxy; a reactive substituent or a compatiblizing substituent; a 4-substituted phenyl, the substituent being a dicarboxylic acid residue or derivative thereof, a diamine residue or derivative thereof, an amino alcohol residue or derivative thereof, a polyol residue or derivative thereof, —(CH₂)—, —(CH₂)ₑ— or —[O—(CH₂)ₑ]_{f}—, wherein e is an integer ranging from 2 to 6, and f is an integer ranging from 1 to 50, and wherein the substituent is connected to an aryl group of another photochromic material; —N(R⁴¹)R⁴², wherein R⁴¹ and R⁴² are each independently hydrogen, C₁-C₈ alkyl, phenyl, naphthyl, furanyl, benzofuran-2-yl, benzofuran-3-yl, thienyl, benzothien-2-yl, benzothien-3-yl, dibenzofuranyl, dibenzothienyl, benzopyridyl and fluorenyl, C₁-C₈ alkylaryl, C₃-C₈ cycloalkyl, C₄-C₁₆ bicycloalkyl, C₅-C₂₀ tricycloalkyl or C₁-C₂₀ alkoxy(C₁-C₆)alkyl, or R⁴¹ and R⁴² come together with the nitrogen atom to form a C₃-C₂₀ hetero-bicycloalkyl ring or a C₄-C₂₀ hetero-tricycloalkyl ring; a nitrogen containing ring represented by:

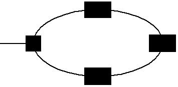

wherein each —V— is independently chosen for each occurrence from —CH₂—, —CH(R⁴³)—, —C(R⁴³)₂—, —CH(aryl)-, —C(aryl)₂— and —C(R⁴³)(aryl)-, wherein each R⁴³ is independently C₁-C₆ alkyl and each aryl is independently phenyl or naphthyl; —W— is —V—, —O—, —S—, —S(O)—, —SO₂—, —NH—, —N(R⁴³)— or —N(aryl)-; s is an integer ranging from 1 to 3; and r is an integer ranging from 0 to 3, provided that if r is 0, then —W— is the same as -V-; a group represented by:

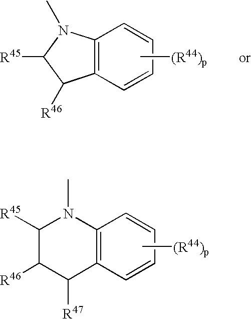

wherein each R⁴⁴ is independently C₁-C₆ alkyl, C₁-C₆ alkoxy, fluoro or chloro; R⁴⁵, R⁴⁶ and R⁴⁷ are each independently hydrogen, C₁-C₆ alkyl, phenyl or naphthyl, or R⁴⁵ and R⁴⁶ together form a ring of 5 to 8 carbon atoms, and p is an integer ranging from 0 to 3; or a substituted or an unsubstituted C₄-C₁₈ spirobicyclic amine or a substituted or an unsubstituted C₄-C₁₈ spirotricyclic amine, wherein said substituents are each independently aryl, C₁-C₆ alkyl, C₁-C₆ alkoxy or phenyl(C₁-C₆)alkyl;

R⁷ and R¹⁰ are each independently: any of the groups discussed above with respect to R⁵, R⁸, R⁹ and R¹²; or a metallocenyl group;

R⁶ and R¹¹ are each independently: any of the groups discussed above with respect to R⁷ and R¹⁰; perfluoroalkyl or perfluoroalkoxy; —C(═O)R⁴⁸ or -SO₂R⁴⁸, wherein R⁴⁸ is hydrogen, C₁-C₆ alkyl, —OR⁴⁹ or —NR⁵⁰R⁵¹, wherein R⁴⁹, R⁵⁰ and R⁵¹ are each independently hydrogen, C₁-C₆ alkyl, C₅-C₇ cycloalkyl, alkylene glycol, polyalkylene glycol or an unsubstituted, mono- or di-substituted phenyl, wherein said phenyl substituents are each independently C₁-C₆ alkyl or C₁-C₆ alkoxy; —C(═C(R⁵²)₂)R⁵³, and wherein each R⁵² is independently —C(═O)R⁴⁸, —OR⁴⁹, —OC(═O)R⁴⁹, —NR⁵⁰R⁵¹, hydrogen, halogen, cyano, C₁-C₆ alkyl, C₅-C₇ cycloalkyl, alkylene glycol, polyalkylene glycol or an unsubstituted, mono- or di-substituted phenyl, wherein said phenyl substituents are each independently C₁-C₆ alkyl or C₁-C₆ alkoxy; and R⁵³ is hydrogen, C₁-C₆ alkyl, C₅-C₇ cycloalkyl, alkylene glycol, polyalkylene glycol or an unsubstituted, mono- or di-substituted phenyl, wherein said phenyl substituents are each independently C₁-C₆ alkyl or C₁-C₆ alkoxy; or —C≡CR⁵⁴ or —C≡N, wherein R⁵⁴ is —C(═O)R⁴⁸, hydrogen, C₁-C₆ alkyl, C₅-C₇ cycloalkyl or an unsubstituted, mono- or di-substituted phenyl, wherein said phenyl substituents are each independently C₁-C₆ alkyl or C₁-C₆ alkoxy; or at least one pair of adjacent groups R⁶ and R⁷ or R¹⁰ and R¹¹ together form a group represented by:

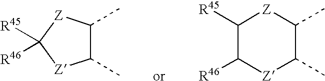

wherein Z and Z′ are each independently oxygen or the group —NR⁴¹—; or

R⁶ and R⁷ or R¹⁰ and R¹¹ together form an aromatic or heteroaromatic fused group, said fused group being benzo, indeno, dihydronaphthalene, indole, benzofuran, benzopyran or thianaphthene.

Still other non-limiting embodiments of the present invention relate to photochromic compositions, including photochromic coating compositions, and photochromic articles that comprise the photochromic materials and photochromic indeno[2′,3′:3,4]naphtho[1,2-b]pyrans according to various non-limiting embodiments disclosed herein and methods of making the same.

### BRIEF DESCRIPTION OF THE SEVERAL VIEWS OF THE DRAWINGS

Aspects of the present invention will be better understood when read in conjunction with the figures, in which:

FIG. 1 is a general reaction scheme for forming a substituted 7H-benzo[C]fluorenone that may be useful in forming photochromic materials according to various non-limiting embodiments disclosed herein;

FIGS. 2 and 3 are general reaction schemes for forming photochromic materials according to various non-limiting embodiments disclosed herein;

FIGS. 4a, 4b, and 4c show the unactivated and activated absorption spectra of UV light for the photochromic compounds of Examples 1, 7, and 11, respectively; and

FIGS. 5a, 5b, and 5c show the unactivated and activated absorption spectra of UV light for the photochromic compounds of Comparative Examples CE6, CE3, and CE5, respectively.

### DETAILED DESCRIPTION OF VARIOUS NON-LIMITING EMBODIMENTS OF THE INVENTION

Various non-limiting embodiments of the invention will now be described. It is to be understood that while the present invention is described herein in connection with certain embodiments and examples, the present invention is not limited to the particular embodiments and examples disclosed, but is intended to cover modifications that are within the spirit and scope of the invention, as defined by the appended claims. Further, it is to be understood that the present description illustrates aspects of the invention relevant to a clear understanding of the invention. Accordingly, certain aspects of the invention that would be apparent to those of ordinary skill in the art and that, therefore, would not facilitate a better understanding of the invention have not been presented in order to simplify the present description.

As used in this specification and the appended claims, the articles “a,” “an,” and “the” include plural referents unless expressly and unequivocally limited to one referent. Additionally, for the purposes of this specification, unless otherwise indicated, all numbers expressing quantities, such as, weight percentages and processing parameters, and other properties or parameters used in the specification are to be understood as being modified in all instances by the term “about.” Accordingly, unless otherwise indicated, it should be understood that the numerical parameters set forth in the following specification and attached claims are approximations. At the very least, and not as an attempt to limit the application of the doctrine of equivalents to the scope of the claims, numerical parameters should be read in light of the number of reported significant digits and the application of ordinary rounding techniques.

Further, while the numerical ranges and parameters setting forth the broad scope of the invention are approximations as discussed above, the numerical values set forth in the Examples section are reported as precisely as possible. It should be understood, however, that such numerical values inherently contain certain errors resulting from, for example, the measurement equipment and/or measurement technique. Furthermore, when numerical ranges are set forth herein, these ranges are inclusive of the recited range end point(s).

Moreover, it should be appreciated that where listings of possible substituent groups are provided herein using headings or sub-heading (e.g., (a), (b) . . . ; (1), (2) . . . ; (i) (ii) . . . ; etc), these headings or subheadings are provided only for convenience of reading and are not intended to limit the choice of substituents groups.

Photochromic materials according to various non-limiting embodiments of the invention will now be discussed. As used herein, the term “photochromic” means having an absorption spectrum for at least visible radiation that varies in response to at least actinic radiation. As used herein, the term “photochromic material” means any substance that is adapted to display photochromic properties, i.e. adapted to have an absorption spectrum for at least visible radiation that varies in response to at least actinic radiation. As discussed above, as used herein, the term “actinic radiation” refers to electromagnetic radiation that is capable of causing a photochromic material to transform from one form or state to another.

Example of photochromic materials include, without limitation, photochromic groups (e.g., indeno-fused naphthopyrans, etc.), as well as polymers, oligomers, monomers, and other compounds that comprise at least one photochromic group. As used herein, the term “group” means an arrangement of one or more atoms. Further, as used herein, the term “photochromic group” refers to an arrangement of atoms comprising a photochromic moiety. The term “moiety”, as used herein, means a part or portion of an organic molecule that has a characteristic chemical property. As used herein, the term “photochromic moiety” refers to the portion of a photochromic group that can undergo reversible transformation from one state to another upon exposure to actinic radiation.

The photochromic materials according to various non-limiting embodiments disclosed herein may comprise, in addition to a photochromic group, one or more other groups (e.g., functional groups, aliphatic groups, alicyclic groups, aromatic groups, heteroaromatic groups, heterocyclic groups, etc.) that are linked or fused to the photochromic group or another portion of the photochromic material. As used herein, the term “linked” means covalently bonded. Further, as used herein, the term “fused” means covalently bonded in at least two positions.

Various non-limiting embodiments of the present invention relate to a photochromic material comprising (i) an indeno[2′,3′:3,4]naphtho[1,2-b]pyran and (ii) a haloalkyl group bonded at the 13-position of the indeno[2′,3′:3,4]naphtho[1,2-b]pyran, wherein the haloalkyl group is (a) a perhalogenated group that is at least one of a perhalo(C₁-C₁₀)alkyl, a perhalo(C₂-C₁₀)alkenyl, a perhalo(C₃-C₁₀)alkynyl, a perhalo(C₁-C₁₀)alkoxy or a perhalo(C₃-C₁₀)cycloalkyl; or (b) a group represented by —O(CH₂)ₐ(CX₂)_{b}CT₃, wherein T independently represents halogen (e.g., fluorine, chlorine, bromine, etc.), X represents hydrogen or halogen (e.g., fluorine, chlorine, bromine, etc.), a represents an integer ranging from 1 to 10, and b represents an integer ranging from 1 to 10. In the present disclosure, the term “haloalkyl group” refers to a hydrocarbon group wherein at least one hydrogen atom is replaced with halogen atoms. As used herein, the terms “perhalogenated group” and “perhalo” refer to a hydrocarbon group wherein all the hydrogen atoms are replaced with halogen atoms.

As used herein, the term “indeno[2′,3′:3,4]naphtho[1,2-b]pyran” refers to a photochromic group that may be represented by the general structure (i) (below), and which comprises one or more group(s) bonded to the pyran ring at an available position adjacent to the oxygen atom (i.e., indicated as the groups B and B′ bonded at the 3-position in structure (i) below), which may aid in stabilizing the open-form of the indeno-fused naphthopyran. Non-limiting examples of groups that may be bonded to the pyran ring are described in more detail herein below with reference to the groups B and B′. As used herein, terms such as, “13-position,” “11-position,” “6-position,” etc. refer to the 13-, 11-, 6-positions, etc. (respectively) of the ring atoms of the indeno-fused naphthopyran as shown in structure (i).

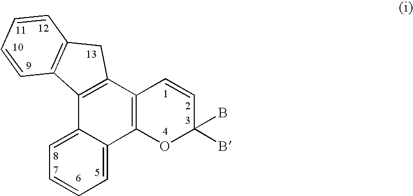

Further, it will be appreciated by those skilled in the art that any available position in the structure (i) may be substituted or unsubstituted as required. Non-limiting examples of groups that may be bonded to available positions on the indeno[2′,3′:3,4]naphtho[1,2-b]pyran according to various non-limiting embodiments disclose herein are set forth herein below in detail.

As discussed above, various non-limiting embodiments of the present invention relate to a photochromic material comprising an indeno[2′,3′:3,4]naphtho[1,2-b]pyran and a haloalkyl group bonded at the 13-position of the indeno[2′,3′:3,4]naphtho[1,2-b]pyran (i.e., the 13-positions of the ring atoms of the indeno-fused naphthopyran as shown in structure (i)), wherein the haloalkyl group is a perhalogenated group that is at least one of a perhalo(C₁-C₁₀)alkyl, a perhalo(C₂-C₁₀)alkenyl, a perhalo(C₃-C₁₀)alkynyl, a perhalo(C₁-C₁₀)alkoxy or a perhalo(C₃-C₁₀)cycloalkyl; or a group represented by —O(CH₂)ₐ(CX₂)_{b}CT₃, wherein T represents halogen, X represents hydrogen or halogen, a represents an integer ranging from 1 to 10, and b represents an integer ranging from 1 to 10. According to one specific non-limiting embodiment, the haloalkyl group may be a perhalo(C₁-C₁₀)alkyl represented by —C_{d}F_{(2d+1)}, wherein d represents an integer ranging from 1 to 10, and more specifically, wherein d is 1, i.e., forming a —CF₃ group.

According to another specific non-limiting embodiment, the haloalkyl group may be a group represented by —O(CH₂)ₐ(CX₂)_{b}CT₃, wherein T represents halogen, and X represents hydrogen or the same halogen group as T. For example, according to one non-limiting embodiment, T may be fluorine, X may be hydrogen or fluorine, a may be an integer ranging from 1 to 10, and b may be an integer ranging from 1 to 10. According to another non-limiting embodiment, the haloalkyl group may be a group represented by —O(CH₂)ₐ(CX₂)_{b}CT₃, wherein T represents halogen, X represents halogen, a is 1 or 2, and b is an integer ranging from 1 to 10.

Further, according to various non-limiting embodiments disclosed herein, the 13-position of the indeno[2′,3′:3,4]naphtho[1,2-b]pyran may be di-substituted with a first substituent and a second substituent, wherein the first substituent bonded at the 13-position of the indeno[2′,′:3,4]naphtho[1,2-b]pyran is one of the haloalkyl groups discussed above, and the second substituent bonded at the 13-position is one of the following groups (which may or may not be a haloalkyl group):

(a) a perhalogenated group that is at least one of a perhalo(C₁-C₁₀)alkyl, a perhalo(C₂-C₁₀)alkenyl, a perhalo(C₃-C₁₀)alkynyl, a perhalo(C₁-C₁₀)alkoxy or a perhalo(C₃-C₁₀)cycloalkyl;

(b) a group represented by —O(CH₂)ₐ(CX₂)_{b}CT₃, wherein T represents halogen, X represents hydrogen or halogen, a represents an integer ranging from 1 to 10, and b represents an integer ranging from 1 to 10;

(c) a silicon-containing group represented by one of

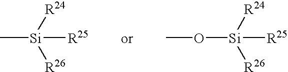

wherein R²⁴, R²⁵, and R²⁶ each independently represents a group, such as, C₁-C₁₀ alkyl, C₁-C₁₀ alkoxy or phenyl;

(d) a metallocenyl group;

(e) a reactive substituent or a compatiblizing substituent;

(f) hydrogen, hydroxy, C₁-C₆ alkyl, chloro, fluoro, C₃-C₇ cycloalkyl, allyl or C₁-C₈ haloalkyl;

(g) morpholino, piperidino, pyrrolidino, an unsubstituted, mono- or di-substituted amino, wherein said amino substituents are each independently C₁-C₆ alkyl, phenyl, benzyl or naphthyl;

(h) an unsubstituted, mono-, di- or tri-substituted aryl group chosen from phenyl, naphthyl, benzyl, phenanthryl, pyrenyl, quinoyl, isoquinolyl, benzofuranyl, thienyl, benzothienyl, dibenzofuranyl, dibenzothienyl, carbazolyl or indolyl, wherein the aryl group substituents are each independently halogen, C₁-C₆ alkyl or C₁-C₆ alkoxy;

(i) —C(═O)R²⁷, wherein R²⁷ represents a group, such as, hydrogen, hydroxy, C₁-C₆ alkyl, C₁-C₆ alkoxy, amino, mono- or di-(C₁-C₆)alkylamino, morpholino, piperidino, pyrrolidino, an unsubstituted, mono- or di-substituted phenyl or naphthyl, an unsubstituted, mono- or di-substituted phenoxy, an unsubstituted, mono- or di-substituted phenylamino, wherein said phenyl, naphthyl, phenoxy and phenylamino substituents are each independently C₁-C₆ alkyl or C₁-C₆ alkoxy;

(j) —OR²⁸, wherein R²⁸ represents a group, such as,
(i) C₁-C₆ alkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl, C₁-C₆ alkoxy(C₂-C₄)alkyl, C₃-C₇ cycloalkyl, mono(C₁-C₄)alkyl substituted C₃-C₇ cycloalkyl, C₁-C₈ chloroalkyl, C₁-C₈ fluoroalkyl, allyl or C₁-C₆ acyl,
(ii) —CH(R²⁹)R³⁰, wherein R²⁹ represents a group, such as, hydrogen or C₁-C₃ alkyl, R³⁰ represents a group, such as, —CN, —CF₃ or —COOR³¹, and R³¹ represents a group, such as, hydrogen or C₁-C₃ alkyl, or
(iii) —C(═O)R³², wherein R³² represents a group, such as, hydrogen, C₁-C₆ alkyl, C₁-C₆ alkoxy, amino, mono- or di-(C₁-C₆)alkylamino, an unsubstituted, mono- or di-substituted phenyl or naphthyl, an unsubstituted, mono- or di-substituted phenoxy or an unsubstituted, mono- or di-substituted phenylamino, wherein said phenyl, naphthyl, phenoxy and phenylamino substituents are each independently C₁-C₆ alkyl or C₁-C₆ alkoxy;

(k) a 4-substituted phenyl, the substituent being a dicarboxylic acid residue or derivative thereof, a diamine residue or derivative thereof, an amino alcohol residue or derivative thereof, a polyol residue or derivative thereof, —(CH₂)—, —(CH₂)ₑ— or —[O—(CH₂)ₑ]_{f}—, wherein e represents an integer ranging from 2 to 6 and f represents an integer ranging from 1 to 50, and wherein the substituent is connected to an aryl group of another photochromic material;

(l) —CH(R³³)₂, wherein R³³ represents a group, such as, —CN or —COOR³⁴, wherein R³⁴ represents a group, such as, hydrogen, C₁-C₆ alkyl, C₃-C₇ cycloalkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl or an unsubstituted, mono- or di-substituted phenyl or naphthyl, wherein said phenyl and naphthyl substituents are each independently C₁-C₆ alkyl or C₁-C₆ alkoxy; or

(m) —CH(R³⁵)R³⁶, wherein R³⁵ represents a group, such as, hydrogen, C₁-C₆ alkyl or an unsubstituted, mono- or di-substituted phenyl or naphthyl, wherein said phenyl and naphthyl substituents are each independently C₁-C₆ alkyl or C₁-C₆ alkoxy, and R³⁶ represents a group, such as, —C(═O)OR³⁷, —C(═O)R³⁸ or —CH₂OR³⁹ wherein:
(i) R³⁷ represents a group, such as, hydrogen, C₁-C₆ alkyl, C₃-C₇ cycloalkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl or an unsubstituted, mono- or di-substituted aryl groups phenyl or naphthyl, wherein said phenyl and naphthyl substituents are each independently C₁-C₆ alkyl or C₁-C₆ alkoxy,
(ii) R³⁸ represents a group, such as, hydrogen, C₁-C₆ alkyl, amino, mono(C₁-C₆)alkylamino, di(C₁-C₆) alkylamino, phenylamino, diphenylamino, (mono- or di-(C₁-C₆)alkyl substituted phenyl)amino, (mono- or di-(C₁-C₆)alkoxy substituted phenyl)amino, di(mono- or di-(C₁-C₆)alkyl substituted phenyl)amino, di(mono- or di-(C₁-C₆)alkoxy substituted phenyl)amino, morpholino, piperidino or an unsubstituted, mono- or di-substituted phenyl or naphthyl, wherein said phenyl or naphthyl substituents are each independently C₁-C₆ alkyl or C₁-C₆ alkoxy, and
(iii) R³⁹ represents a group, such as, hydrogen, —C(═O)R³⁷ (groups that R³⁷ may represent are set forth above), C₁-C₆ alkyl, C₁-C₃ alkoxy (C₁-C₆)alkyl, phenyl(C₁-C₆)alkyl, mono-alkoxy substituted phenyl(C₁-C₆)alkyl or an unsubstituted, mono- or di-substituted phenyl or naphthyl, wherein said phenyl or naphthyl substituents are each independently C₁-C₆ alkyl or C₁-C₆ alkoxy.

According to various non-limiting embodiments wherein the 13-position of the indeno[2′,3′:3,4]naphtho[1,2-b]pyran is di-substituted as discussed above, the second substituent that is bonded at the 13-position may be a haloalkyl group that is the same as the first substituent.

According to various other non-limiting embodiments wherein the 13-position of the indeno[2′,3′:3,4]naphtho[1,2-b]pyran is di-substituted as discussed above, the second substituent may be a silicon-containing group represented by:

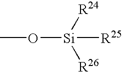

wherein at least two of R²⁴, R²⁵ and R²⁶ independently represent C₁-C₁₀ alkyl or phenyl. According to one specific non-limiting embodiment, at least two of R²⁴, R²⁵ and R²⁶ may be methyl, for example, forming a trimethylsiloxy group or a t-butyldimethylsiloxy group. In yet another specific non-limiting embodiment, at least two of R²⁴, R²⁵ and R²⁶ may be phenyl, for example, forming a t-butyldiphenylsiloxy group.

Further, according to various non-limiting embodiments disclosed herein, the photochromic material may comprise an indeno[2′,3′:3,4]naphtho[1,2-b]pyran, a first substituent bonded at the 13-position of the indeno[2′,3′:3,4]naphtho[1,2-b]pyran, the first substituent being a perhalo(C₁-C₁₀)alkyl group represented by —C_{d}F_{(2d+1)}, wherein d represents an integer ranging from 1 to 10, and a second substituent bonded at the 13-position of the indeno[2′,3′:3,4]naphtho [1,2-b]pyran, the second substituent being a silicon-containing group represented by:

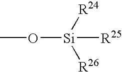

wherein R²⁴, R²⁵ and R²⁶ each independently represent C₁-C₁₀ alkyl or phenyl. For example, although not limiting herein, according to one non-limiting embodiment, d may be 1 (i.e., the group —CF₃) and at least two of R²⁴, R²⁵ and R²⁶ may be methyl (e.g., a trimethylsiloxy group or a t-butyldimethylsiloxy group) or phenyl (e.g., a t-butyldiphenylsiloxy group).

As indicated above, according to various non-limiting embodiments disclosed herein, the second substituent bonded at the 13-position of the indeno[2′,3′:3,4]naphtho[1,2-b]pyran may be a metallocenyl group. As used herein, the term “metallocene group” refers to a group in which two cyclopentadienyl ring ligands form a “sandwich” around a metal ion, wherein each cyclopentadienyl ring is bonded to the metal ion by a pentahapto (η⁵) bonding structure. Metallocene groups have the general empirical formula (C₅H₅)₂M, where M is a metal ion having a +2 oxidation state. As used herein, the term “metallocenyl group” refers to a metallocene group that forms or is capable of forming at least one bond with at least one other group, such as, for example, a photochromic group. Specific, non-limiting examples of metallocenyl groups that may be used in connection with the photochromic materials according to various non-limiting embodiments disclosed herein include: ferrocenyl groups, titanocenyl groups, ruthenocenyl groups, osmocenyl groups, vanadocenyl groups, chromocenyl groups, cobaltocenyl groups, nickelocenyl groups, and di-π-cyclopentadienyl-manganese groups. According to one specific non-limiting embodiment, the metallocenyl group that is bonded to the indeno-fused naphthopyran at the 13-position is a ferrocenyl group.

According to various non-limiting embodiments disclosed herein wherein the photochromic material comprises a metallocenyl group (which may be bonded to the indeno-fused naphthopyran at the 13-position as discussed above, or another available position as discussed herein below), the metallocenyl group may be further substituted. For example, according to various non-limiting embodiments disclosed herein, the metallocenyl group may be represented by one of the following general structures (ii) or (iii) (wherein the dashed line represents an attachment to an indeno-fused naphthopyran, either directly or through a tether, such as, a C₁-C₆ alkyl, C₁-C₆ alkoxy, or polyalkylene glycol tether):

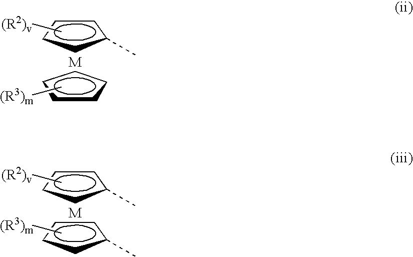

wherein M represents Ti, V, Cr, Mn, Fe, Ru, Os, Co or Ni; v and m each represent an integer from 0 to 3, each R² independently represents a group, such as, halogen, C₁-C₃ alkyl, phenyl(C₁-C₃) alkyl, C₁-C₃ alkoxy, phenyl(C₁-C₃) alkoxy, amino, vinyl or the group —C(O)R⁴ wherein R⁴ represents a group, such as, hydrogen, hydroxy, C₁-C₃ alkyl, phenyl; or two adjacent R² substituent groups may together form a benzo group; and each R³ may independently represent a group, such as, another photochromic group (for example, another indeno-fused naphthopyran, attached either directly or through a tether, as described above) or any group discussed above with respect to R². According to one non-limiting embodiment, M represents Ti, Cr, Fe or Ru. According to another non-limiting embodiment, M represents Fe. Non-limiting examples of photochromic materials and methods of making photochromic materials comprising a metallocenyl group that may be suitable for use in connection with various non-limiting embodiments of the present invention are disclosed at paragraphs [0019] to [0063] of U.S. application Ser. No. 11/443,938, entitled “Photochromic Materials Comprising Metallocenyl Groups”, which was filed on a date even herewith, and which disclosure is hereby specifically incorporated by reference herein.

As indicated above and discussed in more detail herein below, the photochromic materials according to various non-limiting embodiments disclosed herein may further comprise a reactive substituent or a compatiblizing substituent. As used herein, the term “reactive substituent” means an arrangement of atoms, wherein a portion of the arrangement comprises a reactive moiety or a residue thereof. As used herein, the term “reactive moiety” means a part or portion of an organic molecule that may react to form one or more bond(s) with a monomer, an intermediate in a polymerization reaction or with a polymer into which it has been incorporated. As used herein, the term “intermediate in a polymerization reaction” means any combination of two or more monomer units that are capable of reacting to form one or more bond(s) to additional monomer unit(s) to continue a polymerization reaction or, alternatively, reacting with a reactive moiety of the reactive substituent on the photochromic material. For example, although not limiting herein, the reactive moiety may react with an intermediate in a polymerization reaction of a monomer or oligomer as a co-monomer in the polymerization reaction or may react as, for example and without limitation, a nucleophile or electrophile that adds into the intermediate. Alternatively, the reactive moiety may react with a group (such as, but not limited to a hydroxyl group) on a polymer. Further, the reactive moiety can be reacted with a protecting group. As used herein, the term “protecting group” means a group that is removably bonded to a reactive moiety that prevents the reactive moiety from participating in a reaction until the group is removed. Optionally, the reactive substituents according to various non-limiting embodiments disclosed herein may further comprise a linking group. As used herein, the term “linking group” means one or more group(s) or chain(s) of atoms that connect the reactive moiety to the photochromic material.

As used herein, the term “compatiblizing substituent” means an arrangement of atoms that can facilitate integration of the photochromic material into another material or solvent. For example, according to various non-limiting embodiments disclosed herein, the compatiblizing substituent may facilitate integration of the photochromic material into a hydrophilic material by increasing the miscibility of the photochromic material in water or a hydrophilic polymeric, oligomeric or monomeric material. According to other non-limiting embodiments, the compatiblizing substituent may facilitate integration of the photochromic material into a lipophilic material. Although not limiting herein, photochromic materials according to various non-limiting embodiments disclosed herein that comprise a compatiblizing substituent that facilitates integration into a hydrophilic material may be miscible in hydrophilic material at least to the extent of one gram per liter. Non-limiting examples of compatiblizing substituents include those substituents comprising a group -J, wherein -J represents the group —K (discussed below) or hydrogen.

Further, it should be appreciated that some substituents may be both a compatiblizing substituent and a reactive substituent. For example, a substituent that comprises hydrophilic linking group(s) that connects a reactive moiety to the photochromic material may be both a reactive substituent and a compatiblizing substituent. As used herein, such substituents may be termed as either a reactive substituent or a compatiblizing substituent.

Non-limiting examples of reactive and/or compatiblizing substituents that may be used in conjunction with the various non-limiting embodiments disclosed herein may be represented by:
-A-D-E-G-J (v); -G-E-G-J (vi); -D-E-G-J (vii);
-A-D-J (viii); -D-G-J (ix); -D-J (x);
-A-G-J (xi); -G-J (xii); or -A-J (xiii).

With reference to (v)-(xiii) above, non-limiting examples of groups that -A- may represent according to various non-limiting embodiments disclosed herein include —O—, —C(═O)—, —CH₂—, —OC(═O)— and —NHC(═O)—, provided that if -A- represents —O—, -A- forms at least one bond with -J. Non-limiting examples of groups that -D- may represent according to various non-limiting embodiments include: (a) a diamine residue or a derivative thereof, wherein a first amino nitrogen of said diamine residue may form a bond with -A-, or a substituent or an available position on the indeno-fused naphthopyran, and a second amino nitrogen of said diamine residue may form a bond with -E-, -G- or -J; and (b) an amino alcohol residue or a derivative thereof, wherein an amino nitrogen of said amino alcohol residue may form a bond with -A-, or a substituent or an available position on the indeno-fused naphthopyran, and an alcohol oxygen of said amino alcohol residue may form a bond with -E-, -G- or -J. Alternatively, according to various non-limiting embodiments disclosed herein the amino nitrogen of said amino alcohol residue may form a bond with -E-, -G- or -J, and said alcohol oxygen of said amino alcohol residue may form a bond with -A-, or a substituent or an available position on the indeno-fused naphthopyran.

Non-limiting examples of suitable diamine residues that -D- may represent include an aliphatic diamine residue, a cyclo aliphatic diamine residue, a diazacycloalkane residue, an azacyclo aliphatic amine residue, a diazacrown ether residue, or an aromatic diamine residue. Specific non-limiting examples of diamine residues include the following:

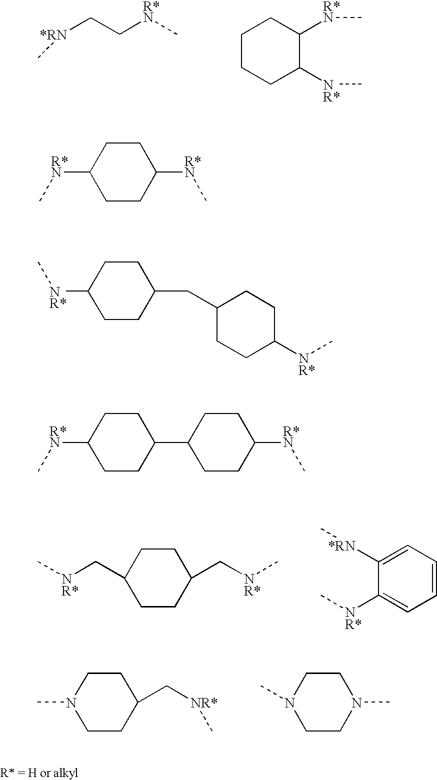

Non-limiting examples of suitable amino alcohol residues that -D- may represent include an aliphatic amino alcohol residue, a cyclo aliphatic amino alcohol residue, an azacyclo aliphatic alcohol residue, a diazacyclo aliphatic alcohol residue or an aromatic amino alcohol residue. Specific non-limiting examples of amino alcohol residues that may be used in conjunction with various non-limiting embodiments disclosed herein include the following:

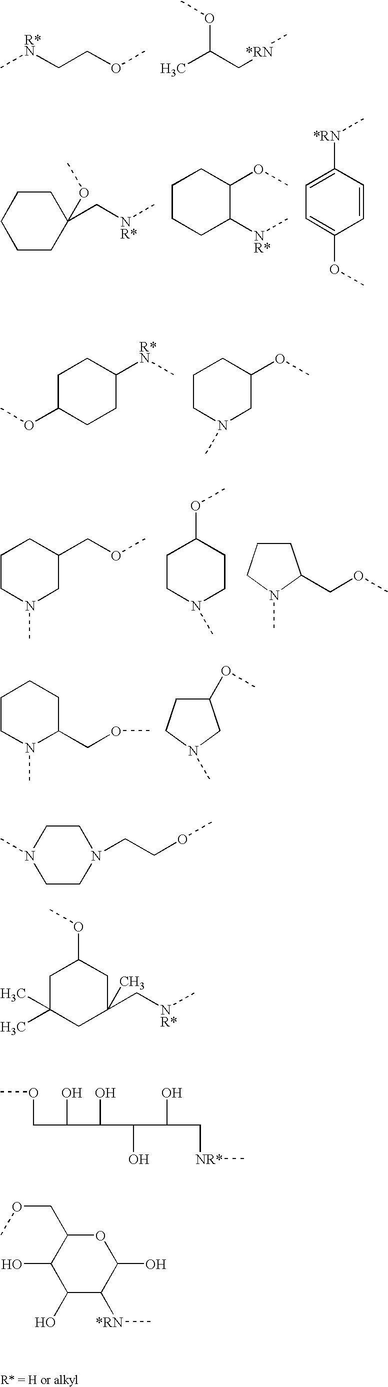

With continued reference to (v)-(xiii) above, according to various non-limiting embodiments disclosed herein, -E- may represent a dicarboxylic acid residue or a derivative thereof, wherein a first carbonyl group of said dicarboxylic acid residue may form a bond with -G- or -D-, and a second carbonyl group of said dicarboxylic acid residue may form a bond with -G-. Non-limiting examples of suitable dicarboxylic acid residues that -E- may represent include an aliphatic dicarboxylic acid residue, a cycloaliphatic dicarboxylic acid residue or an aromatic dicarboxylic acid residue. Specific non-limiting examples of dicarboxylic acid residues that may be used in conjunction with various non-limiting embodiments disclosed herein include the following:

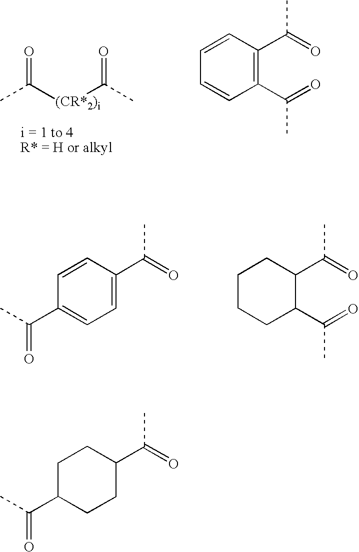

According to various non-limiting embodiments disclosed herein, -G- may represent: (a) a group —[(OC₂H₄)ₓ(OC₃H₆)_{y}(OC₄H₈)_{z}]—O—, wherein x, y and z are integers that are each independently chosen and range from 0 to 50, and a sum of x, y and z ranges from 1 to 50; (b) a polyol residue or a derivative thereof, wherein a first polyol oxygen of said polyol residue may form a bond with -A-, -D-, -E- or a substituent or an available position on the indeno-fused naphthopyran and a second polyol oxygen of said polyol may form a bond with -E- or -J; or (c) a combination of (a) and (b), wherein the first polyol oxygen of the polyol residue forms a bond with a group —[(OC₂H₄)ₓ(OC₃H₆)_{y}(OC₄H₈)_{z}]— (i.e., to form the group —[(OC₂H₄)ₓ(OC₃H₆)_{y}(OC₄H₈)_{z}]—O—), and the second polyol oxygen forms a bond with -E- or -J. Non-limiting examples of suitable polyol residues that -G- may represent include an aliphatic polyol residue, a cyclo aliphatic polyol residue or an aromatic polyol residue.

Specific non-limiting examples of polyols from which the polyol residues that -G- may represent may be formed according to various non-limiting embodiments disclosed herein include: (a) low molecular weight polyols having an average molecular weight less than 500, such as, but not limited to, those set forth in U.S. Pat. No. 6,555,028 at col. 4, lines 48-50, and col. 4, line 55 to col. 6, line 5, which disclosure is hereby specifically incorporated by reference herein; (b) polyester polyols, such as, but not limited to, those set forth in U.S. Pat. No. 6,555,028 at col. 5, lines 7-33, which disclosure is hereby specifically incorporated by reference herein; (c) polyether polyols, such as, but not limited to, those set forth in U.S. Pat. No. 6,555,028 at col. 5, lines 34-50, which disclosure is hereby specifically incorporated by reference herein; (d) amide-containing polyols, such as, but not limited to, those set forth in U.S. Pat. No. 6,555,028 at col. 5, lines 51-62, which disclosure is hereby specifically incorporated by reference; (e) epoxy polyols, such as, but not limited to, those set forth in U.S. Pat. No. 6,555,028 at col. 5 line 63 to col. 6, line 3, which disclosure is hereby specifically incorporated by reference herein; (f) polyhydric polyvinyl alcohols, such as, but not limited to, those set forth in U.S. Pat. No. 6,555,028 at col. 6, lines 4-12, which disclosure is hereby specifically incorporated by reference herein; (g) urethane polyols, such as, but not limited to, those set forth in U.S. Pat. No. 6,555,028 at col. 6, lines 13-43, which disclosure is hereby specifically incorporated by reference herein; (h) polyacrylic polyols, such as, but not limited to, those set forth in U.S. Pat. No. 6,555,028 at col. 6, lines 43 to col. 7, line 40, which disclosure is hereby specifically incorporated by reference herein; (i) polycarbonate polyols, such as, but not limited to, those set forth in U.S. Pat. No. 6,555,028 at col. 7, lines 41-55, which disclosure is hereby specifically incorporated by reference herein; and (j) mixtures of such polyols.

Referring again to (v)-(xiii) above, according to various non-limiting embodiments disclosed herein -J may represent a group —K, wherein —K represents a group, such as, but not limited to, —CH₂COOH, —CH(CH₃)COOH, —C(O)(CH₂)_{w}COOH, —C₆H₄SO₃H, —C₅H₁₀SO₃H, —C₄H₈SO₃H, —C₃H₆SO₃H, —C₂H₄SO₃H and —SO₃H, wherein w represents an integer ranging from 1 to 18. According to other non-limiting embodiments, -J may represent hydrogen that forms a bond with an oxygen or a nitrogen of a linking group to form a reactive moiety, such as, —OH or —NH. For example, according to various non-limiting embodiments disclosed herein, -J may represent hydrogen, provided that if -J represents hydrogen, -J is bonded to an oxygen of -D- or -G-, or a nitrogen of -D-.

According to still other non-limiting embodiments, -J may represent a group -L or residue thereof, wherein -L may represent a reactive moiety. For example, according to various non-limiting embodiments disclosed herein, -L may represent a group, such as, but not limited to, acryl, methacryl, crotyl, 2-(methacryloxy)ethylcarbamyl, 2-(methacryloxy)ethoxycarbonyl, 4-vinylphenyl, vinyl, 1-chlorovinyl or epoxy. As used herein, the terms acryl, methacryl, crotyl, 2-(methacryloxy)ethylcarbamyl, 2-(methacryloxy)ethoxycarbonyl, 4-vinylphenyl, vinyl, 1-chlorovinyl, and epoxy refer to the following structures:

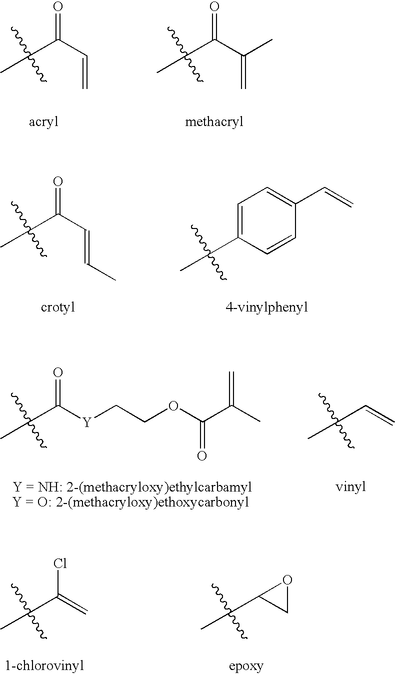

As previously discussed, -G- may represent a residue of a polyol, which is defined herein to include hydroxy-containing carbohydrates, such as, those set forth in U.S. Pat. No. 6,555,028 at col. 7, line 56 to col. 8, line 17, which disclosure is hereby specifically incorporated by reference herein. The polyol residue may be formed, for example and without limitation herein, by the reaction of one or more of the polyol hydroxyl groups with a precursor of -A-, such as, a carboxylic acid or a methylene halide, a precursor of polyalkoxylated group, such as, polyalkylene glycol, or a hydroxyl substituent of the indeno-fused naphthopyran. The polyol may be represented by R′—(OH)_{g} and the residue of the polyol may be represented by the formula —O—R′—(OH)_{g-1}, wherein R′ is the backbone or main chain of the polyhydroxy compound and g is at least 2.

Further, as discussed above, one or more of the polyol oxygens of -G- may form a bond with -J (i.e., forming the group -G-J). For example, although not limiting herein, wherein the reactive and/or compatiblizing substituent comprises the group -G-J, if -G- represents a polyol residue and -J represents a group —K that contains a carboxyl terminating group, -G-J may be produced by reacting one or more polyol hydroxyl groups to form the group —K (for example, as discussed with respect to Reactions B and C at col. 13, line 22 to col. 16, line 15 of U.S. Pat. No. 6,555,028, which disclosure is hereby specifically incorporated by reference herein) to produce a carboxylated polyol residue. Alternatively, if -J represents a group —K that contains a sulfo or sulfono terminating group, although not limiting herein, -G-J may be produced by the acidic condensation of one or more of the polyol hydroxyl groups with HOC₆H₄SO₃H; HOC₅H₁₀SO₃H; HOC₄H₈SO₃H; HOC₃H₆SO₃H; HOC₂H₄SO₃H; or H₂SO₄, respectively. Further, although not limiting herein, if -G- represents a polyol residue and -J represents a group -L chosen from acryl, methacryl, 2-(methacryloxy)ethylcarbamyl and epoxy, -L may be added by condensation of the polyol residue with acryloyl chloride, methacryloyl chloride, 2-isocyanatoethyl methacrylate or epichlorohydrin, respectively.

Further, although not limiting herein, wherein the photochromic material comprises two or more reactive substituents, two or more compatiblizing substituents, or a combination of reactive substituents and compatiblizing substituents, each substituent may be the same or different and may be independently chosen from those reactive and/or compatiblizing substituents discussed above. Additional examples of reactive and/or compatiblizing substituents and information regarding methods of forming such substituents on photochromic materials are provided at paragraphs [0051] to [0067] of U.S. patent application Ser. No. 11/102,279; U.S. patent application Ser. No. 11/102,280, at paragraphs [0017] to [0045]; U.S. Pat. No. 6,555,028, at col. 3, line 45 to col. 4, line 26; and U.S. Pat. No. 6,113,814 at col. 3, lines 30-64, which disclosures are hereby specifically incorporated by reference herein.

The photochromic materials according to various non-limiting embodiments disclosed herein may be represented by the structure (iv) shown below:

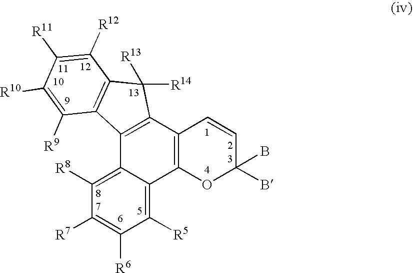

wherein at least one of R¹³ and R¹⁴ represents a haloalkyl group that is at least one of: (a) perhalogenated group that is at least one of a perhalo(C₁-C¹⁰)alkyl, a perhalo(C₂-C₁₀)alkenyl, a perhalo(C₃-C₁₀)alkynyl, a perhalo(C₁-C₁₀)alkoxy or a perhalo(C₃-C₁₀)cycloalkyl; or (b) a group represented by —O(CH₂)ₐ(CX₂)_{b}CT₃, wherein T is a halogen, X is hydrogen or halogen, a is an integer ranging from 1 to 10, and b is an integer ranging from 1 to 10. Suitable non-limiting examples of groups that B, B′ and R⁵-R¹² may represent, as well as other groups that R¹³ and R¹⁴ may represent according to various non-limiting embodiments disclosed herein are set forth below in more detail.

Non-limiting examples of groups that R¹³ or R¹⁴ may represent, in addition to the perhalogenated groups and the groups represented by —O(CH₂)ₐ(CX₂)_{b}CT₃ discussed above, include:

(a) a metallocenyl group (such as those discussed above);

(b) a reactive substituent or a compatiblizing substituent (such as those discussed above);

(c) a silicon-containing group represented by one of

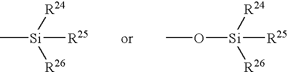

wherein R²⁴, R²⁵, and R²⁶ each independently represent a group, such as, C₁-C₁₀ alkyl, C₁-C₁₀ alkoxy or phenyl;

(d) hydrogen, hydroxy, C₁-C₆ alkyl, chloro, fluoro, C₃-C₇ cycloalkyl, allyl or C₁-C₈ haloalkyl;

(e) morpholino, piperidino, pyrrolidino, an unsubstituted, mono- or di-substituted amino, wherein said amino substituents are each independently C₁-C₆ alkyl, phenyl, benzyl or naphthyl;

(f) an unsubstituted, mono- di- or tri-substituted aryl group chosen from phenyl, naphthyl, benzyl, phenanthryl, pyrenyl, quinoyl, isoquinolyl, benzofuranyl, thienyl, benzothienyl, dibenzofuranyl, dibenzothienyl, carbazolyl and indolyl, wherein said aryl group substituents are each independently halogen, C₁-C₆ alkyl or C₁-C₆ alkoxy;

(g) —C(═O)R²⁷, wherein R²⁷ represents a group, such as, hydrogen, hydroxy, C₁-C₆ alkyl, C₁-C₆ alkoxy, amino, mono- or di-(C₁-C₆)alkylamino, morpholino, piperidino, pyrrolidino, an unsubstituted, mono- or di-substituted phenyl or naphthyl, an unsubstituted, mono- or di-substituted phenoxy, an unsubstituted, mono- or di-substituted phenylamino, wherein said phenyl, naphthyl, phenoxy, and phenylamino substituents are each independently C₁-C₆ alkyl or C₁-C₆ alkoxy;

(h) —OR²⁸, wherein R²⁸ represents a group, such as,
(i) C₁-C₆ alkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl, C₁-C₆ alkoxy(C₂-C₄)alkyl, C₃-C₇ cycloalkyl, mono(C₁-C₄)alkyl substituted C₃-C₇ cycloalkyl, C₁-C₈ chloroalkyl, C₁-C₈ fluoroalkyl, allyl or C₁-C₆ acyl,
(ii) —CH(R²⁹)R³⁰, wherein R³² represents a group, such as, hydrogen or C₁-C₃ alkyl, and R³⁰ represents a group, such as, —CN, —CF₃ or —COOR³ , wherein R³¹ represents a group, such as, hydrogen or C₁-C₃ alkyl, or
(iii) —C(═O)R³², wherein R³² represents a group, such as, hydrogen, C₁-C₆ alkyl, C₁-C₆ alkoxy, amino, mono- or di-(C₁-C₆)alkylamino, an unsubstituted, mono- or di-substituted phenyl or naphthyl, an unsubstituted, mono- or di-substituted phenoxy or an unsubstituted, mono- or di-substituted phenylamino, wherein said phenyl, naphthyl, phenoxy and phenylamino substituents are each independently C₁-C₆ alkyl or C₁-C₆ alkoxy;

(i) a 4-substituted phenyl, the substituent being a dicarboxylic acid residue or derivative thereof, a diamine residue or derivative thereof, an amino alcohol residue or derivative thereof, a polyol residue or derivative thereof, —(CH₂)—, —(CH₂)ₑ— or —┌O—(CH₂)ₑ┐_{f}—, wherein e represents an integer ranging from 2 to 6 and f represents an integer ranging from 1 to 50, and wherein the substituent is connected to an aryl group of another photochromic material (e.g., an aryl group of an indeno-fused naphthopyran);

(j) —CH(R³³)₂, wherein R³³ represents a group, such as, —CN or —COOR³⁴, wherein R³⁴ represents a group, such as, hydrogen, C₁-C₆ alkyl, C₃-C₇ cycloalkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl or an unsubstituted, mono- or di-substituted phenyl or naphthyl, wherein said phenyl and naphthyl substituents are each independently C₁-C₆ alkyl or C₁-C₆ alkoxy; or

(k) —CH(R³⁵)R³⁶, wherein R³⁵ represents a group, such as, hydrogen, C₁-C₆ alkyl or an unsubstituted, mono- or di-substituted phenyl or naphthyl, wherein said phenyl or naphthyl substituents are each independently C₁-C₆ alkyl or C₁-C₆ alkoxy, and R³⁶ represents a group, such as, —C(═O)OR³⁷, —C(═O)R³⁸ or —CH₂OR³⁹ wherein
(i) R³⁷ represents a group, such as, hydrogen, C₁-C₆ alkyl, C₃-C₇ cycloalkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl or an unsubstituted, mono- or di-substituted phenyl or naphthyl, wherein said phenyl and naphthyl substituents are each independently C₁-C₆ alkyl or C₁-C₆ alkoxy,
(ii) R³⁸ represents a group, such as, hydrogen, C₁-C₆ alkyl, amino, mono(C₁-C₆)alkylamino, di(C₁-C₆) alkylamino, phenylamino, diphenylamino, (mono- or di-(C₁-C₆)alkyl substituted phenyl)amino, (mono- or di-(C₁-C₆)alkoxy substituted phenyl)amino, di(mono- or di-(C₁-C₆)alkyl substituted phenyl)amino, di(mono- or di-(C₁-C₆)alkoxy substituted phenyl)amino, morpholino, piperidino or an unsubstituted, mono- or di-substituted phenyl or naphthyl, wherein said phenyl and naphthyl substituents are each independently C₁-C₆ alkyl or C₁-C₆ alkoxy, and
(iii) R³⁹ represents a group, such as, hydrogen, —C(═O)R³⁷ (examples of groups that R³⁷ may represent are set forth above), C₁-C₆ alkyl, C₁-C₃ alkoxy (C₁-C₆)alkyl, phenyl(C₁-C₆)alkyl, mono-alkoxy substituted phenyl(C₁-C₆)alkyl or an unsubstituted, mono- or di-substituted phenyl or naphthyl, wherein said phenyl and naphthyl substituents are each independently C₁-C₆ alkyl or C₁-C₆ alkoxy.

With continued reference to structure (iv) above, non-limiting examples of groups that B and B′ may each independently represent include:

(a) a metallocenyl group (such as those discussed above);

(b) an aryl group that is mono-substituted with a reactive substituent or a compatiblizing substituent(such as those discussed above);

(c) 9-julolidinyl, an unsubstituted, mono-, di- or tri-substituted aryl group chosen from phenyl and naphthyl, an unsubstituted, mono- or di-substituted heteroaromatic group chosen from pyridyl, furanyl, benzofuran-2-yl, benzofuran-3-yl, thienyl, benzothien-2-yl, benzothien-3-yl, dibenzofuranyl, dibenzothienyl, carbazoyl, benzopyridyl, indolinyl and fluorenyl, wherein the aryl and heteroaromatic substituents are each independently:
hydroxy, aryl, mono- or di-(C₁-C₁₂)alkoxyaryl, mono- or di-(C₁-C₁₂)alkylaryl, haloaryl, C₃-C₇ cycloalkylaryl, C₃-C₇ cycloalkyl, C₃-C₇ cycloalkyloxy, C₃-C₇ cycloalkyloxy(C₁-C₁₂)alkyl, C₃-C₇ cycloalkyloxy(C₁-C₁₂)alkoxy, aryl(C₁-C₁₂)alkyl, aryl(C₁-C₁₂)alkoxy, aryloxy, aryloxy(C₁-C₁₂)alkyl, aryloxy(C₁-C₁₂)alkoxy, mono- or di-(C₁-C₁₂)alkylaryl(C₁-C₁₂)alkyl, mono- or di-(C₁-C₁₂)alkoxyaryl(C₁-C₁₂)alkyl, mono- or di-(C₁-C₁₂)alkylaryl(C₁-C₁₂)alkoxy, mono- or di-(C₁-C₁₂)alkoxyaryl(C₁-C₁₂)alkoxy, amino, mono- or di-(C₁-C₁₂)alkylamino, diarylamino, piperazino, N—(C₁-C₁₂)alkylpiperazino, N-arylpiperazino, aziridino, indolino, piperidino, morpholino, thiomorpholino, tetrahydroquinolino, tetrahydroisoquinolino, pyrrolidino, C₁-C₁₂ alkyl, C₁-C₁₂ haloalkyl, C₁-C₁₂ alkoxy, mono(C₁-C₁₂ )alkoxy(C₁-C₁₂ )alkyl, acryloxy, methacryloxy, halogen or —C(═O)R¹⁵, wherein R¹⁵ represents a group, such as, —OR¹⁶, —N(R¹⁷)R¹⁸, piperidino or morpholino, wherein R¹⁶ represents a group, such as, allyl, C₁-C₆ alkyl, phenyl, mono(C₁-C₆)alkyl substituted phenyl, mono(C₁-C₆)alkoxy substituted phenyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl, C₁-C₆ alkoxy(C₂-C₄)alkyl or C₁-C₆ haloalkyl, and R¹⁷ and R¹⁸ each independently represents a group, such as, C₁-C₆ alkyl, C₅-C₇ cycloalkyl or a substituted or an unsubstituted phenyl, wherein said phenyl substituents are each independently C₁-C₆ alkyl or C₁-C₆ alkoxy;

(d) an unsubstituted or mono-substituted group chosen from pyrazolyl, imidazolyl, pyrazolinyl, imidazolinyl, pyrrolidino, phenothiazinyl, phenoxazinyl, phenazinyl and acridinyl, wherein said substituents are each independently C₁-C₁₂ alkyl, C₁-C₁₂ alkoxy, phenyl or halogen;

(e) a 4-substituted phenyl, the substituent being a dicarboxylic acid residue or derivative thereof, a diamine residue or derivative thereof, an amino alcohol residue or derivative thereof, a polyol residue or derivative thereof, —(CH₂)—, —(CH₂)ₑ— or —└O—(CH₂)ₑ┘_{f}—, wherein e represents an integer ranging from 2 to 6 and f represents an integer ranging from 1 to 50, and wherein the substituent is connected to an aryl group of another photochromic material;

(f) a group represented by:

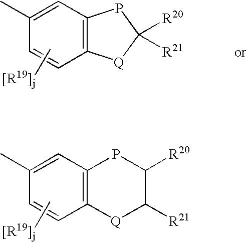

wherein P represents a group, such as, —CH₂— or oxygen; Q represents a group, such as, oxygen or substituted nitrogen, provided that when Q represents substituted nitrogen, P represents —CH₂—, the substituted nitrogen substituents being hydrogen, C₁-C₁₂ alkyl or C₁-C₁₂ acyl; each R¹⁹ independently represents a group, such as, C₁-C₁₂ alkyl, C₁-C₁₂ alkoxy, hydroxy or halogen; R²⁰ and R²¹ each independently represent a group, such as, hydrogen or C₁-C₁₂ alkyl; and j represents an integer ranging from 0 to 2; or

(g) a group represented by:

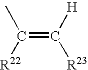

wherein R²² represents a group, such as, hydrogen or C₁-C₁₂ alkyl, and R²³ represents a group, such as, an unsubstituted, mono- or di-substituted naphthyl, phenyl, furanyl or thienyl, wherein said naphthyl, phenyl, furanyl and thienyl substituents are each independently C₁-C₁₂ alkyl, C₁-C₁₂ alkoxy or halogen.

Alternatively, B and B′ may represent groups that together form a fluoren-9-ylidene or mono- or di-substituted fluoren-9-ylidene, each of said fluoren-9-ylidene substituents independently being C₁-C₁₂ alkyl, C₁-C₁₂ alkoxy or halogen.

Further, in structure (iv), R⁴, R⁸, R⁹, and R¹² may each independently represent a group, such as:

(a) hydrogen, C₁-C₆ alkyl, chloro, fluoro, bromo, C₃-C₇ cycloalkyl, a unsubstituted, mono- or di-substituted phenyl, wherein said phenyl substituents are each independently C₁-C₆ alkyl or C₁-C₆ alkoxy;

(b) —OR⁴⁰ or —OC(═O)R⁴⁰, wherein R⁴⁰ represents a group, such as, hydrogen, amine, alkylene glycol, polyalkylene glycol (e.g., as substituent having the general structure —[O—(CₜH₂ₜ)]ᵤ—OR″, wherein t and u are each independently integers ranging from 1 to 10, R″ represents a group, such as, hydrogen, alkyl, a reactive substituent or a second photochromic material, non-limiting examples of which may be found in U.S. Pat. No. 6,113,814 at col. 3, lines 30-64, which disclosure is hereby specifically incorporated by reference herein), C₁-C₆ alkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl, (C₁-C₆)alkoxy(C₁-C₆)alkyl, C₃-C₇ cycloalkyl, mono(C₁-C₄)alkyl substituted C₃-C₇ cycloalkyl or an unsubstituted, mono- or di-substituted phenyl, wherein said phenyl substituents are each independently C₁-C₆ alkyl or C₁-C₆ alkoxy;

(c) a reactive substituent or a compatiblizing substituent (such as those previously discussed);

(d) a 4-substituted phenyl, the substituent being a dicarboxylic acid residue or derivative thereof, a diamine residue or derivative thereof, an amino alcohol residue or derivative thereof, a polyol residue or derivative thereof, —(CH₂)—, —(CH₂)ₑ— or —[O—(CH₂)ₑ]_{f}—, wherein e represents an integer ranging from 2 to 6 and f represents an integer ranging from 1 to 50, and wherein the substituent is connected to an aryl group of another photochromic material (e.g., an aryl group of another indeno-fused naphthopyran);

(e) —N(R⁴¹)R⁴², wherein R⁴¹ and R⁴² independently represent a group, such as, hydrogen, C₁-C₈ alkyl, phenyl, naphthyl, furanyl, benzofuran-2-yl, benzofuran-3-yl, thienyl, benzothien-2-yl, benzothien-3-yl, dibenzofuranyl, dibenzothienyl, benzopyridyl, fluorenyl, C₁-C₈ alkylaryl, C₃-C₈ cycloalkyl, C₄-C₁₆ bicycloalkyl, C₅-C₂₀ tricycloalkyl or C₁-C₂₀ alkoxy(C₁-C₆)alkyl, or R⁴¹ and R⁴² may represent groups that come together with the nitrogen atom to form a C₃-C₂₀ hetero-bicycloalkyl ring or a C₄-C₂₀ hetero-tricycloalkyl ring;

(f) a nitrogen containing ring represented by:

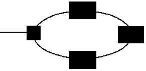

wherein each —V— is independently chosen for each occurrence to represent a group, such as, —CH₂—, —CH(R⁴³)—, —C(R⁴³)₂—, —CH(aryl)-, —C(aryl)₂— and —C(R⁴³)(aryl)-, wherein each R⁴³ independently represents a group, such as, C₁-C₆ alkyl, and each aryl independently represents a group, such as, phenyl or naphthyl; —W— represents a group, such as, —V—, —O—, —S—, —S(O)—, —SO₂—, —NH—, —N(R⁴³)— or —N(aryl)-; s represents an integer ranging from 1 to 3; and r represents an integer ranging from 0 to 3, provided that if r is 0, then —W— is the same as —V—;

(g) a group represented by:

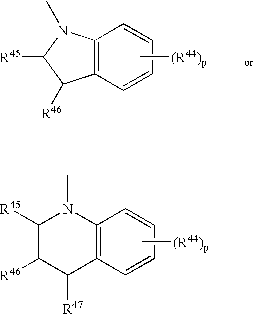

wherein each R⁴⁴ independently represents a group, such as, C₁-C₆ alkyl, C₁-C₆ alkoxy, fluoro or chloro; R⁴⁵, R⁴⁶ and R⁴⁷ each independently represent a group, such as, hydrogen, C₁-C₆ alkyl, phenyl or naphthyl, or R⁴⁵ and R⁴⁶ represent groups that together form a ring of 5 to 8 carbon atoms; and p represents an integer ranging from 0 to 3; or

(h) a substituted or unsubstituted C₄-C₁₈ spirobicyclic amine or a substituted or unsubstituted C₄-C₁₈ spirotricyclic amine, wherein the substituents of the C₄-C₁₈ spirobicyclic amine or the C₄-C₁₈ spirotricyclic amine are each independently aryl, C₁-C₆ alkyl, C₁-C₆ alkoxy or phenyl(C₁-C₆)alkyl.

Non-limiting examples of groups that R⁷ and R¹⁰ (shown above in structure (iv)) may each independently represent include:

(a) the groups discussed above with respect to R⁵, R⁸, R⁹ and R¹², (i.e., any group from which R⁵, R⁸, R⁹ and R¹² may be selected); or

(b) a metallocenyl group (discussed above).

Non-limiting examples of groups that R⁶ and R¹¹ in structure (iv) may each independently represent include:

(a) the groups discussed above with respect to R⁷ and R¹⁰, (i.e., any of the groups discussed above from which any of R⁵, R⁸, R⁹ and R¹² may be selected or a metallocenyl group);

(b) perfluoroalkyl or perfluoroalkoxy;

(c) —C(═O)R⁴⁸ or —SO₂R⁴⁸, wherein each R⁴⁸ independently represents a group, such as, hydrogen, C₁-C₆ alkyl, —OR⁴⁹ or —NR⁵⁰R⁵¹, wherein R⁴⁹, R⁵⁰ and R⁵¹ each independently represents a group, such as, hydrogen, C₁-C₆ alkyl, C₅-C₇ cycloalkyl, alkylene glycol, polyalkylene glycol (e.g., as substituent having the general structure —[O—(CₜH₂ₜ)]ᵤ—OR″, as discussed above) or an unsubstituted, mono- or di-substituted phenyl, wherein said phenyl substituents are each independently C₁-C₆ alkyl or C₁-C₆ alkoxy;

(d) —C(═C(R⁵²)₂)R⁵³, wherein each R⁵² independently represents a group, such as, —C(═O)R⁴⁸, —OR⁴⁹, —OC(═O)R⁴⁹, —NR⁵⁰R⁵¹, hydrogen, halogen, cyano, C₁-C₆ alkyl, C₅-C₇ cycloalkyl, alkylene glycol, polyalkylene glycol (e.g., as substituent having the general structure —[O—(CₜH₂ₜ)]ᵤ—OR″, as discussed above) or an unsubstituted, mono- or di-substituted phenyl, wherein said phenyl substituents are each independently C₁-C₆ alkyl or C₁-C₆ alkoxy; and R⁵³ represents a group, such as, hydrogen, C₁-C₆ alkyl, C₅-C₇ cycloalkyl, alkylene glycol, polyalkylene glycol (as described above) or an unsubstituted, mono- or di-substituted phenyl, wherein said phenyl substituents are each independently C₁-C₆ alkyl or C₁-C₆ alkoxy; or

(e) —C≡CR⁵⁴ or —C≡N, wherein R⁵⁴ represents a group, such as, —C(═O)R⁴⁸ (wherein R⁴⁸ represent groups, such as, those discussed above), hydrogen, C₁-C₆ alkyl, C₅-C₇ cycloalkyl or an unsubstituted, mono- or di-substituted phenyl, wherein said phenyl substituents are each independently C₁-C₆ alkyl or C₁-C₆ alkoxy.

Alternatively, according to various non-limiting embodiments disclosed herein wherein the photochromic material may be represented by structure (iv) above, adjacent groups represented by R⁶ and R⁷ and/or adjacent groups represented by R¹⁰ and R¹¹ may together form a group represented by:

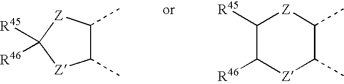

wherein Z and Z′ each independently represents a group, such as, oxygen or the group —NR⁴¹—, wherein R⁴¹, R⁴⁵ and R⁴⁶ may represent groups set forth above; or adjacent groups (e.g., R⁶ and R⁷ and/or R¹⁰ and R¹¹) may together form an aromatic or heteroaromatic fused group, said fused group being benzo, indeno, dihydronaphthalene, indole, benzofuran, benzopyran or thianaphthene. For example, according to one non-limiting embodiment, R⁶ and R⁷ may come together to form a five- or six-membered dioxo ring (i.e., Z and Z′ are both oxygen) wherein R⁴⁵ and R⁴⁶ each independently represents a group, such as, hydrogen, C₁-C₆ alkyl, phenyl or naphthyl, or R⁴⁵ and R⁴⁶ may represent groups that together form a ring of 5 to 8 carbon atoms. According to one specific non-limiting embodiment, R⁶ and R⁷ come together to form a five- or six-membered dioxo ring wherein R⁴⁵ and R⁴⁶ are each hydrogen or C₁-C₆ alkyl.

Non-limiting examples of photochromic materials according to various non-limiting embodiments disclosed herein, wherein the photochromic material is represented by structure (iv) above and comprises a haloalkyl group bonded at the 13-position that is a perhalogenated group, the perhalogenated group being at least one of a perhalo(C₁-C₁₀)alkyl, a perhalo(C₂-C₁₀)alkenyl, a perhalo(C₃-C₁₀)alkynyl, a perhalo(C₁-C₁₀)alkoxy or a perhalo(C₃-C₁₀)cycloalkyl; or a group represented by —O(CH₂)ₐ(CX₂)_{b}CT₃, wherein T represents a halogen, X represents hydrogen or halogen, a represents an integer ranging from 1 to 10, and b represents an integer ranging from 1 to 10 include: 3,3-diphenyl-13-hydroxy-13-trifluoromethyl-3H,13H-indeno[2′,3′:3,4]naphtho[1,2-b]pyran; 3,3-diphenyl-13-trimethylsiloxy-13-trifluoromethyl-3H,13H-indeno[2′,3′:3,4]naphtho[1,2-b]pyran; 3,3-di(4-methoxyphenyl)-6,11-dimethyl-13-trimethylsiloxy-13-trifluoromethyl-3H,13H-indeno[2′,3′:3,4]naphtho[1,2-b]pyran; 3,3-di(4-methoxyphenyl)-6,11,13-trimethyl-13-(2,2,2-trifluoroethoxy)-3H,13H-indeno[2′,3′:3,4]naphtho [1,2-b]pyran; 3,3-di(4-methoxyphenyl)-6-methoxy-7-morpholino-13-ethyl-13-(2,2,2-trifluoroethoxy)-3H,13H-indeno[2′,3′:3,4]naphtho[1,2-b]pyran; 3-(4-fluorophenyl)-3-(4-methoxyphenyl)-6-methoxy-7-morpholino-13-butyl-13-(2,2,2-trifluoroethoxy)-3H,13H-indeno[2′,3′:3,4]naphtho[1,2-b]pyran; 3-(4-fluorophenyl)-3-(4-methoxyphenyl)-6-methoxy-7-morpholino-13-ethyl-13-(2,2,2-trifluoroethoxy)-3H,13H-indeno[2′,3′:3,4]naphtho[1,2-b]pyran; 3-(4-fluorophenyl)-3-(4-methoxyphenyl)-6-methoxy-7-morpholino-13-butyl-13-(1H,1H,2H,2H-perfluorododecanoxy)-3H,13H-indeno└2′,3′:3,4┘naphtho└1,2-b┘pyran; 3-(4-fluorophenyl)-3-(4-methoxyphenyl)-6-methoxy-7-morpholino-13-butyl-13-(3-perfluorobutylpropoxy)-3H,13H-indeno┌2′,3′:3,4┐naphtho┌1,2-b┐pyran; 3,3-di(4-methoxyphenyl)-6-methoxy-7-morpholino-13-ethyl-13-(2,2,3,3,3-pentafluoropropoxy)-3H,13H-indeno[2′,3′:3,4]naphtho[1,2-b]pyran; 3,3-di(4-methoxyphenyl)-6-methoxy-7-morpholino-13-ethyl-13-(2,2,3,3,4,4,4-heptafluorobutoxy)-3H,13H-indeno[2′,3′:3,4]naphtho[1,2-b]pyran; or mixtures thereof.

As previously discussed, photochromic materials are materials that are adapted to display photochromic properties, that is, they are adapted to have an absorption spectrum for at least visible radiation that varies in response to at least actinic radiation. Generally speaking, a photochromic material will have a first absorption spectrum associated with its ground-state form and a second absorption spectrum associated with its activated-state form, that is, the form of the photochromic material on exposure to actinic radiation. Further, as previously discussed, for many optical applications, such as, for example, ophthalmic eyewear applications, it may be desirable that the ground-state form of the photochromic material have as high transmittance for visible radiation as possible so that the eyewear incorporating the photochromic material has good clarity, that is the eyewear has essentially no visible color (i.e., is optically clear) when not exposed to actinic radiation.

According to various non-limiting embodiments disclosed herein, the photochromic material comprising the indeno[2′,3′:3,4]naphtho[1,2-b]pyran and the haloalkyl group bonded at the 13-position of the indeno┌2′,3′:3,4┐naphtho┌1,2-b┐pyran may have an ground-state form absorption spectrum for electromagnetic radiation (that is an absorption spectrum of the photochromic material when not exposed to actinic radiation) that is hypsochromically shifted such that the ground-state form absorption spectrum has no absorption maxima in the visible region of the electromagnetic spectrum. As used herein, the term “hypsochromically shifted” means having an absorption spectrum for electromagnetic radiation that is shifted to shorter wavelengths (i.e., higher frequencies).

More specifically, according to various non-limiting embodiments of the present disclosure, the photochromic material comprising the indeno┌2′,3′:3,4┐naphtho┌1,2-b┐pyran and the haloalkyl group bonded at the 13-position of the indeno[2′,3′:3,4]naphtho[1,2-b]pyran may have a ground-state form absorption spectrum for electromagnetic radiation that is hypsochromically shifted as compared to the ground state-form absorption spectrum of a photochromic material comprising a comparable indeno[2′,3′:3,4]naphtho[1,2-b]pyran without the haloalkyl at the 13-position thereof. That is, as compared to an indeno└2′,3′:3,4┘naphtho└1,2-b┘pyran with a comparable structure except for replacing the haloalkyl or haloalkoxy group bonded at the 13-position with a hydrogen, methyl group or other equivalent non-halogenated group, the photochromic materials according to various non-limiting embodiments disclosed herein may have a ground-state form absorption spectrum for visible radiation that is shifted toward shorter wavelengths.

Further, because the photochromic materials according to various non-limiting embodiments disclosed herein may have a ground-state form absorption spectrum for electromagnetic radiation that is hypsochromically shifted as compared to comparable photochromic materials without the 13-position haloalkyl group, the photochromic materials according to various non-limiting embodiments disclosed herein may absorb less visible radiation in their ground-state forms as compared to comparable photochromic materials. Consequently, photochromic compositions and articles that comprise the photochromic materials according to various non-limiting embodiments disclosed herein may have a clear or colorless state having greater clarity, that is a greater transmittance for visible radiation, as compared to photochromic compositions and articles comprising a comparable indeno[2′,3′:3,4]naphtho[1,2-b]pyran without the haloalkyl group at the 13-position thereof.

According to one specific non-limiting embodiment of the present disclosure, the photochromic material comprising the indeno[2′,3′:3,4]naphtho[1,2-b]pyran and a haloalkyl group bonded at the 13-position of the indeno[2′,3′:3,4]naphtho[1,2-b]pyran may have a ground-state form absorption spectrum for electromagnetic radiation having no absorption maxima in the visible region of the electromagnetic spectrum at wavelengths greater than 410 nm. Since the photochromic materials according to these non-limiting embodiments display no significant absorbance of visible radiation in their ground-state form, the photochromic materials, and consequently photochromic compositions and/or articles comprising the photochromic materials, may have greater transmittance of visible light in their optically clear, ground-state form as compared to comparable photochromic materials. Generally speaking, as the transmittance of visible light through an ophthalmic lens increases, the visual acuity and comfort provided by the lens to the wearer also increases.

Additionally, as previously discussed, for certain applications, such as, but not limited to ophthalmic eyewear applications, it may be advantageous to have a photochromic material that transitions quickly from its optically clear state to its colored state and/or from its colored state to its optically clear state, that is, a photochromic material having “fast” activation and/or fade rates. Throughout the present disclosure, the term “fade rate” represents a kinetic rate value that may be expressed by the T_{1/2} value of the photochromic material. “Fade rate” is a measurement of the rate at which the photochromic material transforms from the colored, activated-state form to the optically clear, ground-state form. The fade rate of a photochromic material may be measured, for example, by activating a photochromic material to saturation under controlled conditions in a given matrix, measuring its activated steady state absorbance (i.e., its saturated optical density) and then determining the length of time it takes for the absorbance of the photochromic material to decrease to one-half the activated steady state absorbance value. As measured in this fashion, the fade rate may be designated by T_{1/2}, with units of seconds. Thus, when the fade rate is said to be fast or faster, the photochromic material changes from the colored state to the optically clear state at a faster rate. The faster fade rate may be indicated, for example, by a lower T_{1/2} value for the photochromic material. That is, as the fade rate becomes faster, the length of time for the absorbance to decrease to one-half the initial activated absorbance value will become shorter. More detailed measurement procedures for determining the T_{1/2} values for the photochromic materials disclosed herein are set forth in the Examples below.

It will be appreciated by those skilled in the art that the fade rate of the photochromic material may be dependent on the media into which the photochromic material is incorporated. As used herein in relation to a photochromic material in a media, the term “incorporated” means physically and/or chemically combined with. In the present disclosure, all photochromic performance data, including fade rate values as denoted by T_{1/2} values and hypsochromic shift values, disclosed herein are measured using a standard protocol involving incorporation of the photochromic material into a polymer test chip comprising a methacrylate polymer, unless specifically noted otherwise. Photochromic performance testing and the standard protocol for formation of the polymer test chip, which incorporates the photochromic material according to various non-limiting embodiments disclosed herein, are provided in greater detail in the Examples section below. One skilled in the art will recognize that although exact values for fade rates and other photochromic performance data, such as, for example, hypsochromic shift data, may vary depending upon the medium into which the photochromic material is incorporated, the photochromic performance data presented herein may be illustrative of relative rates and shifts that may be expected for the photochromic material when incorporated into other media.

According to various non-limiting embodiments disclosed herein, the photochromic materials comprising the indeno[2′,3′:3,4]naphtho[1,2-b]pyran and a haloalkyl group bonded at the 13-position of the indeno-fused naphthopyran may have a faster fade rate as compared to a comparable indeno-fused naphthopyran with hydrogen or methyl groups bonded at the 13-position thereof.

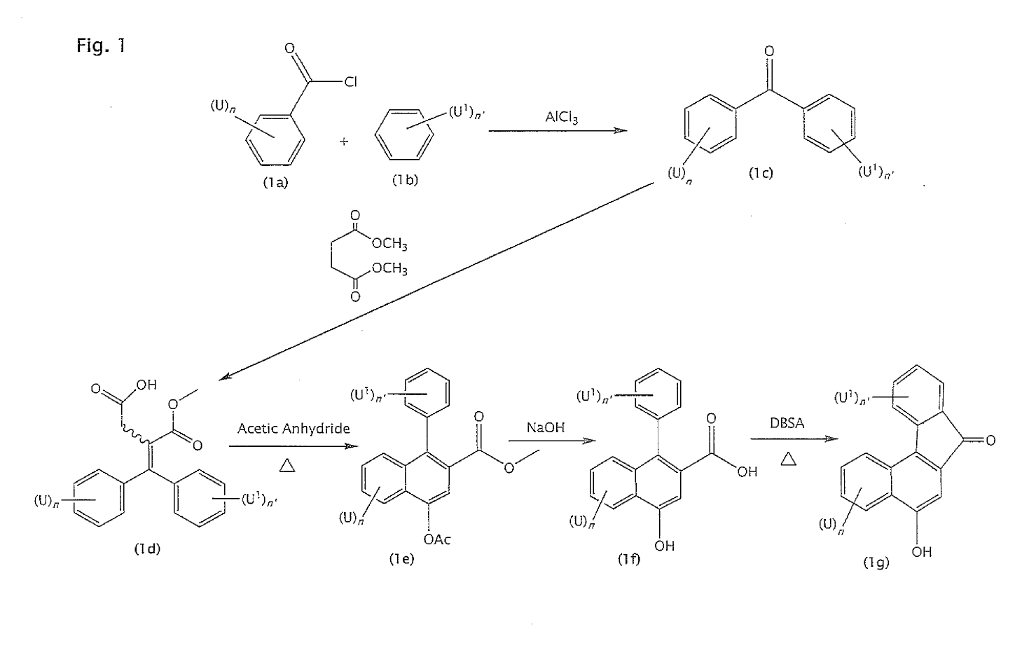

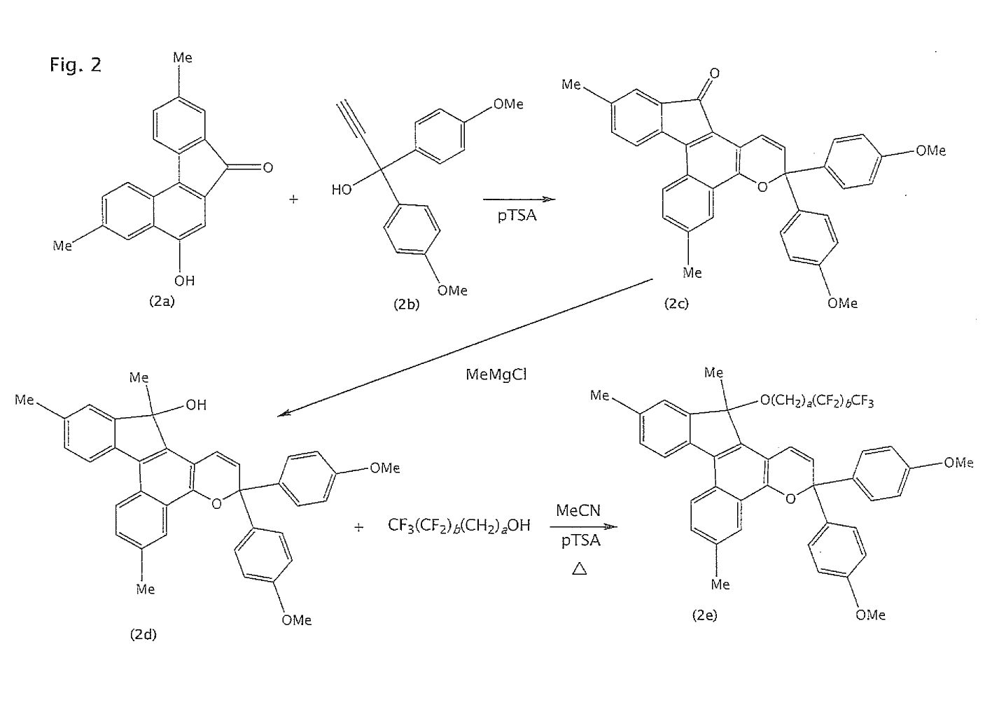

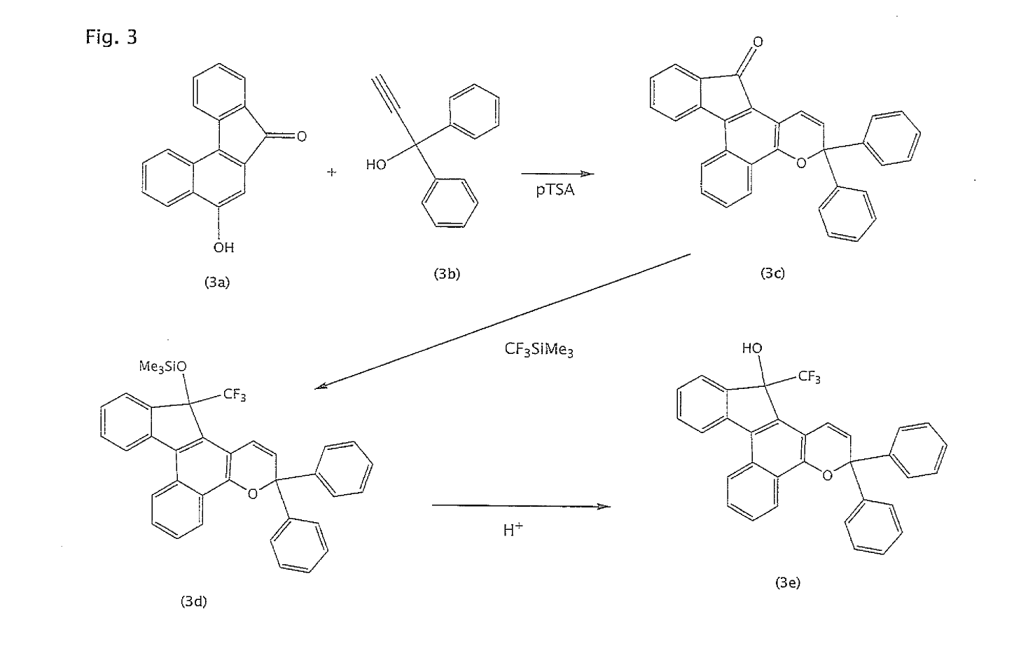

Methods of making photochromic materials according to various non-limiting embodiments will now be described with reference to FIGS. 1-3. FIG. 1 depicts a generalized reaction scheme for making substituted 7H-benzo[C]fluorenone compounds that may be further reacted, for example as shown in FIGS. 2 and 3, to form photochromic materials comprising a haloalkyl group bonded at the 13-position according to various non-limiting embodiments disclosed herein. It should be appreciated that these reaction schemes are presented for illustration only and are not intended to be limiting herein. Additional examples of methods of making photochromic materials according to various non-limiting embodiments disclosed herein are set forth in the Examples.

Referring now to FIG. 1, a solution of a benzoyl chloride, represented by structure (1a) in FIG. 1, which may have a substituent U (where n is an integer ranging from 0 to 4), and benzene, represented by structure (1b) in FIG. 1, which may have a substituent U¹ (where n′ is an integer ranging from 0 to 4), in methylene chloride are added to a reaction flask. Non-limiting examples of groups that U may represent include those groups discussed above with respect to R⁵-R⁸. Non-limiting examples of groups that U¹ may represent include those groups discussed above with respect to R⁹-R¹². Anhydrous aluminum chloride may be used to catalyze the Friedel-Crafts acylation to give a substituted benzophenone represented by structure (1c) in FIG. 1. This material may then be reacted with dimethyl succinate (in a Stobbe reaction) to produce a mixture of half-acid, half-ester, which mixture is generally represented by structure (1d) in FIG. 1. Thereafter, the mixture of half-acid, half-ester (1d) may be reacted in acetic anhydride and toluene at an elevated temperature to produce, after recrystallization, a mixture of two substituted naphthalene compounds (when U is not the same as U¹), one of which is generally represented by structure (1e) in FIG. 1. The two substituted naphthalene compounds are then hydrolyzed in a sodium hydroxide solution to form a mixture of two hydrolyzed compounds, one of which is represented by structure (1f) in FIG. 1. The hydrolyzed substituted naphthalene compounds are then cyclized with dodecylbenzene sulfonic acid to afford a mixture of two substituted 7H-benzo┌C┐fluorenone compounds, one of which is generally represented by structure (1g) in FIG. 1. The mixtures may be separated by conventional means at any convenient point during the synthesis. Other non-limiting method of forming hydroxy-substituted 7H-benzo[C]fluorenone compounds that may be useful in forming photochromic material according to various non-limiting embodiments disclosed herein are described in U.S. Pat. No. 6,296,785 at col. 10, line 52 to col. 13, line 22, and col. 19, line 16 to col. 21, line 28 (“Reaction J”), which disclosure is hereby specifically incorporated by reference herein. Non-limiting methods of forming 7H-benzo┌C┐fluoren-5-ol compounds, which may be further reacted with a substituted 2-propyn-1-ol to form photochromic materials according to various non-limiting embodiments disclosed herein are described in U.S. Pat. No. 6,296,785 at col. 16, lines 1 to 15 (“Reaction F”), and col. 21, line 29 to col. 23, line 14 (“Reaction K”), which disclosure is hereby specifically incorporated by reference herein.

Referring now to FIG. 2, a substituted 7II-benzo[C]fluorenone, represented by structure (2a) in FIG. 2, may be formed as described in FIG. 1, wherein U and U¹ each represent methyl, and reacted with a substituted 2-propyn-1-ol, such as, the substituted 2-propyn-1-ol represented by structure (2b) in FIG. 2 in the presence of an acid (e.g., p-toluenesulfonic acid as shown in FIG. 2) to form the indeno[2′,3′:3,4]naphtho[1,2-b]pyran represented by structure (2c) in FIG. 2. The indeno[2′,3′:3,4]naphtho[1,2-b]pyran represented by structure (2c) can be further reacted with a carbanion equivalent, such as R″MgX (e.g., methyl magnesium chloride as shown in FIG. 2), to produce a compound represented by structure (2d) in FIG. 2. Further reaction of the compound represented by structure (2d) with a haloalkanol (e.g., CF₃(CF₂)_{b}(CH₂)ₐOH as shown in FIG. 2) in the presence of an acid (e.g., p-toluenesulfonic acid as shown in FIG. 2) and a solvent (e.g., acetonitrile as shown in FIG. 2) results in the formation of an indeno[2′,3′:3,4]naphtho[1,2-b]pyran comprising a haloalkyloxy group, represented by —O(CH₂)ₐ(CX₂)_{b}CT₃, wherein X and T are fluorine, bonded at the 13-position thereof, according to one non-limiting embodiment of the present invention and represented by structure (2e) in FIG. 2.

Referring now to FIG. 3, a substituted 7H-benzo[C]fluorenone, represented by structure (3a) in FIG. 3, may be formed as described in FIG. 1 for structure (1g), wherein U and U¹ each represent hydrogen, and reacted with a diaryl substituted 2-propyn-1-ol, such as, 1,1-diphenyl-2-propyn-1-ol represented by structure (3b) in FIG. 3 in the presence of an acid (e.g., p-toluenesulfonic acid as shown in FIG. 3) to form the indeno[2′,3′:3,4]naphtho[1,2-b]pyran represented by structure (3c) in FIG. 3. The indeno[2′,3′:3,4]naphtho[1,2-b]pyran represented by structure (3c) can be further reacted with a perfluoroalkyltrialkylsilane (e.g., CF₃Si(CH₃)₃ as shown in FIG. 3) to form an indeno[2′,3′:3,4]naphtho[1,2-b]pyran represented by structure (3d) according to one non-limiting embodiment disclosed herein, wherein the indeno[2′,3′:3,4]naphtho[1,2-b]pyran comprises a haloalkyl group represented by —CF₃ and a trimethylsiloxy group, each of which is bonded at the 13-position thereof.

Further, the compound represented by structure (3d) can be reacted in the presence of a catalytic amount of an acid to form the indeno└2′,3′:3,4┘naphtho└1,2-b┘pyran represented by structure (3e) according to one non-limiting embodiment of the present disclosure, wherein the indeno┌2′,3′:3,4┐naphtho┌1,2-b┐pyran comprises a haloalkyl group represented by —CF₃ and a hydroxyl group, each of which is bonded at the 13-position thereof.

One skilled in the art will recognize that various changes or modifications may be made to the synthesis procedures described above and illustrated in FIGS. 1-3 without deviating from the scope and nature of the invention as described herein and set forth in the claims. As indicated above, these reaction schemes are presented for illustration only and are not intended to be limiting herein.

In one non-limiting embodiment, a photochromic material, such as 3,3-di(4-methoxyphenyl)-6-methoxy-7-morpholino-13-ethyl-13-hydroxy-3H,13H-indeno┌2′,3′:3,4┐naphtho[1,2-b]pyran (the starting material for Examples 4, 8, and 9 and Comparative Example CE4, as described in the Examples section herein), can be prepared by reacting 2-morpholino-3-methoxy-5,7-dihydroxy-7-ethyl-7H-benzo[C]fluorene, which can be prepared by following Step 2 of Example 9 of U.S. Pat. No. 6,296,785 using the appropriately substituted starting material (which example is hereby incorporated herein by reference) with 1,1-bis(4-methoxyphenyl)-2-propyn-1-ol, which can be prepared by following the method of Step 1 of Example 1 of U.S. Pat. No. 5,458,814 (which example is hereby incorporated herein by reference) using procedures known to those skilled in the art.

In another non-limiting embodiment, the intermediate 2,3-dimethoxy-5-acetoxy-7H-benzo┌C┐fluoren-7-one which is used herein for the preparation of Examples 5 and 6 and Comparative Examples CE2 and CE5 (as described in the Examples section herein) can be prepared by following Step 2 of Example 9 of U.S. Pat. No. 6,296,785 (which example is hereby incorporated herein by reference) using the appropriately substituted starting material and procedures known to those skilled in the art.

In a further non-limiting embodiment, the photochromic material 3,3-di(4-methoxyphenyl)-6,11-dimethyl-13-oxo-3H,13H-indeno[2′,3′:3,4]naphtho[1,2-b]pyran which is used herein for the preparation of Example 7 (as described in the Examples section herein) can be prepared following the procedure of Example 5 of U.S. Pat. No. 5,645,767 (which example is hereby incorporated herein by reference) using procedures known to those skilled in the art.

In a still further non-limiting embodiment, the photochromic material 3,3-diphenyl-13-oxo-3H,13H-indeno[2′,3′:3,4]naphtho[1,2-b]pyran which is used herein for the preparation of Example 1 (as described in the Examples section herein) can be prepared following the procedure of Steps 1 through 6 of Example 1 of U.S. Pat. No. 5,645,767 (which example is hereby incorporated herein by reference) except that in Step 6, 1,1-diphenyl-2-propyn-1-ol was used in place of 1,1-di(4-methoxyphenyl)-2-propyn-1-ol, using procedures known to those skilled in the art.

As discussed above, the photochromic materials according to various non-limiting embodiments disclosed herein may be incorporated into at least a portion of an organic material, such as, a polymeric, oligomeric or monomeric material to form a photochromic composition, which photochromic composition may be used, for example and without limitation, to form photochromic articles, such as, optical elements, and coating compositions that may be applied to various substrates. As used herein, the terms “polymer” and “polymeric material” refer to homopolymers and copolymers (e.g., random copolymers, block copolymers, and alternating copolymers), as well as blends and other combinations thereof. As used herein, the terms “oligomer” and “oligomeric material” refer to a combination of two or more monomer units that are capable of reacting with additional monomer unit(s). As used herein, the term “incorporated into” means physically and/or chemically combined with. For example, the photochromic materials according to various non-limiting embodiments disclosed herein may be physically combined with at least a portion of an organic material, for example and without limitation, by mixing or imbibing the photochromic material into the organic material; and/or chemically combined with at least a portion of an organic material, for example and without limitation, by copolymerization or otherwise bonding the photochromic material to the organic material.

Further, it is contemplated that the photochromic materials according to various non-limiting embodiments disclosed herein may each be used alone in the photochromic compositions and articles disclosed herein, or may be used in combination with other photochromic materials according to various non-limiting embodiments disclosed herein, or in combination with an appropriate complementary conventional photochromic material. For example, the photochromic materials according to various non-limiting embodiments disclosed herein may be used in conjunction with conventional photochromic materials having activated-state form absorption maxima within the range of 300 to 1000 nanometers, for example, from 400 to 800 nanometers. Further, the photochromic materials according to various non-limiting embodiments disclosed herein may be used in conjunction with a complementary conventional polymerizable or a compatiblized photochromic material, such as, for example, those disclosed in U.S. Pat. Nos. 6,113,814 (at col. 2, line 39 to col. 8, line 41), and U.S. Pat. No. 6,555,028 (at col. 2, line 65 to col. 12, line 56), which disclosures are hereby specifically incorporated by reference herein.

As discussed above, according to various non-limiting embodiments disclosed herein, the photochromic compositions (as well as the photochromic articles discussed herein) may contain a mixture of photochromic materials. For example, although not limiting herein, mixtures of photochromic materials may be used to attain certain activated colors, such as, a near neutral gray or near neutral brown. For example, U.S. Pat. No. 5,645,767, col. 12, line 66 to col. 13, line 19, describes the parameters that define neutral gray and brown colors and which disclosure is specifically incorporated by reference herein.

Various non-limiting embodiments disclosed herein provide a photochromic composition comprising an organic material, the organic material being at least one of polymeric material, an oligomeric material and a monomeric material, and a photochromic material according to any of the non-limiting embodiments of set forth above incorporated into at least a portion of the organic material. According to various non-limiting embodiments disclosed herein, the photochromic material may be incorporated into a portion of the organic material by blending and/or bonding the photochromic material with the organic material or a precursor thereof. As used herein with reference to the incorporation of photochromic materials into an organic material, the terms “blending” and “blended” mean that the photochromic material is intermixed or intermingled with the at least a portion of the organic material, but not bonded to the organic material. Further, as used herein with reference to the incorporation of photochromic materials into an organic material, the terms “bonding” or “bonded” mean that the photochromic material is linked to a portion of the organic material or a precursor thereof. For example, although not limiting herein, the photochromic material may be linked to the organic material through a reactive substituent, such as, those as discussed above.

According to one non-limiting embodiment wherein the organic material is a polymeric material, the photochromic material may be incorporated into at least a portion of the polymeric material or at least a portion of a monomeric material or oligomeric material from which the polymeric material is formed. For example, photochromic materials according to various non-limiting embodiments disclosed herein that have a reactive substituent may be bonded to an organic material, such as, a monomer, oligomer or polymer, having a group with which a reactive moiety of the reactive substituent may be reacted, or the reactive moiety may be reacted as a co-monomer in the polymerization reaction from which the organic material is formed, for example, in a co-polymerization process.

As discussed above, the photochromic compositions according to various non-limiting embodiments disclosed herein may comprise an organic material chosen from a polymeric material, an oligomeric material and/or a monomeric material. Examples of polymeric materials that may be used in conjunction with various non-limiting embodiments disclosed herein include, without limitation: polymers of bis(allyl carbonate) monomers; diethylene glycol dimethacrylate monomers; diisopropenyl benzene monomers; ethoxylated bisphenol A dimethacrylate monomers; ethylene glycol bismethacrylate monomers; poly(ethylene glycol) bismethacrylate monomers; ethoxylated phenol bismethacrylate monomers; alkoxylated polyhydric alcohol acrylate monomers, such as, ethoxylated trimethylol propane triacrylate monomers; urethane acrylate monomers; vinylbenzene monomers; and styrene. Other non-limiting examples of suitable polymeric materials include polymers of polyfunctional, e.g., mono-, di- or multi-functional, acrylate and/or methacrylate monomers; poly(C₁-C₁₂ alkyl methacrylates), such as, poly(methyl methacrylate); poly(oxyalkylene)-dimethacrylate; poly(alkoxylated phenol methacrylates); cellulose acetate; cellulose triacetate; cellulose acetate propionate; cellulose acetate butyrate; poly(vinyl acetate); poly(vinyl alcohol); poly(vinyl chloride); poly(vinylidene chloride); polyurethanes; polythiourethanes; thermoplastic polycarbonates; polyesters; poly(ethylene terephthalate); polystyrene; poly(α-methylstyrene); copolymers of styrene and methyl methacrylate; copolymers of styrene and acrylonitrile; polyvinylbutyral; and polymers of diallylidene pentaerythritol, particularly copolymers with polyol (allyl carbonate) monomers, e.g., diethylene glycol bis(allyl carbonate), and acrylate monomers, e.g., ethyl acrylate and butyl acrylate. Also contemplated are copolymers of the aforementioned monomers, combinations, and blends of the aforementioned polymers and copolymers with other polymers, e.g., to form interpenetrating network products.

Further, according to various non-limiting embodiments wherein transparency of the photochromic composition is desired, the organic material may be a transparent polymeric material. For example, according to various non-limiting embodiments, the polymeric material may be an optically clear polymeric material prepared from a thermoplastic polycarbonate resin, such as, the resin derived from bisphenol A and phosgene, which is sold under the trademark, LEXAN®; a polyester, such as, the material sold under the trademark, MYLAR®; a poly(methyl methacrylate), such as, the material sold under the trademark, PLEXIGLAS®; polymerizates of a polyol(allyl carbonate) monomer, especially diethylene glycol bis(allyl carbonate), which monomer is sold under the trademark CR-39®; and polyurea-polyurethane (polyurea urethane) polymers, which are prepared, for example, by the reaction of a polyurethane oligomer and a diamine curing agent, a composition for one such polymer being sold under the trademark TRIVEX® by PPG Industries, Inc. Other non-limiting examples of suitable polymeric materials include polymerizates of copolymers of a polyol (allyl carbonate), e.g., diethylene glycol bis(allyl carbonate), with other copolymerizable monomeric materials, such as, but not limited to: copolymers with vinyl acetate, copolymers with a polyurethane having terminal diacrylate functionality, and copolymers with aliphatic urethanes, the terminal portion of which contain allyl or acrylyl functional groups. Still other suitable polymeric materials include, without limitation, poly(vinyl acetate), polyvinylbutyral, polyurethane, polythiourethanes, polymers chosen from diethylene glycol dimethacrylate monomers, diisopropenyl benzene monomers, ethoxylated bisphenol A dimethacrylate monomers, ethylene glycol bismethacrylate monomers, poly(ethylene glycol) bismethacrylate monomers, ethoxylated phenol bismethacrylate monomers and ethoxylated trimethylol propane triacrylate monomers, cellulose acetate, cellulose propionate, cellulose butyrate, cellulose acetate butyrate, polystyrene and copolymers of styrene with methyl methacrylate, vinyl acetate and acrylonitrile. According to certain non-limiting embodiment, the polymeric materials may be an optical resins sold by PPG Industries, Inc. under the CR-designation, e.g., CR-307, CR-407, and CR-607.

According to one specific non-limiting embodiment, the organic material may be a polymeric material chosen from poly(carbonate), copolymers of ethylene and vinyl acetate; copolymers of ethylene and vinyl alcohol; copolymers of ethylene, vinyl acetate, and vinyl alcohol (such as those that result from the partial saponification of copolymers of ethylene and vinyl acetate); cellulose acetate butyrate; poly(urethane); poly(acrylate); poly(methacrylate); epoxies; aminoplast functional polymers; poly(anhydride); poly(urea urethane); N-alkoxymethyl(meth)acrylamide functional polymers; poly(siloxane); poly(silane); and combinations and mixtures thereof.

Further, it will be appreciated by those skilled in the art that the photochromic compositions according to various non-limiting embodiments disclosed herein may further comprise other additives that aid in the processing and/or performance of the composition or a coating or article derived therefrom. Non-limiting examples of such additives include photoinitiators, thermal initiators, polymerization inhibitors, solvents, light stabilizers (such as, but not limited to, ultraviolet light absorbers and light stabilizers, such as, hindered amine light stabilizers (HALS)), heat stabilizers, mold release agents, rheology control agents, leveling agents (such as, but not limited to, surfactants), free radical scavengers, adhesion promoters (such as, hexanediol diacrylate and coupling agents), and combinations and mixtures thereof.

As previously discussed, the present invention further contemplates photochromic articles, such as, optical elements, made using the photochromic materials and/or the photochromic compositions according to various non-limiting embodiments disclosed herein. As used herein, the term “optical” means pertaining to or associated with light and/or vision. The optical elements according to various non-limiting embodiments disclosed herein may include, without limitation, ophthalmic elements, display elements, windows, mirrors, and liquid crystal cell elements. As used herein, the term “ophthalmic” means pertaining to or associated with the eye and vision. Non-limiting examples of ophthalmic elements include corrective and non-corrective lenses, including single vision or multi-vision lenses, which may be either segmented or non-segmented multi-vision lenses (such as, but not limited to, bifocal lenses, trifocal lenses and progressive lenses), as well as other elements used to correct, protect or enhance (cosmetically or otherwise) vision, including without limitation, contact lenses and other intraocular elements, magnifying lenses, protective lenses, visors, goggles, as well as, lenses for optical instruments (for example, cameras and telescopes). As used herein, the term “display” means the visible or machine-readable representation of information in words, numbers, symbols, designs or drawings. Non-limiting examples of display elements include screens, monitors, and security elements, such as, security marks. As used herein, the term “window” means an aperture adapted to permit the transmission of radiation therethrough. Non-limiting examples of windows include automotive and aircraft transparencies, windshields, filters, shutters, and optical switches. As used herein, the term “mirror” means a surface that specularly reflects a large fraction of incident light. As used herein, the term “liquid crystal cell” refers to a structure containing a liquid crystal material that is capable of being ordered. One non-limiting example of a liquid crystal cell element is a liquid crystal display.

Various non-limiting embodiments disclosed herein provide photochromic articles, such as, optical elements, comprising a substrate and a photochromic material according to any of the non-limiting embodiments discussed above connected to a portion of the substrate. As used herein, the term “connected to” means associated with, either directly or indirectly through another material or structure. Further, as used herein in the context of a coating being “on” a surface or object, the term “on” means that the subject coating is connected to the surface or object such that the subject coating is supported or carried by the surface or object. For example, a coating that is “on” a surface may be applied directly over the surface or it may be applied over one or more other coatings, at least one of which is applied directly over the surface.

According to various non-limiting embodiments disclosed herein wherein the substrate of the photochromic article comprises a polymeric material, the photochromic material may be connected to at least a portion of the substrate by incorporating the photochromic material into at least a portion of the polymeric material of the substrate, or by incorporating the photochromic material into at least a portion of the oligomeric or monomeric material from which the substrate is formed. For example, according to one non-limiting embodiment, the photochromic article may be formed from a photochromic composition, such as, those discussed above, by the cast-in-place method wherein the photochromic material is incorporated into at least a portion of the polymeric material of the substrate by blending and/or bonding the photochromic material with at least a portion of the polymeric material prior to forming the substrate, or by incorporating the photochromic material into at least a portion of the oligomeric or monomeric material from which the polymeric material of the substrate is formed prior to forming the substrate. According to other non-limiting embodiments, the photochromic material may be incorporated into the polymeric material of the substrate by imbibition. Imbibition and the cast-in-place method are discussed below in more detail.

According to still other non-limiting embodiments, the photochromic material may be connected to at least a portion of the substrate of the photochromic article as part of an at least partial coating that is connected to at least a portion of a substrate. As used herein, the term “coating” means a structure comprising one or more complete or partial layers (which may or may not have a uniform composition and/or cross-sectional thickness) derived from flowable compositions. The flowable compositions from which coatings may be formed include, for example, liquid or powder compositions, which may be applied to the substrate using methods, such as, those discussed herein below. According to these non-limiting embodiments, the substrate may be a polymeric substrate or an inorganic substrate (such as, but not limited to, a glass substrate). Examples of monomers and polymers that may be used to form the polymeric substrates according to various non-limiting embodiments disclosed herein include, but are not limited to, those monomers and polymers discussed above that may be useful in forming the photochromic compositions according to various non-limiting embodiments disclosed herein.

According to one non-limiting embodiment disclosed herein, the substrate may be an ophthalmic substrate. As used herein, the term “ophthalmic substrate” refers to lenses, partially formed lenses, and lens blanks. Non-limiting examples of organic materials from which ophthalmic substrates according to various non-limiting embodiments disclosed herein may be formed include, but are not limited to, art-recognized polymers that are useful in forming transparent or optically clear castings for optical applications (such as those previously discussed).

Other non-limiting examples of organic materials suitable for use in forming the substrates according to various non-limiting embodiments disclosed herein include both synthetic and natural organic materials, including without limitation: opaque or translucent polymeric materials, natural and synthetic textiles, and cellulosic materials. Non-limiting examples of inorganic materials suitable for use in forming substrates that may be used in conjunction with various non-limiting embodiments disclosed herein include inorganic oxide-based glasses, minerals, ceramics, and metals. For example, in one non-limiting embodiment the substrate may comprise glass. In other non-limiting embodiments, the substrate may be a ceramic, metal or mineral substrate that has been polished to form a reflective surface. In other non-limiting embodiments, a reflective coating or layer may be deposited or otherwise applied to a surface of an inorganic or an organic substrate to make it reflective or enhance its reflectivity.

According to various non-limiting embodiments disclosed herein, the substrate may comprise a protective coating on at least a portion of its surface. As used herein, the term “protective coating” refers to coatings or films that can prevent wear or abrasion, provide a transition in properties from one coating or film to another, protect against the effects of polymerization reaction chemicals and/or protect against deterioration due to environmental conditions, such as, moisture, heat, ultraviolet light, oxygen, etc. For example, commercially available thermoplastic polycarbonate ophthalmic lens substrates are often sold with an abrasion-resistant coating already applied to their surfaces because these surfaces tend to be readily scratched, abraded or scuffed. An example of one such polycarbonate lens substrate is sold under the trademark GENTEX (by Gentex Optics). Non-limiting examples of abrasion-resistant coatings include, abrasion-resistant coatings comprising silanes, abrasion-resistant coatings comprising radiation-cured acrylate-based thin films, abrasion-resistant coatings based on inorganic materials, such as, silica, titania and/or zirconia, and combinations thereof. For example, according to various non-limiting embodiments the protective coating may comprise a first coating of a radiation-cured acrylate-based thin film and a second coating comprising a silane. Non-limiting examples of commercial protective coatings products include SILVUE® 124 and HI-GARD® coatings, commercially available from SDC Coatings, Inc. and PPG Industries, Inc., respectively.

According to various non-limiting embodiments disclosed herein, the photochromic material according to various non-limiting embodiments of the present invention discussed above may be incorporated into at least a portion of a coating composition prior to application of the coating composition to the substrate, or alternatively, a coating composition may be applied to the substrate, at least partially set, and thereafter the photochromic material may be imbibed into at least a portion of the coating. As used herein with reference to coatings, coating compositions, or components thereof, the terms “set” and “setting” are intended to include processes, such as, but not limited to, curing, polymerizing, cross-linking, cooling, and drying.

Specific non-limiting examples of coating composition into which the photochromic materials according to various non-limiting embodiments disclosed herein may be incorporated include, but are not limited to, those coating compositions known in the art for use in connection with photochromic materials. Non-limiting examples of a coating compositions into which the photochromic materials according to various non-limiting embodiments disclosed herein may be incorporated include the mono-isocyanate containing coating compositions disclosed in U.S. Pat. No. 6,916,537 (“the '537 Patent”) at col. 3, lines 1 to 12, which comprises (in addition to a photochromic material) a reaction product (non-limiting examples which are set forth in the '537 Patent at col. 7, lines 4-37) of a polyol comprising at least one carbonate group (non-limiting examples of which are set forth in the '537 Patent at col. 7, line 38 to col. 8, line 49) and an isocyanate comprising at least one reactive isocyanate group and at least one polymerizable double bond (non-limiting examples of which are set forth in the '537 Patent at col. 8, line 50 to col. 9, line 44), and which optionally comprises an addition copolymerizable monomer (non-limiting examples of which are set forth in the '537 Patent at col. 11, line 47 to col. 20, line 43). The above-referenced disclosure of the '537 Patent is hereby specifically incorporated by reference herein.

Other non-limiting examples of coating compositions into which the photochromic materials according to various non-limiting embodiments disclosed herein may be incorporated include the poly(urea-urethane) compositions disclosed in U.S. Pat. No. 6,531,076, at col. 3, line 4 to col. 10, line 49, which disclosure is hereby specifically incorporated by reference herein. Still other non-limiting examples of coating compositions into which the photochromic materials according to various non-limiting embodiments disclosed herein may be incorporated include the polyurethane compositions disclosed in U.S. Pat. No. 6,187,444, at col. 2, line 52 to col. 12, line 15, which disclosure is hereby specifically incorporated by reference herein.

Yet other non-limiting examples of coating compositions into which the photochromic materials according to various non-limiting embodiments disclosed herein may be incorporated include the poly(meth)acrylic coating compositions described in U.S. Pat. No. 6,602,603, at col. 2, line 60 to col. 7, line 50; the aminoplast resin coating compositions described in U.S. Pat. No. 6,506,488, at col. 2, line 43 to col. 12, line 23 and U.S. Pat. No. 6,432,544, at col. 2, line 32 to col. 14, line 5; the polyanhydride coating compositions described in U.S. Pat. No. 6,436,525, at col. 2, line 15 to col. 11, line 60; the epoxy resin coating compositions described in U.S. Pat. No. 6,268,055, at col. 2, line 63 to col. 17, line 3; and the alkoxyacrylamide coating compositions descried in U.S. Pat. No. 6,060,001, at col. 2, line 6 to col. 5, line 39. The above-referenced disclosures are hereby specifically incorporated by reference herein.

Further, it will be appreciated by those skilled in the art that the photochromic coating compositions according to various non-limiting embodiments disclosed herein may further comprise other additives that aid in the processing and/or performance of the composition or coating derived therefrom. Non-limiting examples of such additives include photoinitiators, thermal initiators, polymerization inhibitors, solvents, light stabilizers (such as, but not limited to, ultraviolet light absorbers and light stabilizers, such as, hindered amine light stabilizers (HALS)), heat stabilizers, mold release agents, rheology control agents, leveling agents (such as, but not limited to, surfactants), free radical scavengers, adhesion promoters (such as, hexanediol diacrylate and coupling agents), and combinations and mixtures thereof.

According to one non-limiting embodiment, an at least partial coating comprising the photochromic material may be connected to at least a portion of a substrate of a photochromic article, for example, by applying a coating composition comprising the photochromic material to at least a portion of a surface of the substrate and at least partially setting the coating composition. Additionally or alternatively, the at least partial coating comprising the photochromic material may be connected to the substrate, for example, through one or more additional at least partial coatings. For example, while not limiting herein, according to various non-limiting embodiments, an additional coating composition may be applied to a portion of the surface of the substrate, at least partially set, and thereafter a coating composition comprising the photochromic material may be applied over the additional coating and at least partially set. Non-limiting methods of applying coatings compositions to substrates are discussed herein below.

Non-limiting examples of additional coatings and films that may be used in conjunction with the photochromic articles disclosed herein include primer or compatiblizing coatings; protective coatings, including transitional coatings, abrasion-resistant coatings and other coatings that protect against the effects of polymerization reaction chemicals and/or protect against deterioration due to environmental conditions, such as, moisture, heat, ultraviolet light, and/or oxygen (e.g., UV-shielding coatings and oxygen barrier coatings); anti-reflective coatings; conventional photochromic coating; polarizing coatings and polarizing stretched-films; and combinations thereof.

Non-limiting examples of primer or compatiblizing coatings that may be used in conjunction with various non-limiting embodiments disclosed herein include coatings comprising coupling agents, at least partial hydrolysates of coupling agents, and mixtures thereof. As used herein, the term “coupling agent” means a material having a group capable of reacting, binding and/or associating with a group on a surface. Coupling agents according to various non-limiting embodiments disclosed herein may include organometallics, such as, silanes, titanates, zirconates, aluminates, zirconium aluminates, hydrolysates thereof, and mixtures thereof. As used herein, the phrase “at least partial hydrolysates of coupling agents” means that some to all of the hydrolyzable groups on the coupling agent are hydrolyzed. Other non-limiting examples of primer coatings that are suitable for use in conjunction with the various non-limiting embodiments disclosed herein include those primer coatings described U.S. Pat. No. 6,025,026 at col. 3, line 3 to col. 11, line 40 and U.S. Pat. No. 6,150,430 at col. 2, line 39 to col. 7, line 58, which disclosures are hereby specifically incorporated herein by reference.

As used herein, the term “transitional coating” means a coating that aids in creating a gradient in properties between two coatings. For example, although not limiting herein, a transitional coating may aid in creating a gradient in hardness between a relatively hard coating (such as, an abrasion-resistant coating) and a relatively soft coating (such as, a photochromic coating). Non-limiting examples of transitional coatings include radiation-cured, acrylate-based thin films as described in U.S. Patent Application Publication No. 2003/0165686 at paragraphs [0079]-[0173], which disclosure is hereby specifically incorporated by reference herein.

As used herein, the term “abrasion-resistant coating” refers to a protective polymeric material that demonstrates a resistance to abrasion that is greater than a standard reference material, e.g., a polymer made of CR-39® monomer available from PPG Industries, Inc, as tested in a method comparable to ASTM F-735 Standard Test Method for Abrasion Resistance of Transparent Plastics and Coatings Using the Oscillating Sand Method. Non-limiting examples of abrasion-resistant coatings include abrasion-resistant coatings comprising organosilanes, organosiloxanes, abrasion-resistant coatings based on inorganic materials, such as, silica, titania and/or zirconia, and organic abrasion-resistant coatings that are ultraviolet light curable.

Non-limiting examples of antireflective coatings include a monolayer coating or multilayer coatings of metal oxides, metal fluorides, or other such materials, which may be deposited onto the articles disclosed herein (or onto self supporting films that are applied to the articles), for example, through vacuum deposition, sputtering, etc.

Non-limiting examples of polarizing coatings and polarizing stretched-films include, but are not limited to, polarizing coatings (such as those described in U.S. Patent Application Publication No. 2005/0151926, at paragraphs [0029]-[0116], which disclosure is hereby specifically incorporated by reference herein), and polarizing stretched-films comprising dichroic compounds that are known in the art.

As discussed above, according to various non-limiting embodiments an additional at least partial coating or film may be formed on the substrate prior to forming the coating comprising the photochromic material according to various non-limiting embodiments disclosed herein on the substrate. For example, according to certain non-limiting embodiments a primer or compatiblizing coating may be formed on the substrate prior to applying the coating composition comprising the photochromic material. Additionally or alternatively, one or more additional at least partial coating(s) may be formed on the substrate after forming the coating comprising the photochromic material, according to various non-limiting embodiments disclosed herein, on the substrate, for example, as an overcoating on the photochromic coating. For example, according to certain non-limiting embodiments, a transitional coating may be formed over the coating comprising the photochromic material, and an abrasion-resistant coating may then be formed over the transitional coating.

For example, according to certain non-limiting embodiments there is provided a photochromic article comprising a substrate (such as, but not limited to a plano-concave or a plano-convex ophthalmic lens substrate), which comprises an abrasion-resistant coating on at least a portion of a surface thereof; a primer or compatiblizing coating on at least a portion of the abrasion-resistant coating; a photochromic coating comprising a photochromic material, according to various non-limiting embodiments disclosed herein, on at least a portion of the primer or compatiblizing coating; a transitional coating on at least a portion of the photochromic coating; and an abrasion-resistant coating on at least a portion of the transitional coating. Further, according to other non-limiting embodiments, the photochromic article may also comprise, for example, an antireflective coating that is connected to a surface of the substrate and/or a polarizing coating or film that is connected to a surface of the substrate.

One non-limiting embodiment of the present invention provides a method of making a photochromic composition, the method comprising incorporating a photochromic material, according to any of the various non-limiting embodiments of the present invention, into at least a portion of an organic material. Non-limiting methods of incorporating photochromic materials into an organic material include, for example, mixing the photochromic material into a solution or melt of a polymeric or oligomeric material, and subsequently at least partially setting the polymeric or oligomeric material (with or without bonding the photochromic material to the organic material); mixing the photochromic material with a monomeric material and subsequently at least partially polymerizing the monomer (with or without co-polymerizing the photochromic material with the monomer or otherwise bonding the photochromic material to the resultant polymer or intermediate in the polymerization reaction as previously discussed); and imbibing the photochromic material into a polymeric material (with or without bonding the photochromic material to the polymeric material).

Another non-limiting embodiment provides a method of making a photochromic article comprising connecting a photochromic material, according to any of the various non-limiting embodiments discussed above, to at least a portion a substrate. For example, if the substrate is formed from a polymeric material, the photochromic material may be connected to at least a portion of the substrate by the cast-in-place method and/or by imbibition. For example, in the cast-in-place method, the photochromic material may be mixed with a polymeric solution or melt, or other oligomeric and/or monomeric solution or mixture, which may be subsequently cast into a mold having a desired shape and at least partially set to form the substrate. Optionally, according to this non-limiting embodiment, the photochromic material may be bonded to a portion of the polymeric material of the substrate, for example, by co-polymerization with a monomeric precursor thereof or an intermediate in the polymerization reaction. In the imbibition method, the photochromic material may be diffused into the polymeric material of the substrate after it is formed, for example, by immersing a substrate in a solution containing the photochromic material, with or without heating. Thereafter, although not required, the photochromic material may be bonded with the polymeric material.

Other non-limiting embodiments disclosed herein provide methods of making an photochromic article comprising connecting a photochromic material, according to any of the various non-limiting embodiments discussed above, to at least a portion of a substrate by at least one of in-mold casting, coating, and lamination.

For example, according to one non-limiting embodiment wherein the substrate comprises a polymeric material, the photochromic material may be connected to at least a portion of a substrate by in-mold casting. According to this non-limiting embodiment, a coating composition comprising the photochromic material, which may be a liquid coating composition or a powder coating composition, may be applied to the surface of a mold and at least partially set. Thereafter, a polymer solution or melt, or oligomeric or monomeric solution or mixture may be cast over the coating and at least partially set. After setting, the coated substrate may be removed from the mold. Non-limiting examples of powder coatings in which the photochromic materials according to various non-limiting embodiments disclosed herein may be employed are set forth in U.S. Pat. No. 6,068,797 at col. 7, line 50 to col. 19, line 42, which disclosure is hereby specifically incorporated by reference herein.

According to still other non-limiting embodiments, wherein the substrate comprises a polymeric material or an inorganic material, such as, for example, glass, the photochromic material may be connected to at least a portion of a substrate by a coating process. Non-limiting examples of suitable coating processes include spin-coating, spray coating (e.g., using a liquid or a powder coating compositions), curtain coating, roll coating, spin and spray coating, dip coating, over-molding, and combinations thereof. For example, according to one non-limiting embodiment, the photochromic material may be connected to the substrate by over-molding. According to this non-limiting embodiment, a coating composition comprising the photochromic material (examples of which coatings are discussed above) may be applied to a mold and then a substrate may be placed into the mold such that the substrate contacts the coating causing it to spread over at least a portion of the surface of the substrate. Thereafter, the coating composition may be at least partially set and the coated substrate may be removed from the mold. Alternatively, the over-molding process may comprise placing the substrate into a mold such that an open region is defined between the substrate and the mold, and thereafter injecting a coating composition comprising the photochromic material into the open region. Thereafter, the coating composition may be at least partially set and the coated substrate may be removed from the mold. According to another non-limiting embodiment, the photochromic material may be connected to a substrate by spin-coating a coating composition comprising the photochromic material onto the substrate, for example, by rotating the substrate and applying the coating composition to the substrate while it is rotating and/or by applying the coating composition to the substrate and subsequently rotating the substrate.

Additionally or alternatively, a coating composition (with or without a photochromic material) may be applied to a substrate (for example, by any of the foregoing coating processes), the coating composition may be at least partially set, and thereafter, a photochromic material according to any of the various non-limiting embodiments disclosed herein may be imbibed (as previously discussed) into the coating.

As discussed above, according to various non-limiting embodiments disclosed herein, after forming the photochromic coating, at least a portion of the photochromic coating may be at least partially set. For example, according to various non-limiting embodiments disclosed herein, at least partially setting at least a portion of the photochromic coating may comprise exposing the photochromic coating to at least one of electromagnetic radiation and thermal radiation to at least partially dry, polymerize and/or cross-link one or more components of the coating composition.

According to yet another non-limiting embodiment, wherein the substrate comprises a polymeric material or an inorganic material, such as, for example, glass, the photochromic material may be connected to at least a portion of a substrate by lamination. For example, according to this non-limiting embodiment, a self-supporting film or sheet comprising the photochromic material may be adhered or otherwise connected to a portion of the substrate, with or without an adhesive and/or the application of heat and pressure. Optionally, thereafter a protective coating may be applied over the film; or a second substrate may be applied over the first substrate and the two substrates may be laminated together (i.e., by the application of heat and pressure) to form an element wherein the film comprising the photochromic material is interposed between the two substrates. Methods of forming films comprising a photochromic material may include, for example and without limitation, combining a photochromic material with a polymeric or oligomeric solution or mixture, casting or extruding a film therefrom, and, if required, at least partially setting the film. Additionally or alternatively, a film may be formed (with or without a photochromic material) and imbibed with the photochromic material (as discussed above).

Further, according to various non-limiting embodiments, prior to connecting the photochromic material to at least a portion of the substrate by coating or lamination, a primer or compatiblizing coating (such as those discussed above) may be formed on at least a portion of the surface of the substrate to enhance one or more of the wetting, adhesion, and/or chemical compatibility of the photochromic coating with the substrate. Non-limiting examples of suitable primer or compatiblizing coatings and methods of making the same are disclosed above. Still further, as previously discussed according to various non-limiting embodiments disclosed herein, the substrate may comprise an abrasion-resistant coating on at least a portion of its surface.

According to various non-limiting embodiments disclosed herein, prior to applying any coating or film to the substrate, for example, prior to connecting the photochromic material to at least a portion of the surface of the substrate by coating and/or lamination or prior to applying a primer or compatiblizing coating to the substrate, the surface may be cleaned and/or treated to provide a clean surface and/or a surface that may enhance adhesion of the photochromic coating to the substrate. Effective cleaning and treatments may include, but are not limited to, ultrasonic washing with an aqueous soap/detergent solution; cleaning with an aqueous mixture of organic solvent, e.g., a 50:50 mixture of isopropanol:water or ethanol:water; UV treatment; activated gas treatment, e.g., treatment with low temperature plasma or corona discharge; and chemical treatment that results in hydroxylation of the substrate surface, e.g., etching of the surface with an aqueous solution of alkali metal hydroxide, e.g., sodium or potassium hydroxide, which solution can also contain a fluorosurfactant. Generally, the alkali metal hydroxide solution is a dilute aqueous solution, e.g., from 5 to 40 weight percent, more typically from 10 to 15 weight percent, such as, 12 weight percent, alkali metal hydroxide. See, for example, U.S. Pat. No. 3,971,872, column 3, lines 13 to 25; U.S. Pat. No. 4,904,525, column 6, lines 10 to 48; and U.S. Pat. No. 5,104,692, column 13, lines 10 to 59, which describe surface treatments of polymeric organic materials. The foregoing disclosures are specifically incorporated herein by reference.

In one non-limiting embodiment, surface treatment of the substrate may be a low temperature plasma treatment. Although not limiting herein, this method allows treatment of the surface to enhance adhesion of a coating formed thereon, and may be a clean and efficient way to alter the physical surface, e.g., by roughening and/or chemically altering the surface without affecting the rest of the article. Inert gases, such as, argon, and reactive gases, such as, oxygen, may be used as the plasma gas. Inert gases may roughen the surface, while reactive gases, such as, oxygen may both roughen and chemically alter the surface exposed to the plasma, e.g., by producing hydroxyl or carboxyl units on the surface. According to one non-limiting embodiment, oxygen may be used as the plasma gas. Although not limiting herein, it is considered that oxygen may provides a slight, but effective, physical roughening of the surface along with a slight, but effective, chemical modification of the surface. As will be appreciated by those skilled in the art, the extent of the surface roughening and/or chemical modification will be a function of the plasma gas and the operating conditions of the plasma unit (including the length of time of the treatment).

The surface of the substrate subjected to plasma treatment may be at room temperature or may be preheated slightly prior to or during plasma treatment. Although not limiting herein, according to various embodiments, the temperature of the surface to be subjected to a plasma treatment may be maintained at a temperature below a temperature at which the surface may be adversely affected by the plasma (other than the intended increase in surface area by roughening and slight chemical modification). One skilled in the art can readily select the operating conditions of the plasma unit, vis-a-vis, the plastic substrate treated, to achieve an improvement in the adhesion of a superimposed film/coating on the plasma treated surface.

Various non-limiting embodiments disclosed herein further contemplate the use of various combinations of the forgoing methods to form photochromic articles according to various non-limiting embodiments disclosed herein. For example, and without limitation herein, according to one non-limiting embodiment, a photochromic material may be connected to a substrate by incorporation into an organic material from which the substrate is formed (for example, using the cast-in-place method and/or imbibition), and thereafter a photochromic material (which may be the same or different from the aforementioned photochromic material) may be connected to a portion of the substrate using the in-mold casting, coating, and/or lamination methods discussed above.

According to various non-limiting embodiments, the photochromic materials described herein may be used in amounts (or ratios) such that the organic material or substrate into which the photochromic materials are incorporated or otherwise connected exhibits desired optical properties. For example, the amount and types of photochromic materials may be selected such that the organic material or substrate may be substantially clear or colorless when the photochromic material is in the ground-state form and may exhibit a desired resultant color when the photochromic material is in the activated-state form. The precise amount of the photochromic material to be utilized in the various photochromic compositions, photochromic coatings and coating compositions, and photochromic articles described herein is not critical provided that a sufficient amount is used to produce the desired effect. It should be appreciated that the particular amount of the photochromic material used may depend on a variety of factors, such as, but not limited to, the absorption characteristics of the photochromic material, the color and intensity of the color desired upon activation, and the method used to incorporate or connect the photochromic material to the substrate. Although not limiting herein, according to various non-limiting embodiments disclosed herein, the amount of the photochromic material that may be incorporated into an organic material may range from 0.01 to 40 weight percent based on the weight of the organic material.

Various non-limiting embodiments of the present invention will be better understood when read in conjunction with the following non-limiting examples. The procedures set forth in the Examples below are not intended to be limiting herein, as those skilled in the art will appreciate that modifications to the procedures set forth in the Examples, as well as other procedures not described in the Examples, may be useful in preparing photochromic materials according to the present invention.

### EXAMPLES

In part 1 of the Examples, the synthesis procedures used to make photochromic materials according to various non-limiting embodiments disclosed herein are set forth in Examples 1-11 and the procedures used to make comparative photochromic materials are described in Comparative Examples CE1-CE6. In part 2, the preparation of the test chips and test procedures are described. In part 3, the test results are described.

### Part 1: Photochromic Materials—Synthesis

### Example 1

A nitrogen-padded 100 mL 3-necked flask fitted with a reflux condenser, septum and stir bar, was charged with 0.47 gram of 3,3-diphenyl-13-oxo-3H,13H-indeno[2′,3′:3,4]naphtho[1,2-b]pyran and 50 mL of tetrahydrofuran (THF). The solution was stirred and trimethyl(trifluoromethyl)silane (7.5 mL of a 0.5 molar solution in THF) was added drop-wise via syringe. A catalytic amount (10 mg) of tetra-n-butylammonium fluoride was added and the mixture was stirred at room temperature for one day. No reaction was observed, so 8 mL of a fresh solution of trimethyl(trifluoromethyl)silane was added. The mixture was stirred an additional two hours and quenched by pouring the contents of the flask into a beaker containing 50 mL of a 1:1 water:concentrated HCl mixture. After stirring for 12 additional hours, 100 mL of water and 100 mL of THF were added. The organic phase was separated and the aqueous phase was extracted with two additional 50 mL portions of THF. The organic phases were combined, dried, and the solvent removed on a rotary evaporator to give the crude product which was purified by column chromatography on silica gel (hexane:ethyl acetate eluant). The amount of the desired product recovered was 0.37 grams. NMR analysis showed the product to have a structure consistent with 3,3-diphenyl-13-trifluoromethyl-13-hydroxy-3H,13H-indeno[2′,3′:3,4]naphtho[1,2-b]pyran.

### Example 2

The procedure used to prepare Example 1 was followed except that instead of quenching the reaction mixture with a 1:1 mixture of water:concentrated HCl, the crude reaction mixture was stripped of solvent on a rotary evaporator and the residue purified by column chromatography on silica gel (2:1 hexane:ethyl acetate eluant) to recover the desired product. NMR analysis showed the product to have a structure consistent with 3,3-diphenyl-13-trifluoromethyl-13-trimethylsiloxy-3H,13H-indeno[2′,3′:3,4]naphtho[1,2-b]pyran.

### Example 3

PHOTOSOL® 7-114 (2.0 grams; 3,3-di(4-methoxyphenyl)-6,11,13-trimethyl-13-hydroxy-3H,13H-indeno[2′,3′:3,4]naphtho[1,2-b]pyran; commercially available from PPG Industries, Inc., Pittsburgh, Pa.) was added to a reaction flask containing 15 mL of 2,2,2-trifluoroethanol and 40 mL of acetonitrile. The resulting mixture was stirred under a nitrogen atmosphere and heated to 80° C. Subsequently, 0.1 grams of p-toluenesulfonic acid was added to the reaction mixture. After 5 minutes at 80° C., the reaction was quenched into 600 mL of water with vigorous stirring upon which a greenish-yellow solid precipitated out. The solid was filtered, washed with water, and dried open to air. This solid was purified by column chromatography. Subsequent crystallization from a diethyl ether and hexane mixture yielded 80 mg of a white solid. NMR analysis showed the product to have a structure consistent with 3,3-di(4-methoxyphenyl)-6,11,13-trimethyl-13-(2,2,2-trifluoroethoxy)-3H,13H-indeno┌2′,3′:3,4┐naphtho┌1,2-b┐pyran.

### Example 4

3,3,-Di(4-methoxyphenyl)-6-methoxy-7-morpholino-13-ethyl-13-hydroxy-3H,13H-indeno[2′,3′:3,4]naphtho[1,2-b]pyran (1.85 grams), 2,2,2-trifluoroethanol (20 mL), toluene (30 mL), and methanesulfonic acid (15 drops) were combined in a reaction flask and heated to 75° C. for 7 hours. The reaction mixture was cooled to room temperature, diluted with 100 mL of toluene, and then washed with a 1:1 saturated sodium bicarbonate:water mixture (2×200 mL). The organic layer was removed and concentrated by rotary evaporation to give a dark colored solid. This solid was purified by column chromatography. NMR analysis showed the product to have a structure consistent with 3,3-di(4-methoxyphenyl)-6-methoxy-7-morpholino-13-ethyl-13-(2,2,2-trifluoroethoxy)-3H,13H-indeno[2′,3′:3,4]naphtho[1,2-b]pyran.

### Example 5

Step 1

Anisole (27.5 grams), 4-fluorobenzoyl chloride (35 grams) and dichloromethane (250 mL) were combined in a reaction flask. Aluminum chloride (30.8 grams) was added to the reaction mixture slowly over 20 minutes. The reaction mixture was stirred at room temperature for two hours and then poured into a mixture of 70 mL concentrated HCl and 500 mL of water. The layers were separated and the aqueous layer was extracted with two portions of dichloromethane (300 mL each). The organic phases were combined and washed with saturated aqueous sodium bicarbonate (400 mL). The organic layer was dried over anhydrous sodium sulfate, filtered, and the filtrate was evaporated to yield 48.0 grams of 4-fluoro-4′-methoxybenzophenone as a white solid. This material was not purified further but was used directly in the next step.

Step 2

4-Fluoro-4′-methoxybenzophenone from Step 1 (126.7 grams) and acetylene saturated N,N-dimethylformamide (380 mL) were combined in a reaction flask. Sodium acetylide solution (9% by weight in toluene, 343 grams) was added to the reaction mixture dropwise over 45 minutes. The reaction mixture was stirred at room temperature for 1 hour and then poured into ice water (600 mL). The layers were separated and the aqueous layer was extracted with three portions of diethyl ether (200 mL). The organic layers were combined and washed with saturated aqueous ammonium chloride (200 mL), saturated aqueous sodium chloride (200 mL), and saturated aqueous sodium bicarbonate (200 mL). The organic layer was dried over anhydrous sodium sulfate, filtered, and the filtrate was evaporated to an amber colored oil yielding 136.6 grams of 1-(4-fluorophenyl)-1-(4-methoxyphenyl)-2-propyn-1-ol. This material was not purified further but was used directly in the next step.

Step 3

2,3-Dimethoxy-5-acetoxy-7H-benzo[C]fluoren-7-one (50.0 grams) was stirred in anhydrous THF (1000 mL) in an oven-dried reaction flask under a nitrogen atmosphere. The reaction flask was placed in a dry ice/acetone bath and a 2.5 M solution of n-butyllithium in hexanes (215 mL) was added dropwise to the reaction over 30 minutes. The reaction mixture was slowly warmed to room temperature and subsequently poured into a saturated aqueous ammonium chloride and ice mixture (1 L). The layers were separated and the aqueous layer was extracted with two 600 mL portions of ethyl acetate. The organic portions were combined and washed with saturated aqueous sodium bicarbonate (1 L). The organic layer was dried over magnesium sulfate, filtered, and the filtrate was concentrated by rotary evaporation. The resulting brown solid was slurried in hot t-butyl methyl ether, cooled to room temperature, collected by vacuum filtration, and washed with cold t-butyl methyl ether to yield 34.9 grams of 2,3-dimethoxy-5,7-dihydroxy-7-butyl-7II-benzo[C]fluorene. The product was used without further purification in the next step.

Step 4

2,3-Dimethoxy-5,7-dihydroxy-7-butyl-7H-benzo┌C┐fluorene (30.0 grams) from Step 3, morpholine (43 mL), and anhydrous THF (900 mL) were combined and stirred in a reaction flask fitted with a reflux condenser under a nitrogen atmosphere. The reaction flask was placed in an ice bath and a solution of n-butyllithium (2.5 M in hexanes, 165 mL) was added dropwise to the reaction over 30 minutes. The reaction mixture was heated to reflux for 3 hours and subsequently cooled to room temperature and poured into a saturated aqueous ammonium chloride and ice mixture (1 L). The layers were separated and the aqueous layer was extracted with two 600 mL portions of ethyl acetate. The combined organic portions were washed with saturated aqueous sodium bicarbonate (700 mL), dried over magnesium sulfate, filtered, and the filtrate was concentrated by rotary evaporation. The resulting orange solid was slurried in hot 15% hexanes/85% t-butyl methyl ether, cooled to room temperature, collected by vacuum filtration, and washed with cold 15% hexanes/85% t-butyl methyl ether to yield 23.9 grams of 2-morpholino-3-methoxy-5,7-dihydroxy-7-butyl-7H-benzo[C]fluorene. The product was used without further purification in the next step.

Step 5

1-(4-Fluorophenyl)-1-(4-methoxyphenyl)-2-propyn-1-ol from Step 2 (7.9 grams), 2-morpholino-3-methoxy-5,7-dihydroxy-7-butyl-7H-benzo[C]fluorene from Step 4 (10.0 grams), and chloroform (200 mL, preserved with pentene) were combined and stirred in a reaction flask. Trifluoroacetic acid (680 mg) was added to the reaction followed by dodecylbenzenesulfonic acid (1.95 grams). After 24 hours, an additional 1-(4-fluorophenyl)-1-(4-methoxyphenyl)-2-propyn-1-ol (1.0 gram) was charged to the reaction mixture. The reaction mixture was stirred for an additional 24 hours and then washed with saturated aqueous sodium bicarbonate (150 mL), filtered through celite, and the layers separated. The organic layer was dried over magnesium sulfate, filtered, and the filtrate was concentrated by rotary evaporation. The resulting dark colored oil was purified by column chromatography on silica gel (250 grams) eluting with 30% ethyl acetate/70% hexanes. The fractions containing pure product were combined and evaporated to afford an orange solid. The solid was slurried in methanol and then filtered yielding 7.5 grams of 3-(4-fluorophenyl)-3-(4-methoxyphenyl)-6-methoxy-7-morphlino-13-butyl-13-hydroxy-3H,13H-indeno[2′,3′:3,4]naphtho[1,2-b]pyran.

Step 6

3-(4-Fluorophenyl)-3-(4-methoxyphenyl)-6-methoxy-7-morphlino-13-butyl-13-hydroxy-3H,13H-indeno└2′,3′:3,4┘naphtho└1,2-b┘pyran (1.5 grams) from Step 5, 1H,1H,2H,2H-perfluorododecan-1-ol (6 grams), p-toluenesulfonic acid monohydrate (110 mg), and toluene (9 mL) were combined in a reaction flask under a nitrogen atmosphere and heated to 85° C. for 5 hours. The reaction mixture was cooled to room temperature and filtered. The filtrate was washed with saturated aqueous sodium bicarbonate (15 mL). The layers were separated and the organic layer was dried over magnesium sulfate, filtered, and the filtrate was concentrated by rotary evaporation. The resulting oil was purified by column chromatography on silica gel (200 grams) eluting with 15% ethyl acetate/85% hexanes. The fractions containing product of good purity were combined and concentrated by rotary evaporation resulting in a white solid. The solid was slurried in hot methanol and then filtered off yielding 1.9 grams of a white solid. NMR analysis showed the product structure consistent with 3-(4-fluorophenyl)-3-(4-methoxyphenyl)-6-methoxy-7-morpholino-13-butyl-13-(1H,1H,2H,2H-perfluorododecanoxy)-3H,13H-indeno┌2′,3′:3,4┐naphtho┌1,2-b┐pyran.

### Example 6

3-(4-Fluorophenyl)-3-(4-methoxyphenyl)-6-methoxy-7-morphlino-13-butyl-13-hydroxy-3H,13H-indeno[2′,3′:3,4]naphtho[1,2-b]pyran (2.9 grams) from Step 5 of Example 5, 3-(perfluorobutyl)-1-propanol (5 grams), p-toluenesulfonic acid monohydrate (210 mg), and toluene (30 mL) were combined in a reaction flask and heated to 75° C. for 4 hours. The reaction mixture was cooled to room temperature and washed with saturated aqueous sodium bicarbonate (20 mL). The layers were separated and the organic layer was dried over magnesium sulfate, filtered, and the filtrate was concentrated by rotary evaporation. The resulting oil was purified by column chromatography on silica gel (280 grams) eluting with 20% ethyl acetate/80% hexanes. The fractions containing product of good purity were combined and concentrated by rotary evaporation. The resulting green oil was placed under hi-vacuum and subsequently foamed up to yield 3.2 grams of a green foam. NMR analysis showed the product to have a structure consistent with 3-(4-fluorophenyl)-3-(4-methoxyphenyl)-6-methoxy-7-morpholino-13-butyl-13-(3-perfluorobutylpropoxy)-3H,13H-indeno[2′,3′:3,4]naphtho[1,2-b]pyran.

### Example 7

A nitrogen-padded 100 mL 3-necked flask fitted with a reflux condenser, septum and stir bar, was charged with 1.05 grams of 3,3-di(4-methoxyphenyl)-6,11-dimethyl-13-oxo-3H,13H-indeno[2′,3′:3,4]naphtho[1,2-b]pyran and 50 mL of THF. The solution was stirred and trimethyl(trifluoromethyl)silane (8 mL of a 0.5 molar solution in THF) was added dropwise via syringe. A catalytic amount (10 mg) of tetra-n-butylammonium fluoride was added and the mixture was stirred at room temperature for two days. During this period, the solution went from a red to a light blue coloration. The reaction was quenched by pouring the contents of the flask into a beaker containing 50 mL of a 1:1 water:concentrated HCl mixture. After stirring for 12 additional hours, 100 mL of water and 100 mL of THF were added. The organic phase was separated and the aqueous phase was extracted with two additional 50 mL portions of THF. The organic phases were combined, dried, and the solvent was removed on a rotary evaporator to give the crude product which was purified by column chromatography on silica gel (hexane:ethyl acetate eluant). Collection of the photochromic fractions yielded 260 mg of the desired product. NMR analysis showed the product to have a structure consistent with 3,3-di(4-methoxyphenyl)-6,11-dimethyl-13-trifluoromethyl-13-hydroxy-3H,13H-indeno[2′,3′:3,4]naphtho[1,2-b]pyran.

### Example 8

3,3,-Di(4-methoxyphenyl)-6-methoxy-7-morpholino-13-ethyl-13-hydroxy-3H, 13H-indeno[2,1-f]naphtho[1,2-b]pyran (3.25 grams), 2,2,3,3,3-pentafluoropropanol (25 mL), toluene (50 mL), and methane sulfonic acid (14 drops) were combined in a reaction flask and heated to 75° C. for 5 hours. The reaction mixture was cooled to room temperature, diluted with toluene (100 mL), and washed with 150 mL of a 1:1 saturated sodium bicarbonate/water mixture. The solvent was removed by rotary evaporation and give a dark colored solid. This solid was purified by column chromatography to give 2.8 grams of pure product. NMR analysis showed the product to have a structure consistent with 3,3-di(4-methoxyphenyl)-6-methoxy-7-morpholino-13-ethyl-13-(2,2,3,3,3-pentafluoropropoxy)-3H,13H-indeno[2′,3′:3,4]naphtho[1,2-b]pyran.

### Example 9

3,3,-Di(4-methoxyphenyl)-6-methoxy-7-morpholino-13-ethyl-13-hydroxy-3H, 13H-indeno[2′,3′:3,4]naphtho[1,2-b]pyran (3.15 grams), 2,2,3,3,4,4,4,-heptafluorobutanol (30 mL), toluene (50 mL), and methane sulfonic acid (12 drops) were combined in a reaction flask and heated to 75° C. for 3 hours. The reaction mixture was cooled to room temperature, diluted with toluene (100 mL), and washed with 150 mL of a 1:1 saturated sodium bicarbonate/water mixture. The solvent was removed by rotary evaporation and give a dark colored solid. This solid was purified by column chromatography to 3.6 grams of pure product. NMR analysis showed the product to have a structure consistent with 3,3-di(4-methoxyphenyl)-6-methoxy-7-morpholino-13-ethyl-13-(2,2,3,3,4,4,4,-heptafluorobutoxy)-3H,13H-indeno[2′,3′:3,4]naphtho[1,2-b]pyran.

### Comparative Example CE1

PHOTOSOL®7-114 (2.0 grams), anhydrous methanol (20 mL), toluene (20 mL), and p-toluenesulfonic acid monohydrate (0.2 grams) were combined in a reaction flask and heated to reflux. The reaction mixture was refluxed overnight, cooled to room temperature and diluted with toluene (100 mL). Reaction mixture was washed with 50% saturated aqueous sodium bicarbonate (200 mL). The organic phase was separated, dried over anhydrous sodium sulfate, filtered, and the filtrate was concentrated by rotary evaporation. The resulting residue was purified by column chromatography on silica gel. The photochromic fractions were combined and concentrated by rotary evaporation to obtain 1.8 grams of a tan solid. Mass spectrometry and NMR analysis showed the product to have a structure consistent with 3,3-di(4-methoxyphenyl)-6,11,13-trimethyl-13-methoxy-3H,13H-indeno[2′,3′:3,4]naphtho[1,2-b]pyran.

### Comparative Example CE2

Step 1

2,3-Dimethoxy-5-acetoxy-7H-benzo[C]fluoren-7-one (13.5 grams) was stirred in anhydrous THF (185 mL) in an oven-dried reaction flask under a nitrogen atmosphere. The reaction flask was placed in an ice bath and a 3.0 M solution of ethylmagnesium bromide in diethyl ether (215 mL) was added dropwise over 30 minutes. The reaction mixture was slowly warmed to room temperature and then poured into a saturated aqueous ammonium chloride and ice mixture (300 mL). The layers were separated and the aqueous layer was extracted with three 125 mL portions of ethyl acetate. The organic portions were combined and washed with saturated aqueous sodium bicarbonate (500 mL). The organic layer was dried over magnesium sulfate, filtered, and the filtrate was concentrated by rotary evaporation. The resulting brown solid was slurried in acetonitrile and collected by vacuum filtration, yielding 7.3 grams of 2,3-dimethoxy-5,7-dihydroxy-7-ethyl-7H-benzo[C]fluorene. The product was used without further purification in the subsequent reaction.

Step 2

2,3-Dimethoxy-5,7-dihydroxy-7-ethyl-7H-benzo└C┘fluorene (20.0 grams) from Step 1, morpholine (31 mL), and anhydrous THF (600 mL) were combined and stirred in a reaction flask under a nitrogen atmosphere. The reaction flask was placed in an ice bath and a 2.5 M solution of n-butyllithium in hexanes (120 mL) was added dropwise over 30 minutes. The reaction mixture was heated to reflux for 2.5 hours, cooled to room temperature and poured into a saturated aqueous ammonium chloride and ice mixture (600 mL). The layers were separated and the aqueous layer was extracted with two 400 mL portions of ethyl acetate. The organic portions were combined and washed with saturated aqueous sodium bicarbonate (500 mL). The layers were separated and the organic layer was dried over magnesium sulfate, filtered, and the filtrate was concentrated by rotary evaporation. The resulting orange solid was slurried in hot 50% hexanes/50% t-butyl methyl ether, cooled to room temperature, collected by vacuum filtration, and washed with cold 50% hexanes/50% t-butyl methyl ether to yield 18.4 grams of 2-morpholino-3-methoxy-5,7-dihydroxy-7-ethyl-7H-benzo[C]fluorene. The product was used without further purification in the subsequent reaction.

Step 3

1-(4-fluorophenyl)-1-(4-methoxyphenyl)-2-propyn-1-ol from Step 2 of Example 5 (8.5 grams), 2-morpholino-3-methoxy-5,7-dihydroxy-7-ethyl-7H-benzo[C]fluorene from Step 4 (10.0 grams), and pentene preserved chloroform (200 mL) were combined and stirred in a reaction flask. Trifluoroacetic acid (730 mg) was added to the reaction mixture followed by dodecylbenzenesulfonic acid (2.1 grams). The reaction mixture was heated to 40° C. for 7 hours and then cooled to room temperature. The reaction mixture was washed with saturated aqueous sodium bicarbonate (250 mL), filtered through celite, and the layers were separated. The organic layer was dried over magnesium sulfate, filtered, and filtrate was concentrated by rotary evaporation. The resulting dark colored oil was recrystallized in 50% toluene/50% hexanes and collected by vacuum filtration yielding 11.2 grams of a red crystalline solid. Mass Spectrometry and NMR analysis show the product to have a structure consistent with 3-(4-fluorophenyl)-3-(4-methoxyphenyl)-6-methoxy-7-morpholino-13-ethyl-13-hydroxy-3H, 13H-indeno┌2′,3′:3,4┐naphtho┌1,2-b┐pyran.

### Comparative Example CE3

3,3-di(4-methoxyphenyl)-6,11,13-trimethyl-13-hydroxy-3H,13H-indeno[2′,3′:3,4]naphtho[1,2-b]pyran is a white powder that is commercially available as PHOTOSOL® 7-114 from PPG Industries, Inc., Pittsburgh, Pa.

### Comparative Example CE4

3,3-Di(4-methoxyphenyl)-6-methoxy-7-morpholino-13-ethyl-13-hydroxy-3H,13H-indeno[2′,3′:3,4]naphtho[1,2-b]pyran (68.7 grams), anhydrous methanol (685 mL), toluene (685 mL), and p-toluenesulfonic acid monohydrate (5.1 grams) were combined in a reaction flask and heated to reflux. Additional p-toluenesulfonic acid monohydrate was charged in two 0.5 gram portions; after refluxing for four hours, and then again after eight hours. The reaction mixture was then refluxed overnight, cooled to room temperature, diluted with toluene (400 mL), and washed with 50% saturated aqueous sodium bicarbonate (800 mL). The organic layer was separated, dried over anhydrous sodium sulfate, filtered, and the filtrate was concentrated by rotary evaporation. The resulting residue was purified by column chromatography on silica gel (1,300 grams) eluting with 25% ethyl acetate in hexanes. The photochromic fractions were combined and concentrated by rotary evaporation. The resulting residue was recrystallized in 20% hexanes in t-butyl methyl ether yielding 62.6 grams of a tan solid. Mass Spectrometry and NMR analysis showed the product to have a structure consistent with 3,3-di(4-methoxyphenyl)-6-methoxy-7-morpholino-13-ethyl-13-methoxy-3H,13H-indeno[2′,3′:3,4]naphtho[1,2-b]pyran.

### Comparative Example CE5

3-(4-Fluorophenyl)-3-(4-methoxyphenyl)-6-methoxy-7-morpholino-13-ethyl-13-hydroxy-3H,13H-indeno[2′,3′:3,4]naphtho[1,2-b]pyran from Step 3 of Comparative Example CE2 (0.76 grams), p-toluenesulfonic acid monohydrate (137 mg), anhydrous methanol (10 mL), and toluene (10 mL) were combined in a reaction flask under a nitrogen atmosphere and heated to reflux for 13 hours. The reaction mixture was cooled to room temperature and washed with two 10 mL portions of saturated aqueous sodium bicarbonate. The organic layer was dried over magnesium sulfate, filtered, and the filtrate was concentrated by rotary evaporation. The resulting oil was recrystallized in methanol and collected by vacuum filtration yielding 0.65 grams of a yellow solid. Mass Spectrometry and NMR analysis showed the product to have a structure consistent with 3-(4-fluorophenyl)-3-(4-methoxyphenyl)-6-methoxy-7-morpholino-13-ethyl-13-methoxy-3H,13H-indeno[2′,3′:3,4]naphtho[1,2-b]pyran.

### Comparative Example CE6

3,3-Diphenyl-13-butyl-13-hydroxy-3H,13H-indeno[2′,3′:3,4]naphtho[1,2-b]pyran was prepared following the procedure of Examples 1 and 2 of U.S. Pat. No. 5,645,767 except that in Step 6 of Example 1, 1,1-diphenyl-2-propyn-1-ol was used in place of 1,1-di(4-methoxyphenyl)-2-propyn-1-ol, and in Example 2, n-butyl Grignard instead of methyl Grignard was used.

### Part 2: Photochromic Coating/Chip Preparation

The photochromic performance of the photochromic materials of Examples 1-11 and Comparative Examples 1-6 were tested as follows. A quantity of the photochromic material to be tested, calculated to yield a 1.5×10⁻³ M solution, was added to a flask containing 50 grams of a monomer blend of 4 parts ethoxylated bisphenol A dimethacrylate (BPA 2EO DMA), 1 part poly(ethylene glycol) 600 dimethacrylate, and 0.033 weight percent 2,2′-azobis(2-methyl propionitrile) (AIBN). The photochromic material was dissolved into the monomer blend by stirring and gentle heating. After a clear solution was obtained, it was vacuum degassed before being poured into a flat sheet mold having the interior dimensions of 2.2 mm×6 inches (15.24 cm)×6 inches (15.24 cm). The mold was sealed and placed in a horizontal air flow, programmable oven programmed to increase the temperature from 40° C. to 95° C. over a 5 hour interval, hold the temperature at 95° C. for 3 hours and then lower it to 60° C. for at least 2 hours. After the mold was opened, the polymer sheet was cut using a diamond blade saw into 2 inch (5.1 cm) test squares.

The photochromic test squares prepared as described above were tested for photochromic response on an optical bench. Prior to testing on the optical bench, the photochromic test squares were exposed to 365 nm ultraviolet light for about 15 minutes to cause the photochromic material to transform from the ground state-form to an activated-state form, and then placed in a 75° C. oven for about 15 minutes to allow the photochromic material to revert back to the ground state-form. The test squares were then cooled to room temperature, exposed to fluorescent room lighting for at least 2 hours, and then kept covered (that is, in a dark environment) for at least 2 hours prior to testing on an optical bench maintained at 73° F. (23° C.). The bench was fitted with a 300-watt xenon arc lamp, a remote controlled shutter, a Melles Griot KG2 filter that modifies the UV and IR wavelengths and acts as a heat-sink, neutral density filter(s) and a sample holder, situated within a water bath, in which the square to be tested was inserted. A collimated beam of light from a tungsten lamp was passed through the square at a small angle (approximately 30°) normal to the square. After passing through the square, the light from the tungsten lamp was directed to a collection sphere, where the light was blended, and on to an Ocean Optics S2000 spectrometer where the spectrum of the measuring beam was collected and analyzed. The λₘₐₓ₋ᵥᵢₛ the wavelength in the visible spectrum at which the maximum absorption of the activated-state form of the photochromic compound in a test square occurs. The λₘₐₓ₋ᵥᵢₛ wavelength was determined by testing the photochromic test squares in a Varian Cary 300 UV-Visible spectrophotometer; it may also be calculated from the spectrum obtained by the S2000 spectrometer on the optical bench.

The saturated optical density (“Sat'd OD”) for each test square was determined by opening the shutter from the xenon lamp and measuring the transmittance after exposing the test chip to UV radiation for 30 minutes. The λₘₐₓ₋ᵥᵢₛ at the Sat'd OD was calculated from the activated data measured by the S2000 spectrometer on the optical bench. The First Fade Half Life (“T_{1/2}”) is the time interval in seconds for the absorbance of the activated-state form of the photochromic material in the test squares to reach one half the Sat'd OD absorbance value at room temperature (73° F.), after removal of the source of activating light.

### Part 3: Testing and Results

Results for the photochromic materials tested are listed below in Table 1.

TABLE 1
Photochromic Test Data

| | | λₘₐₓ₋ᵥᵢₛ | | T_{1/2} |
|---|---|---|---|---|
| No. | Example | (nm) | Sat'd OD | (sec) |
| 1 | 3,3-diphenyl-13-hydroxy-13-trifluoromethyl- | 529 | 0.45 | 63 |
| | 3H,13H-indenoL[2′,3′:3,4]naphtho[1,2-b]pyran | | | |
| 2 | 3,3-diphenyl-13-trimethylsilyloxy-13- | 525 | 0.77 | 112 |
| | trifluoromethyl-3H,13H-indeno[2′,3′:3,4]- | | | |
| | naphtha[1,2-b]pyran | | | |
| 3 | 3,3-di(4-methoxyphenyl)-6,11,13-trimethyl-13- | 570 | 0.54 | 88 |
| | (2,2,2-trifluoroethoxy)-3H,13H-indeno- | | | |
| | [2′,3′:3,4]naphtho[1,2-b]pyran | | | |
| 4 | 3,3-di(4-methoxyphenyl)-6-methoxy-7- | 479 | 0.63 | 80 |
| | morpholino-13-ethyl-13-(2,2,2-trifluoroethoxy)- | | | |
| | 3H,13H-indeno[2′,3′:3,4]naphtho[1,2-b]pyran | | | |
| 5 | 3-(4-fluorophenyl)-3-(4-methoxyphenyl)-6- | 472 | 1.56 | 182 |
| | methoxy-7-morpholino-13-butyl-13- | | | |
| | (1H,1H,2H,2H-perfluorododecanoxy)-3H,13H- | | | |
| | indeno [2′,3′:3,4]naphtho[1,2-b]pyran | | | |
| 6 | 3-(4-fluorophenyl)-3-(4-methoxyphenyl)-6- | 465 | 1.84 | 173 |
| | methoxy-7-morpholino-13-butyl-13-(3- | | | |
| | perfluorobutylpropoxy)-3H,13H-indeno- | | | |
| | [2′,3′:3,4]naphtho[1,2-b]pyran | | | |
| 7 | 3,3-di(4-methoxyphenyl)-6,11-dimethyl-13- | 563 | 0.14 | 35 |
| | trimethylsilyloxy-13-trifluoromethyl-3H,13H- | | | |
| | indeno[2′,3′:3,4]naphtho[1,2-b]pyran | | | |
| 8 | 3,3-di(4-methoxyphenyl)-6-methoxy-7- | 481 | 0.69 | 97 |
| | morpholino-13-ethyl-13-(2,2,3,3,3-pentafluoropropoxy)- | | | |
| | 3H,13H-indeno[2′,3′:3,4]naphtho- | | | |
| | [1,2-b]pyran | | | |
| 9 | 3,3-di(4-methoxyphenyl)-6-methoxy-7- | 481 | 0.75 | 101 |
| | morpholino-13-ethyl-13-(2,2,3,3,4,4,4-heptafluorobutoxy)- | | | |
| | 3H,13H-indeno[2′,3′:3,4] | | | |
| | naphtha[1,2-b]pyran | | | |
| 10 | 3-(4-fluorophenyl)-3-(4-methoxyphenyl)-6- | 475 | 1.03 | 128 |
| | methoxy-7-morpholino-13-butyl-13-(2,2,2- | | | |
| | trifluoroethoxy)-3H,13H-indeno[2′,3′:3,4]- | | | |
| | naphtha[1,2-b]pyran | | | |
| 11 | 3-(4-fluorophenyl)-3-(4-methoxyphenyl)-6- | 474 | 1.00 | 128 |
| | methoxy-7-morpholino-13-ethyl-13-(2,2,2- | | | |
| | trifluoroethoxy)-3H,13H-indeno[2′,3′:3,4]- | | | |
| | naphtha[1,2-b]pyran | | | |
| CE1 | 3,3-di(4-methoxyphenyl)-6,11,13-trimethyl-13- | 570 | 0.72 | 144 |
| | methoxy-3H,13H-indeno[2′,3′:3,4]- | | | |
| | naphtha[1,2-b]pyran | | | |
| CE2 | 3-(4-fluorophenyl)-3-(4-methoxyphenyl)-6- | 468 | 1.09 | 172 |
| | methoxy-7-morpholino-13-ethyl-13-hydroxy- | | | |
| | 3H,13H-indeno[2′,3′:3,4]naphtho[1,2-b]pyran | | | |
| CE3 | 3,3-di(4-methoxyphenyl)-13-hydroxy-6,11,13- | 575 | 0.50 | 119 |
| | trimethyl-3H,13H-indeno[2′,3′:3,4]- | | | |
| | naphtha[1,2-b]pyran | | | |
| CE4 | 3,3-di(4-methoxyphenyl)-6-methoxy-7- | 474 | 0.82 | 135 |
| | morpholino-13-ethyl-13-methoxy-3H,13H- | | | |
| | indeno[2′,3′:3,4]naphtho[1,2-b]pyran- | | | |
| CE5 | 3-(4-fluorophenyl)-3-(4-methoxyphenyl)-6- | 470 | 1.41 | 160 |
| | methoxy-7-morpholino-13-ethyl-13-methoxy- | | | |
| | 3H,13H-indeno[2′,3′:3,4]naphtho[1,2-b]pyran | | | |
| CE6 | 3,3-diphenyl-13-butyl-13-hydroxy-3H,13H- | 541 | 0.67 | 174 |
| | indeno[2′,3′:3,4]naphtho[1,2-b]pyran | | | |

The photochromic performance results in Table 1 for photochromic materials according to various non-limiting embodiments of the present disclosure may be compared with the performance results of related compounds in the Comparative Examples which lack the claimed features of the photochromic materials of the present disclosure. For example, comparison of the fade rates for Example 1 (i.e., 3,3-diphenyl-13-hydroxy-13-trifluoromethyl-3H,13H-indeno[2′,3′:3,4]naphtho[1,2-b]pyran) and Example 2 (i.e., 3,3-diphenyl-13-trimethylsilyloxy-13-trifluoromethyl-3H,13H-indeno┌2′,3′:3,4┐naphtho┌1,2-b]pyran) to Comparative Example CE6 (i.e., 3,3-diphenyl-13-butyl-13-hydroxy-3H,13H-indeno[2′,3′:3,4]naphtho[1,2-b]pyran), it can be seen that each of the photochromic materials of Examples 1 and 2 (T_{1/2} value of 63 seconds and 112 seconds, respectively) have faster fade rates (i.e., smaller T_{1/2} values) than Comparative Example CE6 (T_{1/2} value of 174 seconds). Comparison of the fade rates of Example 3 (i.e., 3,3-di(4-methoxyphenyl)-6,11,13-trimethyl-13-(2,2,2-trifluoroethoxy)-3II,13II-indeno[2′,3′:3,4]naphtho[1,2-b]pyran) and Example 7 (i.e., 3,3-di(4-methoxyphenyl)-6,11-dimethyl-13-trimethylsilyloxy-13-trifluoromethyl-3H,13H-indeno┌2′,3′:3,4┐naphtho┌1,2-b┐pyran) to Comparative Example CE1 (i.e., 3,3-di(4-methoxyphenyl)-6,11,13-trimethyl-13-methoxy-3H,13H-indeno[2′,3′:3,4]naphtho[1,2-b]pyran) and Comparative Example CE3 (i.e., 3,3-di(4-methoxyphenyl)-13-hydroxy-6,11,13-trimethyl-3H,13H-indeno[2′,3′:3,4]naphtho[1,2-b]pyran) show that each of the photochromic materials of Examples 3 and 7 (T_{1/2} values of 88 seconds and 35 seconds, respectively) has a faster fade rate than either of Comparative Examples CE1 and CE3 (T_{1/2} values of 144 seconds and 119 seconds, respectively). Comparison of the fade rates of Example 4 (i.e., 3,3-di(4-methoxyphenyl)-6-methoxy-7-morpholino-13-ethyl-13-(2,2,2-trifluoroethoxy)-3H,13H-indeno[2′,3′:3,4]naphtho[1,2-b]pyran), Example 8 (i.e., 3,3-di(4-methoxyphenyl)-6-methoxy-7-morpholino-13-ethyl-13-(2,2,3,3,3-pentafluoropropoxy)-3H,13H-indeno[2′,3′:3,4]naphtho[1,2-b]pyran) and Example 9 (i.e., 3,3-di(4-methoxyphenyl)-6-methoxy-7-morpholino-13-ethyl-13-(2,2,3,3,4,4,4-heptafluorobutoxy)-3H,13H-indeno[2′,3′:3,4]naphtho[1,2-b]pyran) to Comparative Example CE4 (i.e., 3,3-di(4-methoxyphenyl)-6-methoxy-7-morpholino-13-ethyl-13-methoxy-3H,13H-indeno[2′,3′:3,4]naphtho[1,2-b]pyran) shows that each of the photochromic materials of Examples 4, 8, and 9 (T_{1/2} values of 80 seconds, 97 seconds, and 101 seconds, respectively) has a faster fade rate than Comparative Example CE4 (T_{1/2} value of 135 seconds). In addition, comparison of the fade rates of Example 11 (i.e., 3-(4-fluorophenyl)-3-(4-methoxyphenyl)-6-methoxy-7-morpholino-13-ethyl-13-(2,2,2-trifluoroethoxy)-3H,13H-indeno[2′,3′:3,4]naphtho[1,2-b]pyran) to Comparative Example CE2 (i.e., 3-(4-fluorophenyl)-3-(4-methoxyphenyl)-6-methoxy-7-morpholino-13-ethyl-13-hydroxy-3H,13H-indeno┌2′,3′:3,4┐naphtho┌1,2-b┐pyran) and Comparative Example CE5 (i.e., 3-(4-fluorophenyl)-3-(4-methoxyphenyl)-6-methoxy-7-morpholino-13-ethyl-13-methoxy-3H,13H-indeno[2′,3′:3,4]naphtho[1,2-b]pyran) show that the photochromic material of Example 11 (T_{1/2} value of 128 seconds) has a faster fade rate than either of Comparative Examples CE2 and CE5 (T_{1/2} values of 172 seconds and 160 seconds, respectively).

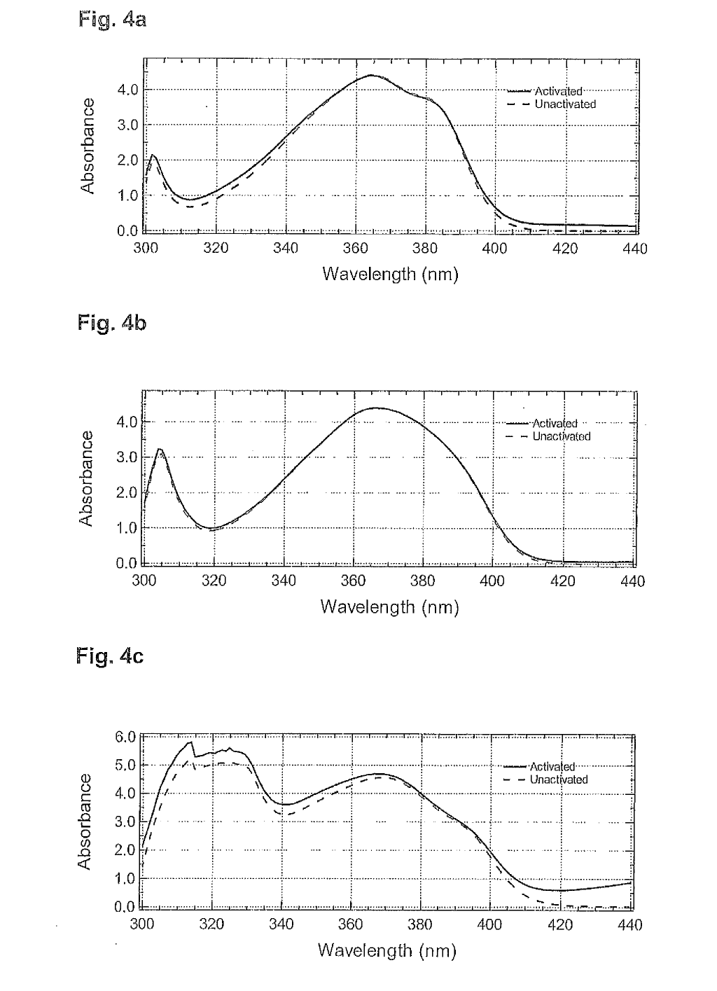

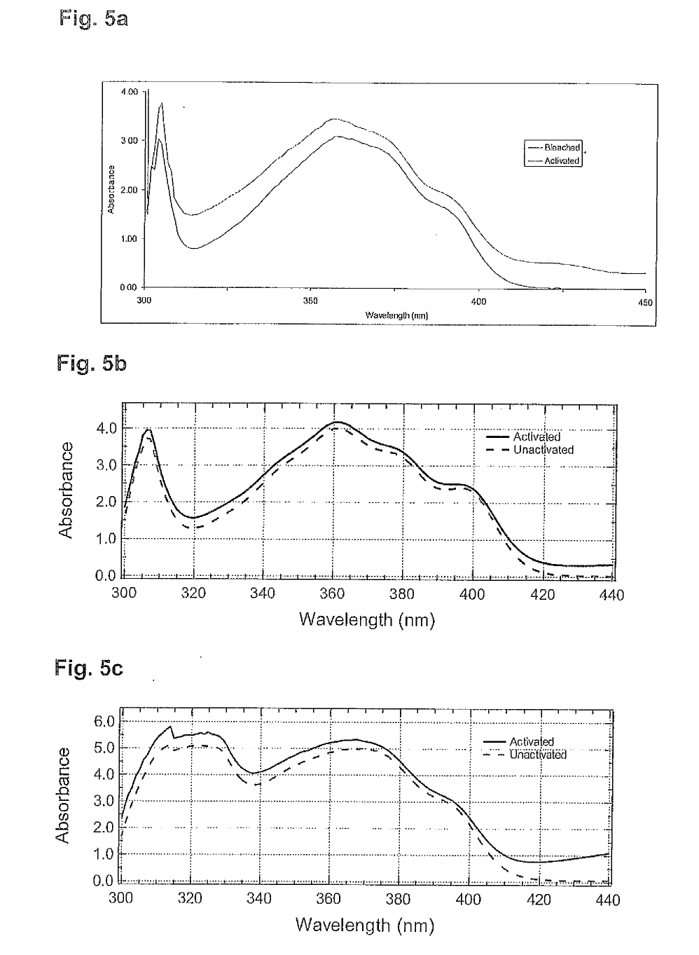

Referring now to FIGS. 4 and 5, the hypsochromic shift of the closed-form (unactivated or bleached form) absorption spectrum of the photochromic materials of the present disclosure may be observed. For example, FIG. 4a shows the absorption spectrum associated with the closed form of the photochromic material of Example 1 (i.e., 3,3-diphenyl-13-hydroxy-13-trifluoromethyl-3H,13H-indeno[2′,3′:3,4]naphtho[1,2-b]pyran), which is hypsochromically shifted compared to the closed form absorption spectrum of Comparative Example CE6 (i.e., 3,3-diphenyl-13-butyl-13-hydroxy-3H,13H-indeno[2′,3′:3,4]naphtho[1,2-b]pyran), shown in FIG. 5a. FIG. 4b shows the absorption spectrum associated with the closed form of the photochromic material of Example 7 (i.e., 3,3-di(4-methoxyphenyl)-6,11-dimethyl-13-trimethylsilyloxy-13-trifluoromethyl-3H,13H-indeno[2′,3′:3,4]naphtho[1,2-b]pyran), which is hypsochromically shifted compared to the closed form absorption spectrum of Comparative Example CE3 (i.e., 3,3-di(4-methoxyphenyl)-13-hydroxy-6,11,13-trimethyl-3H,13H-indeno[2′,3′:3,4]naphtho[1,2-b]pyran), shown in FIG. 5b. In addition, FIG. 4c shows the absorption spectrum associated with the closed form of the photochromic material of Example 11 (i.e., 3-(4-fluorophenyl)-3-(4-methoxyphenyl)-6-methoxy-7-morpholino-13-ethyl-13-(2,2,2-trifluoroethoxy)-3H,13H-indeno[2′,3′:3,4]naphtho[1,2-b]pyran), which is hypsochromically shifted compared to the closed form absorption spectrum of Comparative Example CE5 (i.e., 3-(4-fluorophenyl)-3-(4-methoxyphenyl)-6-methoxy-7-morpholino-13-ethyl-13-methoxy-3H,13H-indeno[2′,3′:3,4]naphtho[1,2-b]pyran), shown in FIG. 5c.

As previously discussed, while the present invention is described herein connection with certain embodiments and examples, the present invention is not limited to the particular embodiments and examples disclosed, but is intended to cover modifications that are within the spirit and scope of the invention, as defined by the appended claims. Further, it is to be understood that the present description illustrates aspects of the invention relevant to a clear understanding of the invention. Accordingly, certain aspects of the invention that would be apparent to those of ordinary skill in the art and that, therefore, would not facilitate a better understanding of the invention have not been presented in order to simplify the present description.

What is claimed is:

1. A photochromic material comprising:
(a) an indeno[2′,3′:3,4]naphtho[1,2-b]pyran; and
(b) a haloalkyl group bonded at the 13-position of the indeno[2′,3′:3,4]naphtho[1,2-b]pyran, wherein the haloalkyl group is:
(i) a perhalogenated group that is at least one of a perhalo(C₁-C₁₀)alkyl, a perhalo(C₂-C₁₀)alkenyl, a perhalo(C₃-C₁₀)alkynyl, a perhalo(C₁-C₁₀)alkoxy or a perhalo(C₃-C₁₀)cycloalkyl; or
(ii) a group represented by —O(CH₂)ₐ(CX₂)_{b}CT₃, wherein T is a halogen, each X is independently hydrogen or halogen, a is an integer ranging from 1 to 10, and b is an integer ranging from 1 to 10.

2. The photochromic material of claim 1 wherein the photochromic material has a ground-state form absorption spectrum for electromagnetic radiation having no absorption maxima in the visible region of the electromagnetic spectrum at wavelengths greater than 410 nm.

3. The photochromic material of claim 1 wherein the photochromic material has a closed-form absorption spectrum for electromagnetic radiation that is hypsochromically shifted as compared to a closed-form absorption spectrum for electromagnetic radiation of a photochromic material comprising a comparable indeno[2′,3′:3,4]naphtho[1,2-b]pyran without the haloalkyl group at the 13-position thereof.

4. The photochromic material of claim 1 wherein the haloalkyl group is a perhalo(C₁-C₁₀)alkyl represented by C_{d}F_{(2d+1)}, wherein d is an integer ranging from 1 to 10.

5. The photochromic material of claim 1 wherein the haloalkyl group is a group represented by —O(CH₂)ₐ(CX₂)_{b}CT₃, wherein T is fluorine, X is hydrogen or fluorine, a is an integer ranging from 1 to 10, and b is an integer ranging from 1 to 10.

6. The photochromic material of claim 1 wherein the 13-position of the indeno[2′,3′:3,4]naphtho[1,2-b]pyran is di-substituted with a first substituent and a second substituent, and wherein the first substituent is the haloalkyl group and the second substituent is one of:
(a) a perhalogenated group that is at least one of a perhalo(C₁-C₁₀)alkyl, a perhalo(C₂-C₁₀)alkenyl, a perhalo(C₃-C₁₀)alkynyl, a perhalo(C₁-C₁₀)alkoxy or a perhalo(C₃-C₁₀)cycloalkyl;
(b) a group represented by —O(CH₂)ₐ(CX₂)_{b}CT₃, wherein T is a halogen, each X is independently hydrogen or halogen, a is an integer ranging from 1 to 10, and b is an integer ranging from 1 to 10;
(c) a silicon-containing group represented by one of 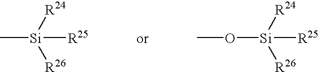
wherein R²⁴, R²⁵, and R²⁶ are each independently C₁-C₁₀ alkyl, C₁-C₁₀ alkoxy or phenyl;
(d) a metallocenyl group;
(e) a reactive substituent or a compatiblizing substituent;
(f) hydrogen, hydroxy, C₁-C₆ alkyl, chloro, fluoro, C₃-C₇ cycloalkyl, allyl or C₁-C₈ haloalkyl;
(g) morpholino, piperidino, pyrrolidino or an unsubstituted, mono- or di-substituted amino, wherein said amino substituents are each independently C₁-C₆ alkyl, phenyl, benzyl or naphthyl;
(h) an unsubstituted, mono-, di- or tri-substituted aryl group chosen from phenyl, naphthyl, benzyl, phenanthryl, pyrenyl, quinoyl, isoquinolyl, benzofuranyl, thienyl, benzothienyl, dibenzofuranyl, dibenzothienyl, carbazolyl or indolyl, wherein the aryl group substituents are each independently halogen, C₁-C₆ alkyl or C₁-C₆ alkoxy;
(i) —C(═O)R²⁷, wherein R²⁷ is hydrogen, hydroxy, C₁-C₆ alkyl, C₁-C₆ alkoxy, amino, mono- or di-(C₁-C₆)alkylamino, morpholino, piperidino, pyrrolidino, an unsubstituted, mono- or di-substituted phenyl or naphthyl, an unsubstituted, mono- or di-substituted phenoxy or an unsubstituted, mono- or di-substituted phenylamino, wherein said phenyl, naphthyl, phenoxy, and phenylamino substituents are each independently C₁-C₆ alkyl or C₁-C₆ alkoxy;
(j) —OR²⁸, wherein R²⁸ is
(i) C₁-C₆ alkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl, C₁-C₆ alkoxy(C₂-C₄)alkyl, C₃-C₇ cycloalkyl, mono(C₁-C₄)alkyl substituted C₃-C₇ cycloalkyl, C₁-C₈ chloroalkyl, C₁-C₈ fluoroalkyl, allyl or C₁-C₆ acyl;
(ii) —CH(R²⁹)R³⁰, wherein R²⁹ is hydrogen or C₁-C₃ alkyl, R³⁰ is —CN, —CF₃ or —COOR³¹, wherein R³¹ is hydrogen or C₁-C₃ alkyl; or
(iii) —C(═O)R³², wherein R³² is hydrogen, C₁-C₆ alkyl, C₁-C₆ alkoxy, amino, mono- or di-(C₁-C₆)alkylamino, an unsubstituted, mono- or di-substituted phenyl or naphthyl, an unsubstituted, mono- or di-substituted phenoxy or an unsubstituted, mono- or di-substituted phenylamino, wherein said phenyl, naphthyl, phenoxy and phenylamino substituents are each independently C₁-C₆ alkyl or C₁-C₆ alkoxy;
(k) a 4-substituted phenyl, the substituent being a dicarboxylic acid residue or derivative thereof, a diamine residue or derivative thereof, an amino alcohol residue or derivative thereof, a polyol residue or derivative thereof, —(CH₂)—, —(CH₂)ₑ— or —[O—(CH₂)ₑ]_{f}—, wherein e is an integer ranging from 2 to 6 and f is an integer ranging from 1 to 50, and wherein the substituent is connected to an aryl group of another photochromic material;
(l) —CH(R³³)₂, wherein R³³ is —CN or —COOR³⁴, wherein R³⁴ is hydrogen, C₁-C₆ alkyl, C₃-C₇ cycloalkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl or an unsubstituted, mono- or di-substituted aryl groups phenyl or naphthyl, wherein said phenyl and naphthyl substituents are each independently C₁-C₆ alkyl or C₁-C₆ alkoxy; or
(m) —CH(R³⁵)R³⁶, wherein R³⁵ is hydrogen, C₁-C₆ alkyl or an unsubstituted, mono- or di-substituted phenyl or naphthyl, wherein said phenyl or naphthyl substituents are each independently C₁-C₆ alkyl or C₁-C₆ alkoxy, and R³⁶ is —C(═O)OR³⁷, —C(═O)R³⁸ or —CH₂OR³⁹ wherein:
(i) R³⁷ is hydrogen, C₁-C₆ alkyl, C₃-C₇ cycloalkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl or an unsubstituted, mono- or di-substituted aryl groups phenyl or naphthyl, wherein said phenyl and naphthyl substituents are each independently C₁-C₆ alkyl or C₁-C₆ alkoxy;
(ii) R³⁸ is hydrogen, C₁-C₆ alkyl, amino, mono(C₁-C₆)alkylamino, di(C₁-C₆) alkylamino, phenylamino, diphenylamino, (mono- or di-(C₁-C₆)alkyl substituted phenyl)amino, (mono- or di-(C₁-C₆)alkoxy substituted phenyl)amino, (mono- or di-(C₁-C₆)alkyl substituted diphenyl)amino, (mono- or di-(C₁-C₆)alkoxy substituted diphenyl)amino, morpholino, piperidino or an unsubstituted, mono- or di-substituted phenyl or naphthyl, wherein said phenyl or naphthyl substituents are each independently C₁-C₆ alkyl or C₁-C₆ alkoxy; and
(iii) R³⁹ is hydrogen, —C(═O)R³⁷, C₁-C₆ alkyl, C₁-C₃ alkoxy (C₁-C₆)alkyl, phenyl(C₁-C₆)alkyl, mono-alkoxy substituted phenyl(C₁-C₆)alkyl or an unsubstituted, mono- or di-substituted phenyl or naphthyl, wherein said phenyl or naphthyl substituents are each independently C₁-C₆ alkyl or C₁-C₆ alkoxy.

7. The photochromic material of claim 6 wherein the first substituent is a perhalo(C₁-C₁₀) alkyl represented by —C_{d}F_{(2d+1)}, wherein d is an integer ranging from 1 to 10, and the second substituent is a silicon-containing group represented by 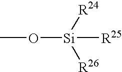
wherein are R²⁴, R²⁵ and R²⁶ are each independently C₁-C₁₀ alkyl or phenyl.

8. The photochromic material of claim 7 wherein d is 1, and at least two of R²⁴, R²⁵ and R²⁶ are methyl or phenyl.

9. A photochromic composition comprising the photochromic material of claim 1 incorporated into at least a portion of an organic material, said organic material being a polymeric material, an oligomeric material, a monomeric material or a mixture or combination thereof.

10. A photochromic article comprising:
a substrate; and
a photochromic material according to claim 1 connected to at least a portion of the substrate.

11. The photochromic article of claim 10 wherein the photochromic article is an optical element, said optical element being at least one of an ophthalmic element, a display element, a window, a mirror, and a liquid crystal cell element.

12. The photochromic article of claim 11 wherein the optical element is an ophthalmic element, said ophthalmic element being at least one of a corrective lens, a non-corrective lens, a magnifying lens, a protective lens, a visor, goggles and a lens for an optical instrument.

13. The photochromic article of claim 10 wherein the substrate comprises a polymeric material and the photochromic material is incorporated into at least a portion of the polymeric material by at least one of blending, bonding, and imbibing.

14. The photochromic article of claim 10 wherein the photochromic article comprises an at least partial coating connected to at least a portion of the substrate, said at least partial coating comprising the photochromic material.

15. The photochromic article of claim 14 wherein the photochromic article further comprises a protective coating on at least a portion of the at least partial coating comprising the photochromic material.

16. A photochromic indeno[2′,3′:3,4]naphtho[1,2-b]pyran comprising a haloalkyl group bonded at the 13-position of the indeno[2′,3′:3,4]naphtho[1,2-b]pyran, wherein the haloalkyl group is:
(a) a perhalogenated group that is at least one of a perhalo(C₁-C₁₀)alkyl, a perhalo(C₂-C₁₀)alkenyl, a perhalo(C₃-C₁₀)alkynyl, a perhalo(C₁-C₁₀)alkoxy or a perhalo(C₃-C₁₀)cycloalkyl; or
(b) a group represented by —O(CH₂)ₐ(CX₂)_{b}CT₃, wherein T is a halogen, each X is independently hydrogen or halogen, a is an integer ranging from 1 to 10, and b is an integer ranging from 1 to 10.

17. A photochromic material represented by: 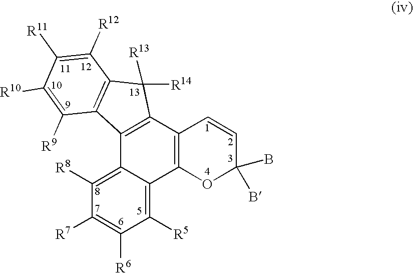
wherein:
R¹³ and R¹⁴ are each independently:
(a) a perhalogenated group that is at least one of a perhalo(C₁-C₁₀)alkyl, a perhalo(C₂-C₁₀)alkenyl, a perhalo(C₃-C₁₀)alkynyl, a perhalo(C₁-C₁₀)alkoxy or a perhalo(C₃-C₁₀)cycloalkyl;
(b) a group represented by —O(CH₂)ₐ(CX₂)_{b}CT₃, wherein T is a halogen, each X is independently hydrogen or halogen, a is an integer ranging from 1 to 10, and b is an integer ranging from 1 to 10;
(c) a silicon-containing group represented by one of 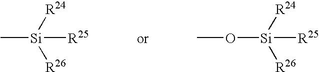
wherein R²⁴, R²⁵, and R²⁶ are each independently C₁-C₁₀ alkyl, C₁-C₁₀ alkoxy or phenyl;
(d) a metallocenyl group;
(e) a reactive substituent or a compatiblizing substituent;
(f) hydrogen, hydroxy, C₁-C₆ alkyl, chloro, fluoro, C₃-C₇ cycloalkyl, allyl or C₁-C₈ haloalkyl;
(g) morpholino, piperidino, pyrrolidino, an unsubstituted, mono- or di-substituted amino, wherein said amino substituents are each independently C₁-C₆ alkyl, phenyl, benzyl or naphthyl;
(h) an unsubstituted, mono-, di- or tri-substituted aryl group chosen from phenyl, naphthyl, benzyl, phenanthryl, pyrenyl, quinoyl, isoquinolyl, benzofuranyl, thienyl, benzothienyl, dibenzofuranyl, dibenzothienyl, carbazolyl or indolyl, wherein the aryl group substituents are each independently halogen, C₁-C₆ alkyl or C₁-C₆ alkoxy;
(i) —C(═O)R²⁷, wherein R²⁷ is hydrogen, hydroxy, C₁-C₆ alkyl, C₁-C₆ alkoxy, amino, mono- or di-(C₁-C₆)alkylamino, morpholino, piperidino, pyrrolidino, an unsubstituted, mono- or di-substituted phenyl or naphthyl, an unsubstituted, mono- or di-substituted phenoxy or an unsubstituted, mono- or di-substituted phenylamino, wherein said phenyl, naphthyl, phenoxy and phenylamino substituents are each independently C₁-C₆ alkyl or C₁-C₆ alkoxy;
(j) —OR²⁸, wherein R²⁸ is
(i) C₁-C₆ alkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl, C₁-C₆ alkoxy(C₂-C₄)alkyl, C₃-C₇ cycloalkyl, mono(C₁-C₄)alkyl substituted C₃-C₇ cycloalkyl, C₁-C₈ chloroalkyl, C₁-C₈ fluoroalkyl, allyl or C₁-C₆ acyl,
(ii) —CH(R²⁹)R³⁰, wherein R²⁹ is hydrogen or C₁-C₃ alkyl, R³⁰ is —CN, —CF₃ or —COOR³ , wherein R³¹ is hydrogen or C₁-C₃ alkyl, or
(iii) —C(═O)R³², wherein R³² is hydrogen, C₁-C₆ alkyl, C₁-C₆ alkoxy, amino, mono- or di-(C₁-C₆)alkylamino, an unsubstituted, mono- or di-substituted phenyl or naphthyl, an unsubstituted, mono- or di-substituted phenoxy or an unsubstituted, mono- or di-substituted phenylamino, wherein said phenyl, naphthyl, phenoxy and phenylamino substituents are each independently C₁-C₆ alkyl or C₁-C₆ alkoxy;
(k) a 4-substituted phenyl, the substituent being a dicarboxylic acid residue or derivative thereof, a diamine residue or derivative thereof, an amino alcohol residue or derivative thereof, a polyol residue or derivative thereof, —(CH₂)—, —(CH₂)ₑ— or —[O—(CH₂)ₑ]_{f}—, wherein e is an integer ranging from 2 to 6 and f is an integer ranging from 1 to 50, and wherein the substituent is connected to an aryl group of another photochromic material;
(l) —CH(R³³)₂, wherein R³³ is —CN or —COOR³⁴, wherein R³⁴ is hydrogen, C₁-C₆ alkyl, C₃-C₇ cycloalkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl or an unsubstituted, mono- or di-substituted phenyl or naphthyl, wherein said phenyl and naphthyl substituents are each independently C₁-C₆ alkyl or C₁-C₆ alkoxy; or
(m) —CH(R³⁵)R³⁶, wherein R³⁵ is hydrogen, C₁-C₆ alkyl or an unsubstituted, mono- or di-substituted phenyl or naphthyl, wherein said phenyl and naphthyl substituents are each independently C₁-C₆ alkyl or C₁-C₆ alkoxy, and R³⁶ is —C(═O)OR³⁷, —C(═O)R³⁸ or —CH₂OR³⁹ wherein:
(i) R³⁷ is hydrogen, C₁-C₆ alkyl, C₃-C₇ cycloalkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl or an unsubstituted, mono- or di-substituted phenyl or naphthyl, wherein said phenyl and naphthyl substituents are each independently C₁-C₆ alkyl or C₁-C₆ alkoxy,
(ii) R³⁸ is hydrogen, C₁-C₆ alkyl, amino, mono(C₁-C₆)alkylamino, di(C₁-C₆) alkylamino, phenylamino, diphenylamino, (mono- or di-(C₁-C₆)alkyl substituted phenyl)amino, (mono- or di-(C₁-C₆)alkoxy substituted phenyl)amino, di(mono- or di-(C₁-C₆)alkyl substituted phenyl)amino, di(mono- or di-(C₁-C₆)alkoxy substituted phenyl)amino, morpholino, piperidino or an unsubstituted, mono- or di-substituted phenyl or naphthyl, wherein said phenyl or naphthyl substituents are each independently C₁-C₆ alkyl or C₁-C₆ alkoxy, and
(iii) R³⁹ is hydrogen, —C(═O)R³⁷, C₁-C₆ alkyl, C₁-C₃ alkoxy (C₁-C₆)alkyl, phenyl(C₁-C₆)alkyl, mono-alkoxy substituted phenyl(C₁-C₆)alkyl or an unsubstituted, mono- or di-substituted phenyl or naphthyl, wherein said phenyl or naphthyl substituents are each independently C₁-C₆ alkyl or C₁-C₆ alkoxy;
provided that at least one of R¹³ and R¹⁴ is a perhalogenated group that is at least one of a perhalo(C₁-C₁₀)alkyl, a perhalo(C₂-C₁₀)alkenyl, a perhalo(C₃-C₁₀)alkynyl, a perhalo(C₁-C₁₀)alkoxy or a perhalo(C₃-C₁₀)cycloalkyl, or a group represented by —O(CH₂)ₐ(CX₂)_{b}CT₃;
B and B′ are each independently:
(a) a metallocenyl group;
(b) an aryl group that is mono-substituted with a reactive substituent or a compatiblizing substituent;
(c) 9-julolidinyl, an unsubstituted, mono-, di- or tri-substituted aryl group chosen from phenyl and naphthyl, an unsubstituted, mono- or di-substituted heteroaromatic group chosen from pyridyl, furanyl, benzofuran-2-yl, benzofuran-3-yl, thienyl, benzothien-2-yl, benzothien-3-yl, dibenzofuranyl, dibenzothienyl, carbazoyl, benzopyridyl, indolinyl or fluorenyl, wherein the aryl and heteroaromatic substituents are each independently:
hydroxy, aryl, mono- or di-(C₁-C₁₂)alkoxyaryl, mono- or di-(C₁-C₁₂)alkylaryl, haloaryl, C₃-C₇ cycloalkylaryl, C₃-C₇ cycloalkyl, C₃-C₇ cycloalkyloxy, C₃-C₇ cycloalkyloxy(C₁-C₁₂)alkyl, C₃-C₇ cycloalkyloxy(C₁-C₁₂)alkoxy, aryl(C₁-C₁₂)alkyl, aryl(C₁-C₁₂)alkoxy, aryloxy, aryloxy(C₁-C₁₂)alkyl, aryloxy(C₁-C₁₂)alkoxy, mono- or di-(C₁-C₁₂)alkylaryl(C₁-C₁₂)alkyl, mono- or di-(C₁-C₁₂)alkoxyaryl(C₁-C₁₂)alkyl, mono- or di-(C₁-C₁₂)alkylaryl(C₁-C₁₂)alkoxy, mono- or di-(C₁-C₁₂)alkoxyaryl(C₁-C₁₂)alkoxy, amino, mono- or di-(C₁-C₁₂)alkylamino, diarylamino, piperazino, N—(C₁-C₁₂)alkylpiperazino, N-arylpiperazino, aziridino, indolino, piperidino, morpholino, thiomorpholino, tetrahydroquinolino, tetrahydroisoquinolino, pyrrolidino, C₁-C₁₂ alkyl, C₁-C₁₂ haloalkyl, C₁-C₁₂ alkoxy, mono(C₁-C₁₂)alkoxy(C₁-C₁₂)alkyl, acryloxy, methacryloxy, halogen or —C(═O)R¹⁵, wherein R¹⁵ is —OR¹⁶, —N(R¹⁷)R¹⁸, piperidino or morpholino, wherein R¹⁶ is allyl, C₁-C₆ alkyl, phenyl, mono(C₁-C₆)alkyl substituted phenyl, mono(C₁-C₆)alkoxy substituted phenyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl, C₁-C₆ alkoxy(C₂-C₄)alkyl or C₁-C₆ haloalkyl, and R¹⁷ and R¹⁸ are each independently C₁-C₆ alkyl, C₅-C₇ cycloalkyl or a substituted or an unsubstituted phenyl, wherein said phenyl substituents are each independently C₁-C₆ alkyl or C₁-C₆ alkoxy;
(d) an unsubstituted or mono-substituted group chosen from pyrazolyl, imidazolyl, pyrazolinyl, imidazolinyl, pyrrolidino, phenothiazinyl, phenoxazinyl, phenazinyl and acridinyl, said wherein said substituents are each independently C₁-C₁₂ alkyl, C₁-C₁₂ alkoxy, phenyl or halogen;
(e) a 4-substituted phenyl, the substituent being a dicarboxylic acid residue or derivative thereof, a diamine residue or derivative thereof, an amino alcohol residue or derivative thereof, a polyol residue or derivative thereof, —(CH₂)—, —(CH₂)ₑ— or —[O—(CH₂)ₑ]_{f}—, wherein e is an integer ranging from 2 to 6 and f is an integer ranging from 1 to 50, and wherein the substituent is connected to an aryl group of another photochromic material;
(f) a group represented by: 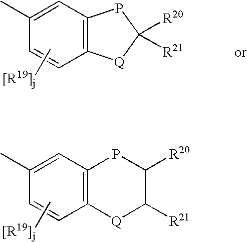
wherein P is —CH₂— or —O—; Q is —O— or substituted nitrogen, the substituted nitrogen substituents being hydrogen, C₁-C₁₂ alkyl or C₁-C₁₂ acyl, provided that when Q is substituted nitrogen, P is —CH₂—; each R¹⁹ is independently C₁-C₁₂ alkyl, C₁-C₁₂ alkoxy, hydroxy or halogen; R²⁰ and R²¹ are each independently hydrogen or C₁-C₁₂ alkyl; and j is an integer ranging from 0 to 2; or
(g) a group represented by: 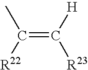
wherein R²² is hydrogen or C₁-C₁₂ alkyl; and R²³ is an unsubstituted, mono- or di-substituted naphthyl, phenyl, furanyl or thienyl, wherein said naphthyl, phenyl, furanyl and thienyl substituents are C₁-C₁₂ alkyl, C₁-C₁₂ alkoxy or halogen; or
B and B′ taken together form a fluoren-9-ylidene or mono- or di-substituted fluoren-9-ylidene, wherein said fluoren-9-ylidene substituents are each independently C₁-C₁₂ alkyl, C₁-C₁₂ alkoxy or halogen;
R⁵, R⁸, R⁹ and R¹² are each independently:
(a) hydrogen, C₁-C₆ alkyl, chloro, fluoro, bromo, C₃-C₇ cycloalkyl or a unsubstituted, mono- or di-substituted phenyl, wherein said phenyl substituents are each independently C₁-C₆ alkyl or C₁-C₆ alkoxy;
(b) —OR⁴⁰ or —OC(═O)R⁴⁰, wherein R⁴⁰ is hydrogen, amine, alkylene glycol, polyalkylene glycol, C₁-C₆ alkyl, phenyl(C₁-C₃)alkyl, mono(C₁-C₆)alkyl substituted phenyl(C₁-C₃)alkyl, mono(C₁-C₆)alkoxy substituted phenyl(C₁-C₃)alkyl, (C₁-C₆)alkoxy(C₂-C₄)alkyl, C₃-C₇ cycloalkyl, mono(C₁-C₄)alkyl substituted C₃-C₇ cycloalkyl or an unsubstituted, mono- or di-substituted phenyl, wherein said phenyl substituents are each independently C₁-C₆ alkyl or C₁-C₆ alkoxy;
(c) a reactive substituent or a compatiblizing substituent;
(d) a 4-substituted phenyl, the substituent being a dicarboxylic acid residue or derivative thereof, a diamine residue or derivative thereof, an amino alcohol residue or derivative thereof, a polyol residue or derivative thereof, —(CH₂)—, —(CH₂)ₑ— or —[O—(CH₂)ₑ]_{f}—, wherein e is an integer ranging from 2 to 6, and f is an integer ranging from 1 to 50, and wherein the substituent is connected to an aryl group of another photochromic material;
(e) —N(R⁴¹)R⁴², wherein R⁴¹ and R⁴² are each independently hydrogen, C₁-C₈ alkyl, phenyl, naphthyl, furanyl, benzofuran-2-yl, benzofuran-3-yl, thienyl, benzothien-2-yl, benzothien-3-yl, dibenzofuranyl, dibenzothienyl, benzopyridyl, fluorenyl, C₁-C₈ alkylaryl, C₃-C₈ cycloalkyl, C₄-C₁₆ bicycloalkyl, C₅-C₂₀ tricycloalkyl or C₁-C₂₀ alkoxy(C₁-C₆)alkyl, or R⁴¹ and R⁴² come together with the nitrogen atom to form a C₃-C₂₀ hetero-bicycloalkyl ring or a C₄-C₂₀ hetero-tricycloalkyl ring;
(f) a nitrogen containing ring represented by: 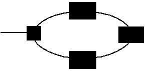
wherein each —V— is independently chosen for each occurrence from —CH₂—, —CH(R⁴³)—, —C(R⁴³)₂—, —CH(aryl)-, —C(aryl)₂— and —C(R⁴³)(aryl)-, wherein each R⁴³ is independently C₁-C₆ alkyl and each aryl is independently phenyl or naphthyl; —W— is —V—, —O—, —S—, —S(O)—, —SO₂—, —NH—, —N(R⁴³)— or —N(aryl)-; s is an integer ranging from 1 to 3; and r is an integer ranging from 0 to 3, provided that if r is 0, then —W— is the same as —V—;
(g) a group represented by: 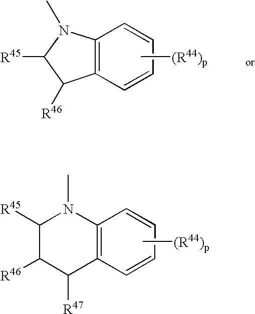
wherein each R⁴⁴ is independently C₁-C₆ alkyl, C₁-C₆ alkoxy, fluoro or chloro; R⁴⁵, R⁴⁶ and R⁴⁷ are each independently hydrogen, C₁-C₆ alkyl, phenyl or naphthyl, or R⁴⁵ and R⁴⁶ together form a ring of 5 to 8 carbon atoms, and p is an integer ranging from 0 to 3; or
(h) a substituted or unsubstituted C₄-C₁₈ spirobicyclic amine or a substituted or unsubstituted C₄-C₁₈ spirotricyclic amine, wherein said substituents are each independently aryl, C₁-C₆ alkyl, C₁-C₆ alkoxy or phenyl(C₁-C₆)alkyl;
R⁷ and R¹⁰ are each independently:
(a) any of the groups discussed above with respect to R⁵, R⁸, R⁹ and R¹²; or
(b) a metallocenyl group;
R⁶ and R¹¹ are each independently:
(c) any of the groups discussed above with respect to R⁷ and R¹⁰;
(d) perfluoroalkyl or perfluoroalkoxy;
(e) —C(═O)R⁴⁸ or —SO₂R⁴⁸, wherein R⁴⁸ is hydrogen, C₁-C₆ alkyl, —OR⁴⁹ or —NR⁵⁰R⁵¹, wherein R⁴⁹, R⁵⁰ and R⁵¹ are each independently hydrogen, C₁-C₆ alkyl, C₅-C₇ cycloalkyl, alkylene glycol, polyalkylene glycol or an unsubstituted, mono- or di-substituted phenyl, wherein said phenyl substituents are each independently C₁-C₆ alkyl or C₁-C₆ alkoxy;
(f) —C(═C(R⁵²)₂)R⁵³, wherein each R⁵² is independently —C(═O)R⁴⁸, —OR⁴⁹, —OC(═O)R⁴⁹, —NR⁵⁰R⁵¹, hydrogen, halogen, cyano, C₁-C₆ alkyl, C₅-C₇ cycloalkyl, alkylene glycol, polyalkylene glycol or an unsubstituted, mono- or di-substituted phenyl, wherein said phenyl substituents are each independently C₁-C₆ alkyl or C₁-C₆ alkoxy; and R⁵³ is hydrogen, C₁-C₆ alkyl, C₅-C₇ cycloalkyl, alkylene glycol, polyalkylene glycol or an unsubstituted, mono- or di-substituted phenyl, wherein said phenyl substituents are each independently C₁-C₆ alkyl or C₁-C₆ alkoxy; or
(g) —C≡CR⁵⁴ or —C≡N, wherein R⁵⁴ is —C(═O)R⁴⁸, hydrogen, C₁-C₆ alkyl, C₅-C₇ cycloalkyl or an unsubstituted, mono- or di-substituted phenyl, wherein said phenyl substituents are each independently C₁-C₆ alkyl or C₁-C₆ alkoxy; or
at least one pair of adjacent groups R⁶ and R⁷ or R¹⁰ and R¹¹ together form a group represented by: 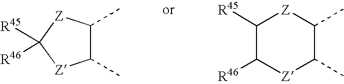
wherein Z and Z′ are each independently oxygen or the group —NR⁴¹—; or
R⁶ and R⁷ or R¹⁰ and R¹¹ together form an aromatic or heteroaromatic fused group, said fused group being benzo, indeno, dihydronaphthalene, indole, benzofuran, benzopyran or thianaphthene.

18. The photochromic material of claim 17, wherein at least one of R¹³ and R¹⁴ a perhalogenated C₁-C₁₀ alkyl represented by —C_{d}F₍₂₊₁₎, wherein d is an integer ranging from 1 to 10, and at least one of R¹³ and R¹⁴ is a silicon-containing group represented by 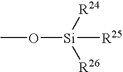
wherein R²⁴, R²⁵ and R²⁶ are each independently C₁-C₁₀ alkyl or phenyl.

19. The photochromic material of claim 17, wherein the photochromic material is
a) 3,3-diphenyl-13-hydroxy-13-trifluoromethyl-3H,13H-indeno[2′,3′:3,4]naphtho[1,2-b]pyran;
b) 3,3-diphenyl-13-trimethylsiloxy-13-trifluoromethyl-3H,13H-indeno[2′,3′:3,4]naphtho[1,2-b]pyran;
c) 3,3-di(4-methoxyphenyl)-6,11-dimethyl-13-trimethylsiloxy-13-trifluoromethyl-3H,13H-indeno[2′,3′:3,4]naphtho[1,2-b]pyran;
d) 3,3-di(4-methoxyphenyl)-6,11,13-trimethyl-13-(2,2,2-trifluoroethoxy)-3H,13H-indeno[2′,3′:3,4]naphtho[1,2-b]pyran;
e) 3,3-di(4-methoxyphenyl)-6-methoxy-7-morpholino-13-ethyl-13-(2,2,2-trifluoroethoxy)-3H,13H-indeno[2′,3′:3,4]naphtho[1,2-b]pyran;
f) 3-(4-fluorophenyl)-3-(4-methoxyphenyl)-6-methoxy-7-morpholino-13-butyl-13-(2,2,2-trifluoroethoxy)-3H,13H-indeno[2′,3′:3,4]naphtho[1,2-b]pyran;
g) 3-(4-fluorophenyl)-3-(4-methoxyphenyl)-6-methoxy-7-morpholino-13-ethyl-13-(2,2,2-trifluoroethoxy)-3H,13H-indeno[2′,3′:3,4]naphtho[1,2-b]pyran;
h) 3-(4-fluorophenyl)-3-(4-methoxyphenyl)-6-methoxy-7-morpholino-13-butyl-13-(1H,1H,2H,2H-perfluorododecanoxy)-3H,13H-indeno[2′,3′:3,4]naphtho[1,2-b]pyran;
i) 3-(4-fluorophenyl)-3-(4-methoxyphenyl)-6-methoxy-7-morpholino-13-butyl-13-(3-perfluorobutylpropoxy)-3II,13II-indeno[2′,3′:3,4]naphtho[1,2-b]pyran;
j) 3,3-di(4-methoxyphenyl)-6-methoxy-7-morpholino-13-ethyl-13-(2,2,3,3,3-pentafluoropropoxy)-3H,13H-indeno[2′,3′:3,4]naphtho[1,2-b]pyran;
k) 3,3-di(4-methoxyphenyl)-6-methoxy-7-morpholino-13-ethyl-13-(2,2,3,3,4,4,4-heptafluorobutoxy)-3H, 13H-indeno[2′,3′:3,4]naphtho[1,2-b]pyran; or
l) mixtures thereof.
